SEQUENCE LISTING (24 sequences)

SEQ ID NO: 1 - DNA, 49 bp, synthetic construct
  source 1..49: mol_type = other DNA
ggctgcaggtccgatccaccggtcgccaccatggctctcccagtgactg

SEQ ID NO: 2 - DNA, 48 bp, synthetic construct
  source 1..48: mol_type = other DNA
tcgagtcgacgactccggaacgaattctgattagcgagggggcagggc

SEQ ID NO: 3 - DNA, 49 bp, synthetic construct; identical to SEQ ID NO: 1
  source 1..49: mol_type = other DNA

SEQ ID NO: 4 - DNA, 55 bp, synthetic construct
  source 1..55: mol_type = other DNA
tcgagtcgacgactccggaacgaattctgattacttgctccgcacccagaagatg

SEQ ID NO: 5 - DNA, 39 bp, synthetic construct
  source 1..39: mol_type = other DNA
aagcgcagcggctccggcgagggccggggcagcctgctg

SEQ ID NO: 6 - DNA, 45 bp, synthetic construct
  source 1..45: mol_type = other DNA
gcagtcactgggagagccatgggtccggggttctcttccacgtcg

SEQ ID NO: 7 - DNA, 26 bp, synthetic construct
  source 1..26: mol_type = other DNA
ggatctggagcaacaaacttctcact

SEQ ID NO: 8 - DNA, 48 bp, synthetic construct
  source 1..48: mol_type = other DNA
aagcagtagggcagtcactgggagagccataggcccgggattctcctc

SEQ ID NO: 9 - DNA, 45 bp, synthetic construct
  source 1..45: mol_type = other DNA
aagtttgttgctccagatccccatggcaggaggctgagcaggagg

SEQ ID NO: 10 - DNA, 45 bp, synthetic construct; identical to SEQ ID NO: 9
  source 1..45: mol_type = other DNA

SEQ ID NO: 11 - DNA, 48 bp, synthetic construct
  source 1..48: mol_type = other DNA
ccctaaaagctagcgccaccatggagcaacaggatcagagcatgaagg

SEQ ID NO: 12 - DNA, 51 bp, synthetic construct
  source 1..51: mol_type = other DNA
agtgagaagtttgttgctccagatccctgttccgaagtgacaggtggaagc

SEQ ID NO: 13 - DNA, 4971 bp, synthetic construct
  source 1..4971: mol_type = other DNA
atgccagggaggacctgggagctgtgcctgctactgctgctggggctgggactggggtcccaggaggccctacccccaccctgtgagagtgagatttactgccacggggagctcctaaaccaagttcaaatggccaagctctaccaggatgacaagcagtttgtggacatgccactgtctatagctccagaacaagtcctgcagaccttcactgagctgtccagggaccacaatcacagcatccccagggagcagctgcaggcgtttgtccacgaacacttccaggccaaggggcaggagctgcagccctggacccctgcagactggaaagacagcccccagttcctgcagaagatttcagatgccaaactgcgtgcctgggcagggcagctgcatcagctctggaagaagctggggaagaagatgaagccagaggttctcagccaccctgagcggttctctctcatctactcagaacatcccttcattgtgcctggcggtcgctttgttgagttctactactgggactcctactgggtcatggagggtctgctcctctcagagatggctgagacggtgaagggcatgctgcagaacttcttggacctggtgaaaacctatgggcatgtccccaatggtgggcgcgtgtactacctgcagcggagccagcccccactcttgaccctcatgatggattgctacttgactcacaccaatgacaccgcctttctacaggaaaacattgaaacactagccttggaattggacttttggaccaagaacaggactgtctctgtgagcttggagggaaagaactacctcctgaatcgctattatgtcccttatgggggacccaggcctgagtcctacagcaaagatgtggagttggctgacaccttgccagaaggagaccgggaggctctgtgggctgagctcaaggctggggctgagtctggctgggacttctcttcacgctggctcattggaggcccaaaccccaactcgcttagcggcatccgaacaagcaaactggtgcctgttgacctgaatgccttcctatgccaagcagaggagctgatgagcaacttctattccaggctggggaacgactcccaggccacgaagtacagaatcctgcggtcgcagcgcttggccgccctgaacacagtcctgtgggatgagcagaccggagcctggttcgattacgaccttgagaagaagaagaaaaaccgggagttttacccatccaacctcactccactctgggccgggtgtttctctgaccctggcgtggcggacaaggctctgaaatacctggaggacaaccggatcctgacttaccagtatgggatcccgacctctctccagaagacaggccagcagtgggatttccccaatgcctgggcccccctgcaggacctggtcatcagaggcctggccaaggcacctttacgtcgggcccaggaagtggctttccagctggctcagaattggatccgaaccaattttgatgtctactcgcagaagtcagccatgtatgagaagtatgacgtcagcaacggtggacagcccggtgggggaggagaatatgaagttcaggagggatttggctggacgaatggcgtggtcctgatgctgctggaccgctatggtgaccggctgacctcaggggccaagctggctttcctggagccccactgcctggcggccacccttctgcccagcctcctgctcagcctcctgccatggggatctggagcaacaaacttctcactactcaaacaagcaggtgacgtggaggagaatcccgggcctatgggctggctgtgttccggcctgctgtttcctgtgtcctgtctggtgctgctgcaggtggccagctccgggaacatgaaagtgctgcaggagcccacatgtgtgtccgactacatgtccatctctacatgtgagtggaagatgaacggccccacaaactgctctaccgagctgcggctgctgtaccagctggtgtttctgctgagcgaggcccacacctgtatcccagaaaataatggcggggccgggtgtgtgtgccacctgctgatggatgacgtggtgtctgccgacaattacaccctggacctgtgggccggacagcagctgctgtggaaggggtccttcaaaccctctgagcacgtgaagccaagggcccccggcaacctgacagtgcacaccaacgtgtctgatacactgctgctgacatggagcaatccataccctcctgacaactacctgtacaaccacctgacctacgccgtgaatatctggagcgaaaatgatcctgccgactttcggatttacaatgtgacctatctggagccctccctgagaattgccgcctctaccctgaaatctggaatctcctaccgcgccagggtgcgggcctgggcccagtgttacaacaccacctggtctgagtggagcccaagcaccaagtggcacaattcttatcgggagccttttgagcagcacctgatcccctggctgggacacctgctggtggggctgtctggcgcctttggcttcatcattctggtgtacctgctgatcaactgtaggaatacaggcccttggctgaagaaggtgctgaagtgtaacacccccgacccctctaagttcttcagccagctgtcctctgaacacgggggagatgtgcagaagtggctgtccagccctttcccatccagctcctttagccccgggggcctggcccctgagatctctccactggaagtgctggagcgggacaaggtgacccagctgctgctgcagcaggacaaggtgccagaacccgcctccctgagctccaaccacagcctgacatcttgctttacaaatcagggatacttcttcttccacctgcccgatgccctggagatcgaggcctgccaggtgtacttcacctacgatccctactctgaggaagacccagatgagggcgtggccggggccccaaccgggtccagcccacagccactgcagccactgtccggcgaagatgacgcctactgcacattcccttccagggatgacctgctgctgttcagcccatctctgctgggcggaccctctcctccaagcacagccccagggggatccggcgccggggaagagaggatgccccctagcctgcaggagcgcgtgcccagagactgggacccccagcccctgggccctccaacccctggggtgcccgacctggtggacttccagcctccacccgagctggtgctgagggaggccggcgaagaggtgcccgacgccggcccccgggagggcgtgtccttcccttggtccagacctccaggacagggcgagttccgcgccctgaacgccaggctgcctctgaacaccgatgcctacctgtctctgcaggaactgcagggccaggacccaacccacctggtgcggagaaagcgcagcggctccggcgagggccggggcagcctgctgacctgcggcgacgtggaagagaaccccggacccatggctctcccagtgactgccctactgcttcccctagcgcttctcctgcatgcagaggtgcagctgcagcagtcaggacctgaactggtgaagcctgggacttcagtgaggatatcctgcaagacttctggatacacattcactgaatataccatacactgggtgaagcagagccatggaaagagccttgagtggattggaaacatcaatcctaacaatggtggtaccacctacaatcagaagttcgaggacaaggccacattgactgtagacaagtcctccagtacagcctacatggagctccgcagcctaacatctgaggattctgcagtctattattgtgcagctggttggaactttgactactggggccaagggaccacggtcaccgtctcctcaggtggaggtggatcaggtggaggtggatctggtggaggtggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaatgttcagtctgaagacttggcagattatttctgtcagcaatataacagctatcccctcacgttcggtgctgggaccatgctggacctgaaacgggcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 1657 aa, synthetic construct
MPGRTWELCLLLLLGLGLGSQEALPPPCESEIYCHGELLNQVQMAKLYQDDKQFVDMPLSIAPEQVLQTFTELSRDHNHSIPREQLQAFVHEHFQAKGQELQPWTPADWKDSPQFLQKISDAKLRAWAGQLHQLWKKLGKKMKPEVLSHPERFSLIYSEHPFIVPGGRFVEFYYWDSYWVMEGLLLSEMAETVKGMLQNFLDLVKTYGHVPNGGRVYYLQRSQPPLLTLMMDCYLTHTNDTAFLQENIETLALELDFWTKNRTVSVSLEGKNYLLNRYYVPYGGPRPESYSKDVELADTLPEGDREALWAELKAGAESGWDFSSRWLIGGPNPNSLSGIRTSKLVPVDLNAFLCQAEELMSNFYSRLGNDSQATKYRILRSQRLAALNTVLWDEQTGAWFDYDLEKKKKNREFYPSNLTPLWAGCFSDPGVADKALKYLEDNRILTYQYGIPTSLQKTGQQWDFPNAWAPLQDLVIRGLAKAPLRRAQEVAFQLAQNWIRTNFDVYSQKSAMYEKYDVSNGGQPGGGGEYEVQEGFGWTNGVVLMLLDRYGDRLTSGAKLAFLEPHCLAATLLPSLLLSLLPWGSGATNFSLLKQAGDVEENPGPMGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVRRKRSGSGEGRGSLLTCGDVEENPGPMALPVTALLLPLALLLHAEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTVTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 12143 bp, synthetic construct
  source 1..12143: mol_type = other DNA
ttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagctagcgccaccatggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggttggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggctggccgatcctgctggggctgaccggggtccccgcggcgctgcagctccttctgctgcccttcttccccgagagccccaggtacctgctgattcagaagaaagacgaagcggccgccaagaaagccctacagacgctgcgcggctgggactctgtggacagggaggtggccgagatccggcaggaggatgaggcagagaaggccgcgggcttcatctccgtgctgaagctgttccggatgcgctcgctgcgctggcagctgctgtccatcatcgtcctcatgggcggccagcagctgtcgggcgtcaacgctatctactactacgcggaccagatctacctgagcgccggcgtgccggaggagcacgtgcagtacgtgacggccggcaccggggccgtgaacgtggtcatgaccttctgcgccgtgttcgtggtggagctcctgggtcggaggctgctgctgctgctgggcttctccatctgcctcatagcctgctgcgtgctcactgcagctctggcactgcaggacacagtgtcctggatgccatacatcagcatcgtctgtgtcatctcctacgtcataggacatgccctcgggcccagtcccatacccgcgctgctcatcactgagatcttcctgcagtcctctcggccatctgccttcatggtggggggcagtgtgcactggctctccaacttcaccgtgggcttgatcttcccgttcatccaggagggcctcggcccgtacagcttcattgtcttcgccgtgatctgcctcctcaccaccatctacatcttcttgattgtcccggagaccaaggccaagacgttcatagagatcaaccagattttcaccaagatgaataaggtgtctgaagtgtacccggaaaaggaggaactgaaagagcttccacctgtcacttcggaacagggatctggagcaacaaacttctcactactcaaacaagcaggtgacgtggaggagaatcccgggcctatgggctggctgtgttccggcctgctgtttcctgtgtcctgtctggtgctgctgcaggtggccagctccgggaacatgaaagtgctgcaggagcccacatgtgtgtccgactacatgtccatctctacatgtgagtggaagatgaacggccccacaaactgctctaccgagctgcggctgctgtaccagctggtgtttctgctgagcgaggcccacacctgtatcccagaaaataatggcggggccgggtgtgtgtgccacctgctgatggatgacgtggtgtctgccgacaattacaccctggacctgtgggccggacagcagctgctgtggaaggggtccttcaaaccctctgagcacgtgaagccaagggcccccggcaacctgacagtgcacaccaacgtgtctgatacactgctgctgacatggagcaatccataccctcctgacaactacctgtacaaccacctgacctacgccgtgaatatctggagcgaaaatgatcctgccgactttcggatttacaatgtgacctatctggagccctccctgagaattgccgcctctaccctgaaatctggaatctcctaccgcgccagggtgcgggcctgggcccagtgttacaacaccacctggtctgagtggagcccaagcaccaagtggcacaattcttatcgggagccttttgagcagcacctgatcccctggctgggacacctgctggtggggctgtctggcgcctttggcttcatcattctggtgtacctgctgatcaactgtaggaatacaggcccttggctgaagaaggtgctgaagtgtaacacccccgacccctctaagttcttcagccagctgtcctctgaacacgggggagatgtgcagaagtggctgtccagccctttcccatccagctcctttagccccgggggcctggcccctgagatctctccactggaagtgctggagcgggacaaggtgacccagctgctgctgcagcaggacaaggtgccagaacccgcctccctgagctccaaccacagcctgacatcttgctttacaaatcagggatacttcttcttccacctgcccgatgccctggagatcgaggcctgccaggtgtacttcacctacgatccctactctgaggaagacccagatgagggcgtggccggggccccaaccgggtccagcccacagccactgcagccactgtccggcgaagatgacgcctactgcacattcccttccagggatgacctgctgctgttcagcccatctctgctgggcggaccctctcctccaagcacagccccagggggatccggcgccggggaagagaggatgccccctagcctgcaggagcgcgtgcccagagactgggacccccagcccctgggccctccaacccctggggtgcccgacctggtggacttccagcctccacccgagctggtgctgagggaggccggcgaagaggtgcccgacgccggcccccgggagggcgtgtccttcccttggtccagacctccaggacagggcgagttccgcgccctgaacgccaggctgcctctgaacaccgatgcctacctgtctctgcaggaactgcagggccaggacccaacccacctggtgcggagaaagcgcagcggctccggcgagggccggggcagcctgctgacctgcggcgacgtggaagagaaccccggacccatggctctcccagtgactgccctactgcttcccctagcgcttctcctgcatgcagaggtgcagctgcagcagtcaggacctgaactggtgaagcctgggacttcagtgaggatatcctgcaagacttctggatacacattcactgaatataccatacactgggtgaagcagagccatggaaagagccttgagtggattggaaacatcaatcctaacaatggtggtaccacctacaatcagaagttcgaggacaaggccacattgactgtagacaagtcctccagtacagcctacatggagctccgcagcctaacatctgaggattctgcagtctattattgtgcagctggttggaactttgactactggggccaagggaccacggtcaccgtctcctcaggtggaggtggatcaggtggaggtggatctggtggaggtggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaatgttcagtctgaagacttggcagattatttctgtcagcaatataacagctatcccctcacgttcggtgctgggaccatgctggacctgaaacgggcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatcagaattcgttccggagtcgtcgactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcatcttcgccttcgccctcagacgagtcggatctccctttgggccgcctccccgcctggaattaattcgagctcggtacctttaagaccaatgacttacaaggcagctgtagatcttagccactttttaaaagaaaaggggggactggaagggctacgtaactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctcacttgagcgtcgatttttgtgatgctcgtcaggggggcggagcctatggaaaaacgccagcaacgcggcctttttacggttcctggccttttgctggccttttgctcacatg

SEQ ID NO: 16 - protein, 1575 aa, synthetic construct
MEQQDQSMKEGRLTLVLALATLIAAFGSSFQYGYNVAAVNSPALLMQQFYNETYYGRTGEFMEDFPLTLLWSVTVSMFPFGGFIGSLLVGPLVNKFGRKGALLFNNIFSIVPAILMGCSRVATSFELIIISRLLVGICAGVSSNVVPMYLGELAPKNLRGALGVVPQLFITVGILVAQIFGLRNLLANVDGWPILLGLTGVPAALQLLLLPFFPESPRYLLIQKKDEAAAKKALQTLRGWDSVDREVAEIRQEDEAEKAAGFISVLKLFRMRSLRWQLLSIIVLMGGQQLSGVNAIYYYADQIYLSAGVPEEHVQYVTAGTGAVNVVMTFCAVFVVELLGRRLLLLLGFSICLIACCVLTAALALQDTVSWMPYISIVCVISYVIGHALGPSPIPALLITEIFLQSSRPSAFMVGGSVHWLSNFTVGLIFPFIQEGLGPYSFIVFAVICLLTTIYIFLIVPETKAKTFIEINQIFTKMNKVSEVYPEKEELKELPPVTSEQGSGATNFSLLKQAGDVEENPGPMGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVRRKRSGSGEGRGSLLTCGDVEENPGPMALPVTALLLPLALLLHAEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTVTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 3321 bp, synthetic construct
  source 1..3321: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaggctgctgccggtgtatgagctcagtggagagcaccatgacgaagagtggtcagtgaagacttaccaggaagtagctcagaagtttgtggaaactcaccctgagtttattggaatcaaaatcatttattcggatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggcggaggctccggcggaggcggaagcggcggcggaggatctgactacaaggacgacgatgacaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcggatctggagcaacaaacttctcactactcaaacaagcaggtgacgtggaggagaatcccgggcctatggctctcccagtgactgccctactgcttcccctagcgcttctcctgcatgcagaggtgcagctgcagcagtcaggacctgaactggtgaagcctgggacttcagtgaggatatcctgcaagacttctggatacacattcactgaatataccatacactgggtgaagcagagccatggaaagagccttgagtggattggaaacatcaatcctaacaatggtggtaccacctacaatcagaagttcgaggacaaggccacattgactgtagacaagtcctccagtacagcctacatggagctccgcagcctaacatctgaggattctgcagtctattattgtgcagctggttggaactttgactactggggccaagggaccacggtcaccgtctcctcaggtggaggtggatcaggtggaggtggatctggtggaggtggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaatgttcagtctgaagacttggcagattatttctgtcagcaatataacagctatcccctcacgttcggtgctgggaccatgctggacctgaaacgggcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - protein, 1107 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGGGSGGGGSGGGGSDYKDDDDKTTTPAPRPPTPAPTIASQPLSLRPEACRPAAGGAVHTRGLDFACDIYIWAPLAGTCGVLLLSLVITLYCGSGATNFSLLKQAGDVEENPGPMALPVTALLLPLALLLHAEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTVTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 35 bp, synthetic construct
  source 1..35: mol_type = other DNA
ggaagagaaccccggacccatgggccccggcgtgc

SEQ ID NO: 20 - DNA, 49 bp, synthetic construct
  source 1..49: mol_type = other DNA
cgacgactccggaacgaattctgattatctgggaggcagggcctgcatg

SEQ ID NO: 21 - DNA, 38 bp, synthetic construct
  source 1..38: mol_type = other DNA
ggaagagaaccccggacccatggccctgccagtgaccg

SEQ ID NO: 22 - DNA, 46 bp, synthetic construct
  source 1..46: mol_type = other DNA
cgacgactccggaacgaattctgatcagcctctgctgtccttccgc

SEQ ID NO: 23 - DNA, 12152 bp, synthetic construct
  source 1..12152: mol_type = other DNA
ttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagctagcgccaccatggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggttggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggctggccgatcctgctggggctgaccggggtccccgcggcgctgcagctccttctgctgcccttcttccccgagagccccaggtacctgctgattcagaagaaagacgaagcggccgccaagaaagccctacagacgctgcgcggctgggactctgtggacagggaggtggccgagatccggcaggaggatgaggcagagaaggccgcgggcttcatctccgtgctgaagctgttccggatgcgctcgctgcgctggcagctgctgtccatcatcgtcctcatgggcggccagcagctgtcgggcgtcaacgctatctactactacgcggaccagatctacctgagcgccggcgtgccggaggagcacgtgcagtacgtgacggccggcaccggggccgtgaacgtggtcatgaccttctgcgccgtgttcgtggtggagctcctgggtcggaggctgctgctgctgctgggcttctccatctgcctcatagcctgctgcgtgctcactgcagctctggcactgcaggacacagtgtcctggatgccatacatcagcatcgtctgtgtcatctcctacgtcataggacatgccctcgggcccagtcccatacccgcgctgctcatcactgagatcttcctgcagtcctctcggccatctgccttcatggtggggggcagtgtgcactggctctccaacttcaccgtgggcttgatcttcccgttcatccaggagggcctcggcccgtacagcttcattgtcttcgccgtgatctgcctcctcaccaccatctacatcttcttgattgtcccggagaccaaggccaagacgttcatagagatcaaccagattttcaccaagatgaataaggtgtctgaagtgtacccggaaaaggaggaactgaaagagcttccacctgtcacttcggaacagggatctggagcaacaaacttctcactactcaaacaagcaggtgacgtggaggagaatcccgggcctatgggctggctgtgttccggcctgctgtttcctgtgtcctgtctggtgctgctgcaggtggccagctccgggaacatgaaagtgctgcaggagcccacatgtgtgtccgactacatgtccatctctacatgtgagtggaagatgaacggccccacaaactgctctaccgagctgcggctgctgtaccagctggtgtttctgctgagcgaggcccacacctgtatcccagaaaataatggcggggccgggtgtgtgtgccacctgctgatggatgacgtggtgtctgccgacaattacaccctggacctgtgggccggacagcagctgctgtggaaggggtccttcaaaccctctgagcacgtgaagccaagggcccccggcaacctgacagtgcacaccaacgtgtctgatacactgctgctgacatggagcaatccataccctcctgacaactacctgtacaaccacctgacctacgccgtgaatatctggagcgaaaatgatcctgccgactttcggatttacaatgtgacctatctggagccctccctgagaattgccgcctctaccctgaaatctggaatctcctaccgcgccagggtgcgggcctgggcccagtgttacaacaccacctggtctgagtggagcccaagcaccaagtggcacaattcttatcgggagccttttgagcagcacctgatcccctggctgggacacctgctggtggggctgtctggcgcctttggcttcatcattctggtgtacctgctgatcaactgtaggaatacaggcccttggctgaagaaggtgctgaagtgtaacacccccgacccctctaagttcttcagccagctgtcctctgaacacgggggagatgtgcagaagtggctgtccagccctttcccatccagctcctttagccccgggggcctggcccctgagatctctccactggaagtgctggagcgggacaaggtgacccagctgctgctgcagcaggacaaggtgccagaacccgcctccctgagctccaaccacagcctgacatcttgctttacaaatcagggatacttcttcttccacctgcccgatgccctggagatcgaggcctgccaggtgtacttcacctacgatccctactctgaggaagacccagatgagggcgtggccggggccccaaccgggtccagcccacagccactgcagccactgtccggcgaagatgacgcctactgcacattcccttccagggatgacctgctgctgttcagcccatctctgctgggcggaccctctcctccaagcacagccccagggggatccggcgccggggaagagaggatgccccctagcctgcaggagcgcgtgcccagagactgggacccccagcccctgggccctccaacccctggggtgcccgacctggtggacttccagcctccacccgagctggtgctgagggaggccggcgaagaggtgcccgacgccggcccccgggagggcgtgtccttcccttggtccagacctccaggacagggcgagttccgcgccctgaacgccaggctgcctctgaacaccgatgcctacctgtctctgcaggaactgcagggccaggacccaacccacctggtgcggagaaagcgcagcggctccggcgagggccggggcagcctgctgacctgcggcgacgtggaagagaaccccggacccatgggccccggcgtgctgctgctgctgctggtggccaccgcctggcacggccagggcggcgaggtgcagcttcaggagtcaggacctggccttgtgaaaccctcacagtcactctccctcacctgttctgtcactggttactccatcactactgattactggggctggatccggaagttcccaggaaataaaatggagtggatgggatacataagctacagtggtagcactggctacaacccatctctcaaaagtcgaatctccattactagagacacatcgaagagtcagttcttcctgcagttgaactctgtaactactgaggacacagccacatattactgtgcaagatacagtagccttgattactggggccgaggagtcatggtcgcagtctcctcaggaggaggttctggcggtggatcaggaggcggttcggatgttgtgatgacccagacaccaccgtctttgtcggttgccattggacaatcagtctccatctcttgcaagtcaagtcagagcctcgtatatagtgatggaaagacatatttgcattggttattacagagtcctggcaggtctccgaagcgcctaatctatcaggtgtctaatctgggctctggagtccctgacaggttcagtggcactggatcacagaaagattttacacttaaaatcagcagagtggaggctgaggatttgggagtttactactgcgcgcaaactacacattttcctctcacgttcggttctgggaccaagctggagatcaaacgggcggccgcaatcgaggtggagcagaagctgatcagcgaggaggacctgctggacaacgagaagagcaacggcaccatcatccacgtgaagggcaagcacctgtgtcccagccctctgttccccggccctagcaagcccttctgggtgctggtggttgtgggcggagtgctggcctgctacagcctgctcgtgaccgtggccttcatcatcttctgggtgagaagcaagagaagcagactgctgcacagcgactacatgaacatgacacccagaagacccggacccaccagaaagcactaccagccctacgctccccctagagacttcgccgcctacagaagcagagtgaagttcagcagaagcgccgacgctcccgcctaccagcaaggccagaaccagctgtacaacgagctgaacctgggcagaagagaggagtacgacgtgctggacaagagaagaggcagagaccctgagatgggaggcaagcccagaagaaagaacccccaggagggcctgtacaacgagctgcagaaggacaagatggccgaggcctacagcgagatcggcatgaagggagagagaagaagaggcaagggccacgacggcctgtaccagggcctgagcaccgccaccaaggacacctacgacgccctgcacatgcaggccctgcctcccagataatcagaattcgttccggagtcgtcgactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcatcttcgccttcgccctcagacgagtcggatctccctttgggccgcctccccgcctggaattaattcgagctcggtacctttaagaccaatgacttacaaggcagctgtagatcttagccactttttaaaagaaaaggggggactggaagggctacgtaactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctcacttgagcgtcgatttttgtgatgctcgtcaggggggcggagcctatggaaaaacgccagcaacgcggcctttttacggttcctggccttttgctggccttttgctcacatg

SEQ ID NO: 24 - DNA, 12512 bp, synthetic construct
  source 1..12512: mol_type = other DNA
ttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagctagcgccaccatggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggttggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggctggccgatcctgctggggctgaccggggtccccgcggcgctgcagctccttctgctgcccttcttccccgagagccccaggtacctgctgattcagaagaaagacgaagcggccgccaagaaagccctacagacgctgcgcggctgggactctgtggacagggaggtggccgagatccggcaggaggatgaggcagagaaggccgcgggcttcatctccgtgctgaagctgttccggatgcgctcgctgcgctggcagctgctgtccatcatcgtcctcatgggcggccagcagctgtcgggcgtcaacgctatctactactacgcggaccagatctacctgagcgccggcgtgccggaggagcacgtgcagtacgtgacggccggcaccggggccgtgaacgtggtcatgaccttctgcgccgtgttcgtggtggagctcctgggtcggaggctgctgctgctgctgggcttctccatctgcctcatagcctgctgcgtgctcactgcagctctggcactgcaggacacagtgtcctggatgccatacatcagcatcgtctgtgtcatctcctacgtcataggacatgccctcgggcccagtcccatacccgcgctgctcatcactgagatcttcctgcagtcctctcggccatctgccttcatggtggggggcagtgtgcactggctctccaacttcaccgtgggcttgatcttcccgttcatccaggagggcctcggcccgtacagcttcattgtcttcgccgtgatctgcctcctcaccaccatctacatcttcttgattgtcccggagaccaaggccaagacgttcatagagatcaaccagattttcaccaagatgaataaggtgtctgaagtgtacccggaaaaggaggaactgaaagagcttccacctgtcacttcggaacagggatctggagcaacaaacttctcactactcaaacaagcaggtgacgtggaggagaatcccgggcctatgggctggctgtgttccggcctgctgtttcctgtgtcctgtctggtgctgctgcaggtggccagctccgggaacatgaaagtgctgcaggagcccacatgtgtgtccgactacatgtccatctctacatgtgagtggaagatgaacggccccacaaactgctctaccgagctgcggctgctgtaccagctggtgtttctgctgagcgaggcccacacctgtatcccagaaaataatggcggggccgggtgtgtgtgccacctgctgatggatgacgtggtgtctgccgacaattacaccctggacctgtgggccggacagcagctgctgtggaaggggtccttcaaaccctctgagcacgtgaagccaagggcccccggcaacctgacagtgcacaccaacgtgtctgatacactgctgctgacatggagcaatccataccctcctgacaactacctgtacaaccacctgacctacgccgtgaatatctggagcgaaaatgatcctgccgactttcggatttacaatgtgacctatctggagccctccctgagaattgccgcctctaccctgaaatctggaatctcctaccgcgccagggtgcgggcctgggcccagtgttacaacaccacctggtctgagtggagcccaagcaccaagtggcacaattcttatcgggagccttttgagcagcacctgatcccctggctgggacacctgctggtggggctgtctggcgcctttggcttcatcattctggtgtacctgctgatcaactgtaggaatacaggcccttggctgaagaaggtgctgaagtgtaacacccccgacccctctaagttcttcagccagctgtcctctgaacacgggggagatgtgcagaagtggctgtccagccctttcccatccagctcctttagccccgggggcctggcccctgagatctctccactggaagtgctggagcgggacaaggtgacccagctgctgctgcagcaggacaaggtgccagaacccgcctccctgagctccaaccacagcctgacatcttgctttacaaatcagggatacttcttcttccacctgcccgatgccctggagatcgaggcctgccaggtgtacttcacctacgatccctactctgaggaagacccagatgagggcgtggccggggccccaaccgggtccagcccacagccactgcagccactgtccggcgaagatgacgcctactgcacattcccttccagggatgacctgctgctgttcagcccatctctgctgggcggaccctctcctccaagcacagccccagggggatccggcgccggggaagagaggatgccccctagcctgcaggagcgcgtgcccagagactgggacccccagcccctgggccctccaacccctggggtgcccgacctggtggacttccagcctccacccgagctggtgctgagggaggccggcgaagaggtgcccgacgccggcccccgggagggcgtgtccttcccttggtccagacctccaggacagggcgagttccgcgccctgaacgccaggctgcctctgaacaccgatgcctacctgtctctgcaggaactgcagggccaggacccaacccacctggtgcggagaaagcgcagcggctccggcgagggccggggcagcctgctgacctgcggcgacgtggaagagaaccccggacccatggccctgccagtgaccgccctgctgctgccactggccctgctgctgcacgcagcacggccaaagaatgaggtggagcagagcccacagaacctgaccgcacaggagggagagttcatcacaatcaattgctcttacagcgtgggaatctccgccctgcactggctgcagcagcacccaggaggaggcatcgtgtctctgtttatgctgagctccggcaagaagaagcacggcaggctgatcgccaccatcaacatccaggagaagcactctagcctgcacatcacagcctcccaccctcgcgattctgccgtgtatatctgtgcagcaagcctgatccagggagcacagaagctggtgttcggacagggcaccaggctgacaatcaatcccccttacatccagaaccctgacccagccgtgtatcagctgcgcgactctaagtcctctgataagagcgtgtgcctgttcaccgactttgattctcagacaaacgtgagccagtctaaggacagcgacgtgtacatcaccgacaagtgcgtgctggatatgcggtccatggactttaagagcaattccgccgtggcctggagcaacaagtccgatttcgcctgcgccaacgcctttaacaattctatcatccctgaggacacattctttcccagctccgacgtgccttgtgatgtgaagctggtggagaagtccttcgagaccgacacaaatctgaactttcagaatctgctggtcatcgtgctgagaatcctgctgctgaaggtggtgggcttcaacctgctgatgaccctgaggctgtggtctagcggctccggagagggaagaggctctctgctgacctgcggcgatgtggaggagaatccaggacctatggcactgcctgtgacagccctgctgctgcctctggccctgctgctgcatgcagcaaggcctgacgcaggcgtgatccagagcccaaggcacgaggtgaccgagatgggacaggaggtgacactgaggtgtaagccaatctctggccacaatagcctgttctggtacaggcagaccatgatgcgcggcctggagctgctgatctacttcaacaataacgtgcccatcgacgatagcggcatgcctgaggatcggttctccgccaagatgcccaacgcctcttttagcacactgaagatccagcctagcgagccaagagactccgccgtgtacttctgcgcctcctctcccggcggcaatgagcagttctttggccctggcaccaggctgacagtgctggatctgaagaacgtgttcccacccgaggtggccgtgtttgagccatccaaggccgagatcgcccacacccagaaggccaccctggtgtgcctggcaaccggcttctatcccgaccacgtggagctgtcctggtgggtgaatggcaaggaggtgcactctggcgtgtgcacagatccacagcccctgaaggagcagcctgccctgaacgacagccgctactgtctgagctcccggctgagagtgtccgccaccttttggcagaatccaaggaaccacttccgctgccaggtgcagttttatggcctgagcgagaacgatgagtggacccaggacagggcaaagccagtgacacagatcgtgtccgccgaggcatggggaagagcagattgtggcatcacctccgcctcttatcaccagggcgtgctgagcgccaccatcctgtacgagatcctgctgggcaaggccacactgtatgccgtgctggtgtctgccctggtgctgatggccatggtgaagcggaaggacagcagaggctgatcagaattcgttccggagtcgtcgactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcatcttcgccttcgccctcagacgagtcggatctccctttgggccgcctccccgcctggaattaattcgagctcggtacctttaagaccaatgacttacaaggcagctgtagatcttagccactttttaaaagaaaaggggggactggaagggctacgtaactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctcacttgagcgtcgatttttgtgatgctcgtcaggggggcggagcctatggaaaaacgccagcaacgcggcctttttacggttcctggccttttgctggccttttgctcacatg

(19) United States
(12) Patent Application Publication
(10) Pub. No.: US 2025/0049917 A1
(43) Pub. Date: Feb. 13, 2025

(54) ENGINEERED IMMUNE CELL

(71) Applicant: KINGS COLLEGE LONDON, London (GB)

(72) Inventors: Sophie PAPA, London (GB); Robert PAGE, London (GB); Esperanza PERUCHA, London (GB)

(21) Appl. No.: 18/719,622

(22) PCT Filed: Dec. 14, 2022
(86) PCT No.: PCT/EP2022/085980

§ 371 Date: Jun. 13, 2024

(30) Foreign Application Priority Data
Dec. 15, 2021 (GB) 2118201.9

(51) Int. Cl. A61K 39/00; A61P 35/00; C07K 14/47; C12N 5/0783; C12N 9/12; C12N 9/24; C12N 9/78; C12N 9/88

(52) U.S. Cl. CPC A61K 39/4611; A61K 39/4631; A61P 35/00; C07K 14/47; C12N 5/0636; C12N 9/1205; C12N 9/2402; C12N 9/78; C12N 9/88; C12Y 207/01003; C12Y 302/01028; C12Y 305/04004; C12Y 401/02013; C12N 2510/00

(57) ABSTRACT

An immune cell that is genetically engineered to express an exogenous alternative carbon source (ACS) metabolism gene, in which the ACS is not glucose and wherein the ability of the immune cell to metabolise the ACS is increased due to expression of the exogenous ACS metabolism gene. Also provided are polynucleotides, vectors, pharmaceutical compositions, methods of genetically engineering the immune cell and methods of use in therapy.

### TECHNICAL FIELD

This invention relates to an immune cell which is genetically engineered to express an exogenous alternative carbon source (ACS) metabolism gene, wherein the ACS is not glucose and wherein the ability of the immune cell to metabolise the ACS is increased due to expression of the exogenous ACS metabolism gene. Also provided are polynucleotides, vectors, pharmaceutical compositions, methods of genetically engineering the immune cell and methods of use in therapy.

### BACKGROUND

The treatment of cancer using immunotherapy is becoming increasingly common. Immunotherapy using chimeric antigen receptor (CAR)-engineered T-cells has delivered significant clinical benefit for patients with haematological malignancies. Despite this, the efficacy of CAR-engineered T-cells against solid tumours is sub-optimal.

Solid tumour microenvironments (TME) are known to be immunosuppressive. By suppressing the immune system, the cancer can escape immune control, enabling continued tumour growth. This immunosuppressive nature can hinder immunotherapeutic efficacy.

An underappreciated immunosuppressive mechanism of the tumour environment is metabolic competition between cells of the tumour environment for essential metabolites. In particular, there is metabolic competition between the tumour cells and CAR T-cells for glucose. This is critical because immunotherapeutic cells, such as T cells, are highly reliant on glucose as a carbon source. The anaerobic glycolysis of glucose by the T-cells is necessary to provide the energy and metabolic intermediates to sustain their anti-tumour effector function. Thus, in established solid tumours, tumour cells can outcompete therapeutic T-cells.

There remains a need to improve the efficacy of immunotherapy, especially in the treatment of cancers such as solid tumour cancers. In particular, there remains a need to improve the efficacy of therapeutic T-cells. The present invention seeks to address one or more of the aforementioned issues.

### SUMMARY OF THE INVENTION

The present invention provides an immune cell which is genetically engineered to express an exogenous alternative carbon source (ACS) metabolism gene, wherein the ACS is not glucose. The ability of the immune cell to metabolise the ACS is increased due to expression of the exogenous ACS metabolism gene.

In the context of the present invention, the term “exogenous” refers to a gene which has been introduced into the immune cell. As the skilled person will appreciate, “genetically engineered” refers to the introduction of a polynucleotide encoding the exogenous ACS metabolism gene into the cell. Typically, the polynucleotide is recombinant.

The ACS will be understood to be an alternative carbon source to glucose. Prior to being genetically engineered, the immune cell is unable to metabolise the ACS, or is able to metabolise the ACS at a lower level.

By “express an exogenous ACS metabolism gene”, this will be understood to refer to the production of an amount of the protein product encoded by the ACS metabolism gene. In the context of the present invention, expression comprises at least translation and preferably also transcription of the gene to synthesize the product. As such, in the context of the present invention, “express an exogenous ACS metabolism gene” may comprise the transcription of exogenous DNA encoding the ACS metabolism gene into mRNA then translation of the mRNA into an amount of the protein product encoded by the ACS metabolism gene. In such instances, the skilled person may use mRNA expression levels as a marker of protein expression. Alternatively, “express an exogenous ACS metabolism gene” may comprise the translation of exogenous RNA encoding the ACS metabolism gene into an amount of the protein product encoded by the ACS metabolism gene.

Expression of the exogenous ACS gene by the genetically engineered immune cell may comprise transcription and/or translation of the exogenous ACS metabolism gene. Expression by the genetically engineered immune cell preferably comprises transcription and translation of the exogenous ACS metabolism gene. Expression may comprise a detectable amount of mRNA and/or protein encoded by the exogenous ACS metabolism gene in the genetically engineered immune cell. Expression preferably comprises a detectable amount of mRNA and protein encoded by the exogenous ACS metabolism gene in the genetically engineered immune cell. Methods for measuring the presence of mRNA and/or protein are known in the art and discussed in more detail below. The mRNA and/or protein may be present in any amount in the genetically engineered immune cell. Amounts of the mRNA and/or protein are discussed in more detail below. In the context of the present invention, the terms “amount” and “level” are interchangeable.

Prior to being genetically engineered, expression of the exogenous ACS gene may be undetectable. In some embodiments, prior to being genetically engineered, expression of the exogenous ACS gene comprises an undetectable amount of mRNA and/or protein encoded by the exogenous ACS metabolism gene in the cell. Prior to being genetically engineered, expression of the exogenous ACS gene may comprise an undetectable amount of mRNA and protein encoded by the exogenous ACS metabolism gene in the cell.

Thus, genetic engineering of the immune cell increases and/or initiates expression of the ACS metabolism gene in the immune cell. The expression and/or detectable amount of the exogenous ACS gene in the genetically engineered immune cell is therefore increased.

The expression and/or detectable amount of the exogenous ACS gene in the genetically engineered immune cell may be increased by at least about 10%, at least about 20%, at least about 30%, at least about 40%, at least about 50%, at least about 60%, at least about 70%, at least about 80%, at least about 90%, at least about 95% or at least about 99%. In some embodiments, the expression and/or detectable amount of the exogenous ACS gene in the genetically engineered immune cell is increased by at least about 50%. Preferably, the expression and/or detectable amount is increased by at least about 70%, more preferably at least about 80%, most preferably at least about 90%.

The expression and/or detectable amount of the exogenous ACS gene in the genetically engineered immune cell may be increased at least about one fold, at least about two fold, at least about three fold, at least about four fold, at least about five fold, at least about six fold, at least about seven fold, at least about eight fold, at least about nine fold, at least about 10 fold, at least about 12 fold, at least about 15 fold, at least about 20 fold, at least about 50 fold, at least about 100 fold, at least about 500 fold, at least about 1000 fold, at least about 2000 fold, at least about 5000 fold or at least about 10000 fold.

The expression and/or detectable amount of the mRNA and/or protein encoded by the exogenous ACS gene in the genetically engineered immune cell may be increased.

The expression and/or detectable amount of mRNA encoded by the exogenous ACS gene in the genetically engineered immune cell may be increased by at least about 10%, at least about 20%, at least about 30%, at least about 40%, at least about 50%, at least about 60%, at least about 70%, at least about 80%, at least about 90%, at least about 95% or at least about 99%. In some embodiments, the expression and/or detectable amount of mRNA encoded by the exogenous ACS gene in the genetically engineered immune cell is increased by at least about 50%. Preferably, the expression and/or detectable amount of mRNA encoded by the exogenous ACS gene in the genetically engineered immune cell is increased by at least about 70%, more preferably at least about 80%, most preferably at least about 90%.

The expression and/or detectable amount of mRNA encoded by the exogenous ACS gene in the genetically engineered immune cell may be increased at least about one fold, at least about two fold, at least about three fold, at least about four fold, at least about five fold, at least about six fold, at least about seven fold, at least about eight fold, at least about nine fold, at least about 10 fold, at least about 12 fold, at least about 15 fold, at least about 20 fold, at least about 50 fold, at least about 100 fold, at least about 500 fold, at least about 1000 fold, at least about 2000 fold, at least about 5000 fold or at least about 10000 fold.

The expression and/or detectable amount of protein encoded by the exogenous ACS gene in the genetically engineered immune cell may be increased by at least about 10%, at least about 20%, at least about 30%, at least about 40%, at least about 50%, at least about 60%, at least about 70%, at least about 80%, at least about 90%, at least about 95% or at least about 99%. In some embodiments, the expression and/or detectable amount of protein encoded by the exogenous ACS gene in the genetically engineered immune cell is increased by at least about 50%. Preferably, the expression and/or detectable amount of protein is increased by at least about 70%, more preferably at least about 80%, most preferably at least about 90%.

The expression and/or detectable amount of protein encoded by the exogenous ACS gene in the genetically engineered immune cell may be increased at least about one fold, at least about two fold, at least about three fold, at least about four fold, at least about five fold, at least about six fold, at least about seven fold, at least about eight fold, at least about nine fold, at least about 10 fold, at least about 12 fold, at least about 15 fold, at least about 20 fold, at least about 50 fold, at least about 100 fold, at least about 500 fold, at least about 1000 fold, at least about 2000 fold, at least about 5000 fold or at least about 10000 fold.

Various methods are known in the art to detect proteins including, for example, western blots, flow cytometry and ELISAs. Normalisation of protein expression may be to a housekeeping protein product. The skilled person will be aware of suitable housekeeping genes and products. The expression and/or detectable amount of protein preferably comprises a normalised value.

The expression and/or detectable amount of protein may be detected using flow cytometry.

Normalisation of protein expression may be to an isotype control antibody. The expression and/or detectable amount of protein detected using flow cytometry may be quantified by population shift and/or mean fluorescence intensity.

The expression and/or detectable amount of mRNA can be detected using RNA-seq methods. Various RNA-seq methods are commercially available and known to those skilled in the art. RNA-seq methods function by mapping the number of RNA reads aligned to each gene (in this instance, the ACS metabolism gene) under each biological condition, to obtain a read count. The reads can then be normalised to provide a normalised read count.

The expression and/or detectable amount of mRNA preferably comprises a normalised read count. Thus, the genetically engineered cell may have an exogenous ACS metabolism gene normalised read count.

Normalisation of RNA may be by library size. Normalisation of RNA by library size may comprise dividing each read count by the total number of reads in its sample. This may otherwise be referred to as a Total Count Normalisation or Reads per kilobase per million mapped reads (RPKM) method. In other embodiments, normalisation of RNA by library size comprises dividing each read count by the transcript length. This may otherwise be referred to as transcripts per million (TPM).

Alternatively, normalisation may be by distribution/testing. This comprises determining expression levels of other known non-differentially expressed genes and determining the distribution across the samples. The non-differentially expressed genes may comprise housekeeping genes, of which various genes are known in the art.

The genetically engineered cell preferably has a normalised read count (of the exogenous ACS metabolism gene) of at least about 3000, at least about 3500, at least about 4000, at least about 5000, at least about 6000, at least about 7000, at least about 8000, at least about 9000, at least about 10000, at least about 15000 or at least about 20000. In some embodiments the genetically engineered cell has a normalised read count of at least about 8000, optionally at least about 10000.

Prior to being genetically engineered, the cell preferably has a normalised read count (of the exogenous ACS metabolism gene) of less than about 2500, less than about 2000, less than about 1500, less than about 1000, less than about 900, less than about 800, less than about 700, less than about 600, less than about 500, less than about 400, less than about 300, less than about 200, less than about 100, less than about 50, less than about 40, less than about 30, less than about 20, less than about 10, less than about 8, less than about 5, less than about 4, less than about 3, less than about 2, less than about 1, less than about 0.5, less than about 0.1 or less than about 0.01. More preferably, prior to being genetically engineered, the cell has a normalised read count of less than about 5, less than about 4, less than about 3, less than about 2, less than about 1, less than about 0.5, less than about 0.1 or less than about 0.01. Most preferably, prior to being genetically engineered, the cell has a normalised read count of less than about 0.1 or less than about 0.01.

The normalised read count may be transformed to a log value, for example to log 2 counts per million. Thus, in some embodiments, the expression and/or detectable amount of mRNA comprises a log value, for example log 2 counts per million.

The genetically engineered cell preferably has a log value (of the exogenous ACS metabolism gene) of at least about 1.3, at least about 1.4, at least about 1.5, at least about 1.6, at least about 1.7, at least about 1.8, at least about 1.9, at least about 2 or at least about 3. In some embodiments the genetically engineered cell has a log value of at least about 2.

Prior to being genetically engineered, the cell preferably has a log value (of the exogenous ACS metabolism gene) of less than about 1.2, less than about 1.1, less than about 1, less than about 0.9, less than about 0.8, less than about 0.7, less than about 0.6, less than about 0.5, less than about 0.4, less than about 0.3, less than about 0.2, less than about 0.1, less than about 0.09, less than about 0.08, less than about 0.07, less than about 0.06, less than about 0.05, less than about 0.04, less than about 0.03, less than about 0.02, less than about 0.01, less than about 0.009, less than about 0.008, less than about 0.007, less than about 0.006, less than about 0.005, less than about 0.004, less than about 0.003, less than about 0.002 or less than about 0.001. In some embodiments, prior to being genetically engineered, the cell has a log value of less than about 0.1, less than about 0.09, less than about 0.08, less than about 0.07, less than about 0.06, less than about 0.05, less than about 0.04, less than about 0.03, less than about 0.02, less than about 0.01, less than about 0.009, less than about 0.008, less than about 0.007, less than about 0.006, less than about 0.005, less than about 0.004, less than about 0.003, less than about 0.002 or less than about 0.001. Preferably, prior to being genetically engineered, the cell has a log value of less than about 0.05, less than about 0.04, less than about 0.03, less than about 0.02, less than about 0.01, less than about 0.009, less than about 0.008, less than about 0.007, less than about 0.006, less than about 0.005, less than about 0.004, less than about 0.003, less than about 0.002 or less than about 0.001. More preferably, prior to being genetically engineered, the cell has a log value of less than about 0.02, less than about 0.01, less than about 0.009, less than about 0.008, less than about 0.007, less than about 0.006, less than about 0.005, less than about 0.004, less than about 0.003, less than about 0.002 or less than about 0.001.

When the expression of the exogenous ACS metabolism gene in the immune cell is undetectable prior to genetic engineering, the skilled person will appreciate that any expression of the exogenous ACS metabolism gene in the genetically engineered immune cell is an increase. It will, however, be appreciated that in such embodiments, the increase cannot be quantified as a percentage or fold increase since the starting value is undetectable.

As the skilled person will appreciate, a primary carbon source for immune cells is glucose. In glucose-depleted environments, immune cells may struggle to obtain energy and thus have reduced or no effector function. Advantageously, the expression of an exogenous ACS metabolism gene in the immune cell allows the immune cell to utilise other sources of carbon which it cannot or is less able to utilise in its wild-type, non-engineered form. This is particularly beneficial, for example, in a TME, where metabolic competition can deplete glucose sources.

Without wishing to be bound by theory, the inventors believe that the expression of an exogenous ACS metabolism gene enables the immune cell to have increased effector activity and/or retain effector activity for an increased period of time, as opposed to immune cells which have not been engineered to express the exogenous ACS metabolism gene. Expression of an exogenous ACS metabolism gene may also enable the immune cell to become active in the tumour microenvironment. For non-engineered immune cells, the tumour suppressive environment may prevent activation.

Thus, in some embodiments, the genetically engineered immune cell has increased effector activity and/or retains effector activity for an increased period of time. In some embodiments, the genetically engineered immune cell has increased effector activity and retains effector activity for an increased period of time. It will be appreciated that the increased effector activity and/or retention of effector activity for an increased period of time is relative to the immune cell prior to being genetically engineered.

In some embodiments, the genetically engineered immune cell has increased effector activity. Effector activity may comprise cytotoxic activity, for example against tumour cells. Thus, increased effector activity may comprise or consist of increased cytotoxic activity.

Cytotoxic activity may be increased by at least about 10%, at least about 20%, at least about 30%, at least about 40%, at least about 50%, at least about 60%, at least about 70%, at least about 80%, at least about 90%, at least about 95% or at least about 99%. In some embodiments, the cytotoxic activity of the genetically engineered immune cell is increased by at least about 50%. Preferably, the cytotoxic activity of the genetically engineered immune cell is increased by at least about 70%, more preferably at least about 80%, most preferably at least about 90%.

The cytotoxic activity of an immune cell can be determined by in vitro co-incubation of the immune cell with tumour cells. The resulting viability of the tumour cells can be a measure of cytotoxic activity of the immune cell. A monolayer may comprise the tumour cells. This may otherwise be referred to as a cytotoxicity assay. Increased cytotoxic activity may thus comprise decreased viability of the tumour cells. The viability of the tumour cells may be decreased by at least about 10%, at least about 20%, at least about 30%, at least about 40%, at least about 50%, at least about 60%, at least about 70%, at least about 80% or at least about 90%.

In some embodiments, the ratio of immune cells to tumour cells in a cytotoxicity assay is less than 1:10, 1:9, 1:8, 1:7, 1:6, 1:5, 1:4, 1:3, or 1:2. The immune cells and tumour cells in the cytotoxicity assay may be co-incubated for at least about 12 hours, at least about 16 hours, at least about 20 hours, at least about 24 hours, at least about 48 hours or at least about 72 hours.

Prior to being genetically engineered, the immune cell may have decreased effector activity and/or retains effector activity for a decreased period of time (relative to the genetically engineered immune cell). Prior to being genetically engineered, cytotoxic activity of the immune cell may be decreased by at least about 10%, at least about 20%, at least about 30%, at least about 40%, at least about 50%, at least about 60%, at least about 70%, at least about 80%, at least about 90%, at least about 95% or at least about 99%. In some embodiments, the cytotoxic activity of the immune cell prior to being genetically engineered is decreased by at least about 50%. Preferably, the cytotoxic activity of the immune cell prior to being genetically engineered is decreased by at least about 70%, more preferably at least about 80%, most preferably at least about 90%.

By “ACS metabolism gene”, this will be understood to refer to a gene, the expression of which enables the metabolism of the alternative carbon source in the immune cell. In the context of the present invention, the ACS metabolism gene is typically in a pathway for the metabolism of the alternative carbon source in other cells which are naturally able to metabolise the ACS. Such genes may include, but not necessarily be limited to, genes encoding transporter proteins and enzymes, for example catabolic enzymes. In some embodiments, the exogenous ACS metabolism gene encodes a transporter protein or enzyme.

Transporter proteins may otherwise be referred to as membrane transport proteins or transporters. The transporter proteins of the invention are proteins which can transport a carbon source other than glucose across the cellular membrane. In the context of the present invention, this will be understood to be transport from the extracellular to the intracellular environment.

By enzyme, this will be understood to refer to an enzyme which can metabolise the ACS. The enzyme may be secreted from the cell. In the extracellular environment, the enzyme may be capable of breaking down the ACS into a monosaccharide which the immune cell is naturally capable of transporting across the cell membrane. Alternatively, the enzyme may not be secreted and/or may function intracellularly, to enable break down of the ACS after it has crossed the cell membrane into the cell. In some embodiments, the enzyme is an extracellular membrane bound enzyme or a transmembrane enzyme. Preferably, the enzyme is an extracellular membrane bound enzyme.

Various alternative carbon sources are suitable for the present invention. In some embodiments, the ACS is a monosaccharide, nucleoside or disaccharide. The monosaccharide may comprise or consist of fructose.

As the skilled person will appreciate, a nucleoside consists of a five-carbon sugar, preferably ribose, and a nucleobase. The nucleoside may comprise or consist of cytidine, uridine, guanosine, inosine, thymidine, adenosine or combinations thereof. Preferably, the nucleoside is selected from guanosine and adenosine.

In some embodiments, the ACS is fructose or adenosine.

In some embodiments, the ACS is fructose. Alternatively, the ACS may be adenosine. Adenosine is known to inhibit T cell activation and is produced at high levels in the TME. The present inventors have found that expression of an exogenous adenosine metabolism gene, preferably encoding an adenosine-metabolising enzyme, in an immune cell enables the cell to generate inosine, which the immune cell can use as a carbon source.

Suitable disaccharides include, but are not necessarily limited to sucrose, lactose, maltose, trehalose, cellobiose, chitobiose, kojibiose, nigerose, isomaltose, αβ-trehalose, ββ-trehalose, sophorose, laminaribiose, gentiobiose, trehalulose, turanose, maltulose, leucrose, isomaltulose, gentiobiulose, mannobiose, melibiose, melibiulose, rutinose, rutinulose and xylobiose. In some embodiments, the disaccharide is selected from sucrose, lactose, maltose, isomaltose and trehalose.

In some embodiments, the disaccharide is trehalose. Trehalose may comprise or consist of αα-trehalose, αβ-trehalose, ββ-trehalose, or a combination thereof. In some embodiments, the disaccharide is αα-trehalose.

In some embodiments, the ACS is trehalose, fructose or adenosine. The ACS may be trehalose or adenosine. Preferably, the ACS is trehalose or fructose. More preferably, the ACS is trehalose.

In some embodiments, the exogenous ACS metabolism gene encodes a disaccharide transporter or enzyme. Typically, monosaccharides and disaccharides cannot cross the cell membrane unless they are transported across the membrane using a specific membrane transporter protein. In some instances, disaccharides are first broken down into monosaccharides by an extracellular enzyme. The monosaccharide may then be transported across the cell membrane into the cell by a membrane transporter protein specific for the monosaccharide. Therefore, unless the appropriate membrane transport protein or enzyme is endogenously expressed by the cell, the particular ACS cannot be metabolised by the cell unless it is genetically engineered to express an exogenous ACS metabolism gene.

Optionally, the exogenous ACS metabolism gene does not encode a trehalose transporter, for example TRET1.

In some embodiments, the exogenous ACS metabolism gene encodes a monosaccharide transporter. For example, the exogenous ACS metabolism gene may encode a fructose transporter such as GLUT5. Thus, in some embodiments, the exogenous ACS metabolism gene encodes GLUT5. The expression of GLUT5 enables the transport of fructose across the cell membrane. Once in the cell, fructose can be metabolised by endogenous enzymes. Typically, endogenous levels of GLUT5 in human T-cells, NK cells and monocytes are undetectable, for example a normalised read count of less than 1.

In some embodiments, the genetically engineered immune cell does not comprise the genetically engineered immune cells of WO 2020/010110.

In some embodiments, the exogenous ACS metabolism gene encodes an enzyme. In some embodiments, the exogenous ACS metabolism gene encodes a disaccharide or nucleoside-metabolising enzyme. The disaccharide may be lactose, maltose, isomaltose, sucrose, trehalose or combinations thereof. In some embodiments, the ACS metabolism gene encodes an enzyme selected from LCT, MGAM, SI, Ketohexokinase (KHK), Aldolase B (ALDOB) and trehalase. LCT is an enzyme which can metabolise lactose into galactose and glucose. MGAM can metabolise maltose into glucose. SI can metabolise isomaltose into glucose, and metabolise sucrose into glucose and fructose. Trehalase can metabolise trehalose into glucose. KHK and ALDOB are intracellular, in particular cytoplasmic, enzymes. LCT, MGAM and SI are transmembrane enzymes, wherein the enzyme comprises an extracellular enzyme domain, a transmembrane domain, and an intracellular domain. Trehalase is an extracellular membrane bound enzyme; trehalase is secreted by the cell but retained on the cell membrane by a GPI-anchor.

For example, the exogenous ACS metabolism gene may encode a trehalose or nucleoside-metabolising enzyme. In some embodiments, the exogenous ACS metabolism gene encodes a trehalose or adenosine-metabolising enzyme. Exemplary adenosine-metabolising enzymes include ADA and ADA2. ADA and ADA2 have adenoside deaminase activity, which function to deaminate adenosine to inosine, once secreted from the cell. Advantageously, the exogenous expression of an adenosine enzyme such as ADA2 in an immune cell may simultaneously prevent adenosine-mediated immunosuppression and generate inosine for use by the immune cell as an alternative carbon source to glucose.

In some embodiments, the exogenous ACS metabolism gene encodes an enzyme selected from ADA, ADA2, LCT, MGAM, SI, Ketohexokinase (KHK), Aldolase B (ALDOB) and trehalase.

In some embodiments, the exogenous ACS metabolism gene encodes an enzyme selected from ADA2, Ketohexokinase (KHK), Aldolase B (ALDOB) and trehalase.

Preferably, the exogenous ACS metabolism gene encodes an enzyme selected from Ketohexokinase (KHK), Aldolase B (ALDOB) and trehalase.

Most preferably, the exogenous ACS metabolism gene encodes the enzyme trehalase. Trehalase can be secreted from cells into the extracellular environment, where it can catalyse the breakdown of the disaccharide trehalose into the monosaccharide glucose. The glucose can then be transported across the cell membrane into the cell by endogenous glucose transporters. Endogenous trehalase is undetectable in human and murine immune cells. The normalised read count of the gene encoding trehalase in non-engineered human immune cells is typically about 0. This includes neutrophils, monocytes, B cells, CD4 T cells, CD8 T cells and NK cells.

Optionally, the exogenous ACS metabolism gene encodes LCT, MGAM, SI, Ketohexokinase (KHK), Aldolase B (ALDOB), trehalase or GLUT5.

The exogenous ACS metabolism gene may encode ADA2, GLUT5 or trehalase. In some embodiments, the exogenous ACS metabolism gene encodes ADA2 or trehalase.

In some embodiments, the exogenous ACS metabolism gene encodes trehalase or GLUT5. In some embodiments, the exogenous ACS metabolism gene does not encode ADA. In some embodiments the exogenous ACS metabolism gene does not encode ADA2. The exogenous ACS metabolism gene may not encode ADA and ADA2.

The exogenous ACS metabolism gene may be a wild-type gene or a variant thereof. In some embodiments, the exogenous ACS metabolism gene is a mammalian gene or a variant thereof. For example, the ACS metabolism gene may be a human, dog, cat, horse, bovine, murine, porcine or ovine gene, or a variant thereof. In some embodiments, the ACS metabolism gene is a human, mouse or rat gene, or variant thereof. Preferably, the ACS metabolism gene is a human gene or variant thereof.

As used herein, the term “variant” in the context of a gene encompasses a nucleotide sequence which is a naturally occurring polymorphic form of the basic sequence as well as synthetic variants, in which one or more nucleotides within the sequence are inserted, removed or replaced. The variant may otherwise be referred to as a functional variant, in that while one or more of the nucleotides within the chain are inserted, removed, or replaced, relative to the basic sequence, the protein encoded by the variant substantially retains the functional activity of the protein encoded by the basic sequence. “Substantially retains” will be understood to refer to a functional activity of at least about 80%, at least about 85%, at least about 90%, at least about 95%, at least about 97%, at least about 98%, at least about 99% or at least about 100% of the protein encoded by the basic sequence. A variant of the present invention may have a functional activity equivalent or improved to the basic sequence. Functional variants also encompass truncated versions of the gene. Truncated versions of the gene are shortened versions of the basic nucleotide sequence which produces a biological effect in the translated protein which is equivalent to or improved relative to the protein encoded by the basic sequence.

In some embodiments, the immune cell comprises a population of immune cells. Any number of cells may be present in the population. The population preferably comprises at least about 5×10⁵ immune cells. The population more preferably comprises at least about 1×10⁶, at least about 2×10⁶, at least about 2.5×10⁶, at least about 5×10⁶, at least about 1×10⁷, at least about 2×10⁷, at least about 5×10⁷, at least about 1×10⁸ or at least about 2×10⁸ immune cells. In some instances, the population may comprise at least about 1.0×10⁷, at least about 1.0×10⁸, at least about 1.0×10⁹, at least about 1.0×10¹⁰, at least about 1.0×10¹¹ or at about least 1.0×10¹² immune cells or even more.

Any immune cell is suitable for the present invention. For example, the immune cell may comprise a leukocyte such as a neutrophil, eosinophil, mast cell, basophil, monocyte, lymphocyte or combinations thereof.

The immune cell may comprise or originate from a peripheral blood mononuclear cell (PBMC). The immune cell may comprise a lymphocyte and/or monocyte.

Monocytes may be selected from dendritic cells and macrophages. Lymphocytes encompass T-cells, B-cells, Natural Killer (NK) cells and NKT cells. For example, the cell may comprise a T-cell, natural killer (NK) cell, NK cell, neutrophil, or B-cell.

In some embodiments, the immune cell is selected from a B-cell, T-cell and Natural Killer (NK) cell. The immune cell may be a T-cell.

The T cell may be a CD4⁺ T-cell. Alternatively, the T-cell may be a CD8⁺ T-cell. The T-cell may be a αβ T-cell or a γδ T-cell. In some embodiments, the immune cell comprises an NKT cell.

In some embodiments, the genetically engineered immune cell comprises a genetically engineered regulatory immune cell. By regulatory immune cell, this will be understood to refer to an immune cell which is capable of downregulating an inflammatory response, for example by the secretion of regulatory cytokines such as IL-10 and/or TGF-β. Such regulatory immune cells may have particular utility in the treatment of allergic and/or autoimmune disease. Regulatory immune cells may also have utility in the treatment and/or prevention of transplant rejection. Optionally, the T cell is a regulatory T cell (Treg). In some embodiments, the Treg is FoxP3 and/or CD25⁺.

The immune cell may comprise a stem cell, for example an embryonic stem cell, induced pluripotent stem cell and/or mesenchymal stem cell. In such embodiments, it will be appreciated that the stem cell may be differentiated in vitro into a particular cell type before or after being genetically engineered to express the exogenous ACS metabolism gene.

The immune cell may be a eukaryotic immune cell. For example, the immune cell may be a mammalian immune cell, such as a human, dog, cat, horse, bovine, murine, porcine or ovine cell. In some embodiments, the immune cell is a human immune cell. In some embodiments, the immune cell is a murine immune cell, optionally a mouse immune cell.

The immune cell may further express an exogenous receptor or ligand. In the context of the present invention, an exogenous receptor or ligand will be understood to refer to a receptor or ligand which has been introduced into the immune cell by genetic engineering. Exemplary receptors may include T cell receptors, chimeric antigen receptors (CARs), chemokine receptors and cytokine receptors. Exemplary ligands may include cytokines, chemokines, or other suitable ligands. The ligand may be a homing molecule.

In some embodiments, the immune cell further expresses an exogenous TCR and/or a chimeric antigen receptor (CAR). Preferably, the exogenous TCR is a variant of a wild type TCR or a chimeric TCR. The term “chimeric TCR”, as used herein, will be understood to refer to a non-naturally occurring TCR, wherein the TCR comprises polypeptides or polypeptide fragments from two or more different organisms. For example, a chimeric TCR may comprises polypeptide chains from a mouse and a human TCR.

In some embodiments, the immune cell is a PBMC expressing an exogenous TCR and/or a CAR. In some embodiments, the immune cell is a human PBMC expressing an exogenous TCR and/or a CAR.

In some embodiments, the immune cell is a T-cell expressing an exogenous TCR and/or a CAR. In some embodiments, the immune cell is a human T-cell expressing an exogenous TCR and/or a CAR.

Thus, the immune cell may be a CAR cell. Preferably, the immune cell is a CAR T-cell, CAR B-cell or CAR NK cell. In some embodiments, the immune cell is a CAR T-cell.

As the skilled person will appreciate, CARs comprise artificial antigen receptors. As such, cells expressing CARs have been genetically engineered to express the CAR.

Suitable CARs are known to those skilled in the art. In particular, the CAR may comprise or consist of a first, second, third, or fourth generation CAR.

First generation CARs comprise or consist of an extracellular binding domain, a transmembrane domain, and one or more intracellular signalling domains. The extracellular binding domain may comprise a single-chain variable fragment (scFv) from a monoclonal antibody. The extracellular binding domain may be specific for a tumour antigen. A first generation CAR typically comprises the CD37 chain domain or a modified derivative thereof as the intracellular signalling domain, which is the primary transmitter of signals.

In addition to the components specified for first generation CARs, second generation CARS also contain a co-stimulatory domain, such as CD28 and/or 4-1BB. The inclusion of an intracellular co-stimulatory domain improves T-cell proliferation, cytokine secretion, resistance to apoptosis, and in vivo persistence. The co-stimulatory domain of a second generation CAR is typically in cis with and upstream of the one or more intracellular signalling domains.

Third-generation CARs combine multiple co-stimulatory domains in cis with one or more intracellular signalling domains, to augment T-cell activity. For example, a third-generation CAR may comprise co-stimulatory domains derived from CD28 and 41BB, together with an intracellular signalling domain derived from CD3z. Other third-generation CARs may comprise co-stimulatory domains derived from CD28 and OX40, together with an intracellular signalling domain derived from CD3z.

Fourth-generation CARs (also known as TRUCKs or armoured CARs), combine the features of a second-generation CAR with further factors to enhance anti-tumour activity (e.g., cytokines, co-stimulatory ligands, chemokines receptors or further chimeric receptors of immune regulatory or cytokine receptors). The factors may be in trans or in cis with the CAR, typically in trans with the CAR.

In some embodiments, the CAR or TCR is specific for a cancer antigen. The cancer antigen may be a solid tumour cancer antigen. By “specific”, this will be understood to refer to being capable of specifically binding to a target antigen.

Cancer antigens include, but are not necessarily limited to Erbb1, Erbb3, Erbb4, Erbb2, mucins, PSMA, carcinoembryonic antigen (CEA), mesothelin, GD2, MUC1, folate receptor, GPC3, CAIX, FAP, NY-ESO-1, gp100, PSCA, ROR1, PD-L1, PD-L2, EpCAM, EGFRVIII, CD19, GD3, CLL-1, ductal epithelial mucin, CA-125, GP36, TAG-72, glycosphingolipids, glioma-associated antigen, beta-hCG, AFP (alpha-fetoprotein) and lectin-reactive AFP, thyroglobulin, receptor for advanced glycation end products (RAGE), TERT, telomerase, carboxylesterase, M-CSF, PSA, tyrosinase, survivin, PCTA-1, melanoma-associated antigen (MAGE), for example MAGE A1, MAGE A2, MAGE A4, MAGE A8, CD22, IGF-1, IGF-2, IGF-1 receptor, MHC-associated tumour peptide, 5T4, tumour stroma-associated antigens, WT1, MLANA, CA 19-9, epithelial tumour antigen (ETA), BCMA, cancer testis antigens such as CTA New York esophageal squamous cell carcinoma (NYESO) and glycoprotein 100 (GP100), preferentially expressed antigen in melanoma (PRAME), collagen type IV alpha 3 chain (COL6A3), MR1, CD1c, human epidermal growth factor receptor 2 (HER2), solute carrier family 3 member 2 (SLC3A2) and avb6 integrin.

In some embodiments, the cancer antigen is selected from NYESO, GP100, PRAME, COL6A3, MR1, CD1c, HER2, SLCA2, CD19, PSMA, AFP, CEA, CA-125, MUC1, ETA, tyrosinase and MAGE. In some embodiments, the CAR or TCR is an anti-CD19, anti-SLC3A2 or anti-PSMA CAR or TCR.

In some embodiments, the CAR or TCR is an anti-CD19, anti-PSMA, anti-HER2 or anti-GP100 CAR or TCR. In some embodiments, the CAR or TCR is an anti-HER2 or anti-GP100 CAR or TCR. In some embodiments, the CAR or TCR is an anti-CD19 or anti-PSMA CAR or TCR. In some embodiments, the CAR or TCR is an anti-PSMA CAR or TCR.

In some embodiments, the CAR is an anti-CD19, anti-PSMA or anti-HER2 CAR. In some embodiments, the CAR is an anti-PSMA or anti-HER2 CAR.

In some embodiments, the TCR is an anti-GP100 TCR.

MAGE may be selected from MAGE A1, MAGE A2, MAGE A4 or MAGE A8.

The CAR or TCR may be linked to a reporter protein.

The immune cell may comprise or consist of a primary cell. By “primary cell” this will be understood to refer to a cell that has been obtained from a subject. Primary cells are not immortalised cells from a cell line. The immune cell may comprise or consist of a primary T-cell. Optionally, the primary cell is a primary human T-cell.

The primary cell may be autologous. Alternatively, the primary cell may be allogeneic. In embodiments comprising a population of primary cells, the population may comprise a mixture of autologous and allogenic cells.

As the skilled person will appreciate, autologous cells are cells from the same subject, i.e. cells which have been obtained from a subject which will be administered back to the same subject. Allogeneic cells are cells obtained from a different subject to the subject to which the cells will be administered. The different subjects are typically from the same species. Allogenic cells are thus genetically different to the subject to which they are administered.

Alternatively, the immune cell may comprise or consist of an immortalised immune cell from a cell line.

Preferably, the exogenous ACS metabolism gene is undetectable in other cells, such as cancer cells. As such, other cells may be less able or unable to metabolise the ACS.

In some embodiments, the exogenous ACS metabolism gene is undetectable in cancer cells, inflammatory autoimmune cells and/or cancer associated cells. Preferably, the exogenous ACS metabolism gene is undetectable in cancer cells and/or cancer associated cells.

In some embodiments, the ACS cannot or is less able to be metabolised by other cells such as cancer cells, inflammatory autoimmune cells and/or cancer associated cells.

Preferably, the ACS cannot or is less able to be metabolised by cancer cells and/or cancer associated cells. For example, cancer cells, inflammatory autoimmune cells and/or cancer associated cells may have a normalised read count (of the exogenous ACS metabolism gene) of less than about 5, less than about 4, less than about 3, less than about 2, less than about 1, less than about 0.5, less than about 0.1 or less than about 0.01. Cancer cells, inflammatory autoimmune cells and/or cancer associated cells may have a normalised read count less than about 0.1 or less than about 0.01.

In some embodiments cancer cells, inflammatory autoimmune cells and/or cancer associated cells may have a log value (of the exogenous ACS metabolism gene) of less than less than about 1.2, less than about 1.1, less than about 1, less than about 0.9, less than about 0.8, less than about 0.7, less than about 0.6, less than about 0.5, less than about 0.4, less than about 0.3, less than about 0.2, less than about 0.1, less than about 0.09, less than about 0.09, less than about 0.07, less than about 0.06, less than about 0.05, less than about 0.04, less than about 0.03, less than about 0.02, less than about 0.01, less than about 0.009, less than about 0.008, less than about 0.007, less than about 0.006, less than about 0.005, less than about 0.004, less than about 0.003, less than about 0.002 or less than about 0.001. Cancer cell, inflammatory autoimmune cells and/or cancer associated cells may have a log value of less than about 0.02, less than about 0.01, less than about 0.009, less than about 0.008, less than about 0.007, less than about 0.006, less than about 0.005, less than about 0.004, less than about 0.003, less than about 0.002 or less than about 0.001.

Advantageously, in a pathogenic environment such as a tumour or an inflammatory lesion, the genetically engineered immune cells of the invention can utilise the ACS with less competition from the surrounding cells, especially cancer and/or cancer-associated cells. In the context of the present invention, cancer-associated cells will be understood to refer to regulatory T cells (tregs), myeloid-derived suppressor cells (MDSC), tumour-associated macrophages (TAMs) and cancer-associated fibroblasts (CAFs) within or adjacent to the tumour microenvironment.

In some embodiments, the immune cell is a T-cell, which is genetically engineered to express a chimeric antigen receptor and an exogenous trehalose metabolism gene which encodes the enzyme trehalase.

In some embodiments, the immune cell is a T-cell, which is genetically engineered to express a chimeric antigen receptor and an exogenous fructose metabolism gene which encodes the transporter GLUT5.

In other embodiments, the immune cell is a T-cell, which is genetically engineered to express a chimeric antigen receptor and an exogenous adenosine metabolism gene which encodes the enzyme ADA or ADA2.

In some embodiments, the immune cell is a T-cell, which is genetically engineered to express an anti-PSMA chimeric antigen receptor and an exogenous trehalose metabolism gene which encodes the enzyme trehalase. The anti-PSMA chimeric antigen receptor and enzyme trehalase may be encoded by a polynucleotide having SEQ ID NO: 13.

In some embodiments, the immune cell is a T-cell, which is genetically engineered to express an anti-PSMA chimeric antigen receptor and an exogenous fructose metabolism gene which encodes the transporter GLUT5. The anti-PSMA chimeric antigen receptor and transporter GLUT5 may be encoded by a polynucleotide having SEQ ID NO: 15.

In other embodiments, the immune cell is a T-cell, which is genetically engineered to express an anti-PSMA chimeric antigen receptor and an exogenous adenosine metabolism gene which encodes the enzyme ADA or ADA2, preferably ADA2. The anti-PSMA chimeric antigen receptor and enzyme ADA2 may be encoded by a polynucleotide having SEQ ID NO: 17.

In some embodiments, the immune cell is a T-cell, which is genetically engineered to express an anti-HER2 chimeric antigen receptor and an exogenous fructose metabolism gene which encodes the transporter GLUT5. The anti-HER2 chimeric antigen receptor and GLUT5 may be encoded by a polynucleotide having SEQ ID NO: 23.

In some embodiments, the immune cell is a T-cell, which is genetically engineered to express an anti-GP100 T cell receptor and an exogenous fructose metabolism gene which encodes the transporter GLUT5. The anti-GP100 T cell receptor and GLUT5 may be encoded by a polynucleotide having SEQ ID NO: 24.

Also provided is a cell which is genetically engineered to express an exogenous alternative carbon source (ACS) metabolism gene, wherein the ACS is not glucose and wherein the ability of the cell to metabolise the ACS is increased due to expression of the exogenous ACS metabolism gene. Any embodiments defined for the genetically engineered immune cell above, especially the CAR, TCR, ACS metabolism gene, expression, detectable amount, increased metabolism of the ACS and increased expression of the ACS metabolism gene equally apply to the cell. The cell may be, for example, a stem cell. The stem cell may be selected from an embryonic stem cell (ESC), tissue specific progenitor stem cell (TSPSC), mesenchymal stem cell (MSC), umbilical cord stem cell (UCSC), bone marrow stem cell (BMSC) and induced pluripotent stem cell (iPSC). In some embodiments the cell comprises an osteoblast, osteocytes, osteoclast, bone lining cell, neuron, myocyte, mesoangioblast, vascular smooth muscle cell, hepatocyte, β-cell, chondrocyte, retinal ganglion cell, cone cell or combination thereof. Such cells may have particular utility in regenerative medicine applications, for example implants or tissue replacement. By enabling metabolism of an alternative carbon source, the cells may have improved function and proliferation once transplanted, even in low glucose environments.

Suitable cells and regenerative medicine applications are provided in Table 1 below.

TABLE 1
Exemplary cells, diseases and applications for regenerative treatment.

| (a) Cell | (b) Derivation | (c) Disease/Condition |
|---|---|---|
| Embryonic stem cells (ESCs) | ESCs | Spinal cord injury |
| Retinal ganglion cells | ESCs | Glaucoma |
| Cone cells | ESCs | Age-related macular |
| | | degeneration (ARMD) |
| Cardiomyocytes | ESCs | Cardiovascular disease |
| Hepatocytes | ESCs | Liver injury |
| Pancreatic progenitor cells | ESCs | Diabetes |
| (PPCs)and/or β-cells | | |
| Chondrocytes | ESCs | Osteoarthritis |
| Pacemaker cells | ESCs | Cardiac arrhythmia |
| PPCs and/or β-cells | Tissue specific progenitor | Diabetes |
| | stem cells (TSPSCs) | |
| Dental pulp stem cells (DPSCs) | TSPSCs | Neurodental diseases |
| Inner ear stem cells and/or | TSPSCs | Hearing loss |
| auditory hair cells | | |
| Intestinal progenitor cells | TSPSCs | Intestinal degeneration |
| (IPCs) | | |
| Limbal progenitor stem cells | TSPSCs | Corneal disease |
| (LPSCs) | | |
| Mesoangioblasts (MABs) | TSPSCs | Muscular deformities |
| Adipose-derived stem cells | TSPSCs | Eye disease and/or diabetic |
| (AdSCs) | | retinopathy |
| Epithelial stem cells | TSPSCs | Fallopian tube degeneration |
| Vascular smooth muscle cells | TSPSCs | Wounds and/or scars |
| (VSMCs) | | |
| Cardiac adipose-derived stem | TSPSCs | Cardiovascular disease |
| cells (CA-AdSCs) and/or | | |
| mesangioblasts | | |
| Bone-marrow-derived | Mesenchymal stem cells | Bladder disease |
| mesenchymal stem cells | (MSCs) | |
| (BD-MSCs) | | |
| MSCs | MSCs | Muscle degeneration |
| EMSCs and DSCs | MSCs | Dental disease |
| MSCs | MSCs | Bone degeneration |
| Dermal papilla cells (DPCs) | MSCs | Alopecia |
| Macrophages | MSCs | Leishmania disease |
| MSCs | MSCs | Orthopaedic injury |
| MSCs | MSCs | Acute liver failure |
| MSCs | MSCs | Wounds and/or scars |
| Wharton's jelly-derived | Umbilical cord stem cells | Diabetes |
| mesenchymal stem cells | (UCSCs) | |
| (WJ-SCs) | | |
| Amniotic fluid stem cells | UCSCs | Congenital heart defects |
| (AFSCs) | | |
| WJ-SCs | UCSCs | Systemic lupus erythematosus |
| | | (SLE) |
| UCSCs | UCSCs | Lysosomal storage disease |
| | | (LSD) |
| UCSCs | UCSCs | Neurodegenerative disease |
| UCSCs | UCSCs | Hodgkin's lymphoma |
| UCSCs | UCSCs | Neuroblastoma |
| Umbilical cord blood stem | UCSCs | Cartilage and tendon injury |
| cells (UCB-SCs) | | |
| UCSCs | UCSCs | Krabbe's disease |
| WJ-SCs | UCSCs | Peritoneal fibrosis |
| Lymphoid and/or | Bone marrow stem cells | Anaemia |
| myeloid cells | (BMSCs) | |
| Lymphoid and/or | BMSCs | Blood cancer |
| myeloid cells | | |
| CD4⁺ hematopoietic | BMSCs | AIDS |
| (HSCs)and/or CD4⁺ progenitor | | |
| stem cells (PSCs) | | |
| BMSCs | BMSCs | Neurodegenerative disease |
| BMSCs | BMSCs | Orodental deformities |
| Megakaryocytes | BMSCs | Blood clotting disease |
| BMSCs | BMSCs | Traumatic brain injury |
| Bone-marrow derived | BMSCs | Diaphragm degeneration |
| mesenchymal stem cells | | |
| (BM-MSCs) | | |
| Induced pluripotent stem | Induced pluripotent | Kidney degeneration |
| cells (iPSCs) | stem cells (iPSCs) | |
| Neural progenitor cells (NPCs) | iPSCs | ARMD |
| Photoreceptor cells | iPSCs | Eye disease |
| Induced GABAergic | iPSCs | Neurodegenerative disease |
| interneurons (iGABA-Ins) | | |
| iPSCs | iPSCs | Autism spectrum disorder |
| | | (ASD) |
| iPSCs | iPSCs | Liver disease |
| iPSCs | IPSCs | Lung disease |
| iPSCs | iPSCs | SIDs and AIDs |
| Pacemaker cells | iPSCs | Cardiac arrhythmia |
| β-cells | IPSCs | Diabetes |
| Melanocytes | IPSCs | Skin disease |
| Heart valve cells | iPSCs | Heart valve degeneration |

The cell may comprise any of the cells specified in column (a) of Table 1 and is preferably derived from a cell specified in column (b) of Table 1. Further preferably, the cell may be used in a method of treating a disease in a subject, wherein the disease/condition is specified in column (c) of Table 1. More preferably, the cell comprises a cell specified in column (a) of Table 1, has the derivation specified in the same row in column (b) of Table 1 and is used to treat the disease/condition specified in the same row in column (c) of Table 1. For example, the cell may comprise a retinal ganglion cell, derived from an embryonic stem cell and may be used in a method of treating glaucoma in a subject.

Further provided is an implant comprising the cell/immune cell as defined above. The implant may comprise a hip replacement, knee replacement, cardiac implant, dental implant and/or replacement cartilage. The implant may comprise metal, ceramic, cartilage and/or plastic.

The invention also provides an isolated polynucleotide comprising an alternative carbon source (ACS) metabolism gene as defined above and a polynucleotide encoding a chimeric antigen receptor (CAR) and/or TCR as defined above. Any embodiments defined in the genetically engineered immune cell above, especially the CAR, TCR, and ACS metabolism gene, equally apply to the isolated polynucleotide. For example, the ACS metabolism gene may encode an enzyme. In some embodiments, the enzyme is selected from ADA, ADA2, LCT, MGAM, SI, Ketohexokinase (KHK), Aldolase B (ALDOB) and trehalase. Optionally, the enzyme is selected from Ketohexokinase (KHK), Aldolase B (ALDOB) and trehalase.

It will be appreciated that the polynucleotide is recombinant.

In some embodiments the isolated polynucleotide comprises from 5′ to 3′ the ACS metabolism gene and the polynucleotide encoding the exogenous receptor or ligand, optionally the CAR and/or TCR. The isolated polynucleotide may further comprise a polynucleotide encoding a ribosomal skipping sequence between the ACS metabolism gene and the polynucleotide encoding the CAR and/or TCR. The inclusion of a ribosomal skipping sequence enables the transcription and translation of the ACS metabolism gene and the CAR and/or TCR from only one isolated polynucleotide. Advantageously, this reduces the number of steps and time necessary to genetically engineer an immune cell using the isolated polynucleotide.

The isolated polynucleotide may further comprise a polynucleotide encoding a reporter gene. Suitable reporter genes include, but are not necessarily limited to HNIS, hNET and HSVtK.

A polynucleotide, such as a nucleic acid, is a polymer comprising two or more nucleotides. The nucleotides can be naturally occurring or artificial.

A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar and the nucleobase together form a nucleoside. Preferred nucleosides include, but are not limited to, adenosine, guanosine, 5-methyluridine, uridine, cytidine, deoxyadenosine, deoxyguanosine, thymidine, deoxyuridine and deoxycytidine. The nucleosides may be adenosine, guanosine, uridine and cytidine.

The nucleotides are typically ribonucleotides or deoxyribonucleotides. The nucleotides may be deoxyribonucleotides. The nucleotides typically contain a monophosphate, diphosphate or triphosphate. Phosphates may be attached on the 5′ or 3′ side of a nucleotide. Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (CAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides may be selected from AMP, UMP, GMP, CMP, dAMP, dTMP, dGMP or dCMP. In some embodiments, the nucleotides are selected from dAMP, dTMP, dGMP or dCMP.

The nucleotides may contain additional modifications. In particular, suitable modified nucleotides include, but are not limited to, 2′ amino pyrimidines (such as 2′-amino cytidine and 2′-amino uridine), 2′-hydroxyl purines (such as, 2′-fluoro pyrimidines (such as 2′-fluorocytidine and 2′ fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the isolated polynucleotide may be modified, for instance with a label or a tag. The label may be any suitable label which allows the nucleotides to be detected. Suitable labels include, but are not limited to, fluorescent molecules, radioisotopes, e.g. ¹²⁵I, ³⁵S, enzymes, antibodies, antigens, other polynucleotides and ligands such as biotin.

The nucleotides in the isolated polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups. The nucleotides may be connected via their nucleobases as in pyrimidine dimers.

The isolated polynucleotide may comprise a deoxyribonucleic acid (DNA) or a ribonucleic acid (RNA). The isolated polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains.

Unless otherwise indicated, a particular polynucleotide sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with a different base, mixed-base and/or deoxyinosine residues (Batzer et al., 1991, Nucleic Acid Res. 19:5081; Ohtsuka et al., 1985, J. Biol. Chem. 260:2605-2608; and Rossolini et al., 1994, Mol. Cell. Probes 8:91-98).

Substitutions may be used for the practices of codon optimisation and codon wobble, both of which are known to those skilled in the art. Thus, it will be appreciated that codon-optimised and codon-wobbled isolated polynucleotides are also envisaged. In an embodiment, the isolated polynucleotide is codon-optimised for human expression.

The isolated polynucleotides can be produced by de novo solid-phase DNA synthesis or by PCR mutagenesis of an existing sequence. Direct chemical synthesis of polynucleotides can be accomplished by methods known in the art, such as the phosphotriester method of Narang et al., 1979, Meth. Enzymol. 68:90; the phosphodiester method of Brown et al., 1979, Meth. Enzymol. 68:109; the diethylphosphoramidite method of Beaucage et al., 1981, Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., 1991, Nucleic Acids Res. 19:967; and Eckert et al., 1991, PCR Methods and Applications 1:17.

The invention also provides a vector comprising the isolated polynucleotide as defined above. In some embodiments, the vector is one vector. One-vector approaches advantageously introduce the ACS metabolism gene and the polynucleotide encoding the CAR and/or TCR, and any other optional polynucleotides, into the intended host cell at the same time. This reduces the time required to genetically engineer the cell, ensuring that the cell is kept in vitro for as short a time as possible. Without wishing to be bound by theory, the present inventors believe that this may further increase the efficacy of the genetically engineered immune cell.

In some embodiments, the vector is an expression vector. Various expression vectors can be employed to express the ACS metabolism gene and the CAR and/or TCR of the invention. Both viral-based and non-viral expression vectors can be used to produce the ACS metabolism gene and the CAR and/or TCR of the invention in a host cell, such as a mammalian host cell. Non-viral vectors and systems include plasmids, episomal vectors, typically with an expression cassette for expressing a protein or RNA, and human artificial chromosomes (see, e.g., Harrington et al., 1997, Nat Genet. 15:345). For example, non-viral vectors useful for expression of the ACS metabolism gene and the CAR and/or TCR of the invention in mammalian (e.g., human) cells include pThioHis A, B and C, pcDNA3.1/His, pEBVHis A, B and C, (Invitrogen, San Diego, Calif.), MPS V vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, 1995, Annu. Rev. Microbiol. 49:807; and Rosenfeld et al., 1992, Cell 68:143. In particular, retroviral, lentiviral, adenoviral or adeno-associated viral vectors are commonly used for expression in immune cells such as T-cells. Examples of such vectors include the SFG retroviral expression vector (see Riviere et al., 1995, Proc. Natl. Acad. Sci. (USA) 92:6733-6737).

In some embodiments, the vector is a retroviral or lentiviral vector. Optionally, the vector is an SFG retroviral vector. In some embodiments the vector is a lentiviral vector. Lentiviral vectors include self-inactivating lentiviral vectors (so-called SIN vectors).

The choice of expression vector depends on the intended host cells in which the vector is to be expressed. Expression vectors for mammalian host cells can include expression control sequences, such as an origin of replication, a promoter, and an enhancer (see, e.g., Queen, et al., 1986, Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPS V promoter, the tetracycline-inducible CMV promoter (such as the human immediate-early CMV promoter), the constitutive CMV promoter, the EF1 alpha promoter, the phosphoglycerate kinase (PGK) promoter and promoter-enhancer combinations known in the art.

Cultures of transformed organisms can be expanded under non-inducing conditions without biasing the population for coding sequences whose expression products are better tolerated by the host cells. In addition to promoters, other regulatory elements may also be required or desired for efficient expression of the ACS metabolism gene of the invention. These elements typically include an ATG initiation codon and adjacent ribosome binding site or other sequences. In addition, the efficiency of expression may be enhanced by the inclusion of enhancers appropriate to the cell system in use (see, e.g., Scharf et al., 1994, Results Probl. Cell Differ. 20:125; and Bittner et al., 1987, Meth. Enzymol., 153:516). For example, the SV40 enhancer or CMV enhancer may be used to increase expression in mammalian host cells.

The genetic engineering of immune cells can be carried out according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). The vector of the invention may be introduced into the immune cell using such techniques.

Also provided are host cells comprising the isolated polynucleotide and/or vector as defined above. The isolated polynucleotide and/or vector may be transfected into a host cell by standard techniques.

The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Alternatively, the isolated polynucleotide or vector may be delivered into the host cell by transduction. For example, a viral vector, as disclosed above, may be used for delivery of the isolated polynucleotide or vector.

It is possible to express the ACS metabolism gene and the CAR and/or TCR of the invention in either prokaryotic or eukaryotic host cells. Representative host cells include many E. coli strains, mammalian cell lines, such as CHO, CHO-K1, and HEK293; insect cells, such as Sf9 cells; and yeast cells, such as S. cerevisiae and P. pastoris.

In some embodiments the host cell is an immune cell as defined herein. Cell lines which may be used include the NK cell line NK-92.

Mammalian host cells for expressing the ACS metabolism gene and the CAR and/or TCR of the invention include Chinese Hamster Ovary (CHO cells) (including dhfr-CHO cells, described Urlaub and Chasin, 1980, Proc. Natl. Acad. Sci. USA 77:4216-4220 used with a DH FR selectable marker, e.g., as described in R. J. Kaufman and P. A. Sharp, 1982, Mol. Biol. 159:601-621), NSO myeloma cells, COS cells and SP2 cells. In some embodiments the host cells are CHO K1PD cells. In some embodiments the host cells are NSO1 cells. In particular, for use with NSO myeloma cells, another expression system is the GS gene expression system shown in WO 87/04462, WO 89/01036 and EP 338,841. When recombinant expression vectors encoding protein/polypeptide(s) are introduced into mammalian host cells, the protein encoded by the ACS metabolism gene and the CAR and/or TCR of the invention may be produced by culturing the host cells for a period of time sufficient to allow for expression of protein encoded by the ACS metabolism gene and the CAR and/or TCR in the host cells.

The invention further provides a method of genetically engineering an immune cell, the method comprising introducing an exogenous ACS metabolism gene as defined above into the immune cell, wherein the ACS is not glucose. Optionally, the method further comprises introducing a polynucleotide encoding an exogenous receptor or ligand, optionally an exogenous CAR and/or TCR, into the immune cell. Introduction may be simultaneous or sequential.

The exogenous ACS metabolism gene may be introduced into the immune cell as the isolated polynucleotide or the vector as defined above. In some embodiments, the method comprises transfecting the exogenous ACS metabolism gene into the immune cell. In other embodiments, the method comprises transducing the exogenous ACS metabolism gene into the immune cell. The method may further comprise culturing the immune cell such that the protein encoded by the ACS metabolism gene and the exogenous receptor or ligand, preferably a CAR and/or TCR, are expressed.

Various methods for the culture of immune cells are well known in the art. See, for example, Parente-Pereira A C et al. 2014, J. Biol. Methods 1 (2): e7, Ghassemi S et al. 2018, Cancer Immunol Res 6 (9): 1100-1109, and Denman C J et al. 2012, PLOS One 7 (1): e30264.

Also provided is a pharmaceutical composition comprising the genetically engineered cell/immune cell, the isolated polynucleotide and/or the vector as defined above and a pharmaceutically or physiologically acceptable diluent and/or carrier.

The carrier and/or diluent is generally selected to be suitable for the intended mode of administration and can include agents for modifying, maintaining, or preserving, for example, the pH, osmolarity, viscosity, clarity, colour, isotonicity, odour, sterility, stability, rate of dissolution or release, adsorption, or penetration of the composition. Typically, these carriers and/or diluents include aqueous or alcoholic/aqueous solutions, emulsions, or suspensions, including saline and/or buffered media.

Suitable further agents for inclusion in the pharmaceutical compositions include, but are not limited to, amino acids (such as glycine, glutamine, asparagine, arginine, or lysine), antimicrobials, antioxidants (such as ascorbic acid, sodium sulphite, or sodium hydrogen-sulphite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), colouring, flavouring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, excipients and/or pharmaceutical adjuvants.

The carrier and/or diluent may be a parenteral, optionally intravenous vehicle. Suitable parenteral vehicles include sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases, one might include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents, and inert gases, may also be present. The precise formulation will depend on the route of administration. Additional relevant principle, methods and components for pharmaceutical formulations are well known (see, e.g., Allen, Loyd V. Ed, (2012) Remington's Pharmaceutical Sciences, 22nd Edition).

A pharmaceutical composition of the present invention can be administered by one or more routes of administration using one or more of a variety of methods known in the art. As will be appreciated by the skilled person, the route and/or mode of administration will vary depending upon the desired results. Routes of administration for pharmaceutical compositions of the invention include intravenous, intramuscular, intradermal, intraperitoneal, intrapleural, subcutaneous, intratumoural, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoural, intrapleural and intra-sternal injection and infusion. In some embodiments, the pharmaceutical composition is administered intratumourally. In other embodiments, administration is intrapleural or intraperitoneal. When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

Alternatively, the pharmaceutical composition of the invention can be administered by a nonparenteral route, such as a topical, epidermal, or mucosal route of administration, for example, intranasally, orally, vaginally, rectally, sublingually, or topically.

In some embodiments, the pharmaceutical composition is for subcutaneous administration. Typically, the pharmaceutical compositions for subcutaneous administration contain suitable stabilizers (e.g., amino acids, such as methionine, and or saccharides such as sucrose), buffering agents and tonicifying agents. Alternatively, the pharmaceutical composition may be for intravenous administration.

The invention also provides a kit comprising the genetically engineered cell/immune cell, isolated polynucleotide and/or vector of the invention. The kit may further comprise instructions for use. In some embodiments, the genetically engineered cell/immune cell, isolated polynucleotide and/or vector is provided in an aqueous solution, optionally buffered solution and/or at a temperature of at least −20° C.

Also provided is a method of treating or preventing a disease in a subject, wherein the method comprises administering to the subject the genetically engineered cell, genetically engineered immune cell, isolated polynucleotide, vector and/or the pharmaceutical composition of the invention.

Regenerative applications using the genetically engineered cell are discussed in more detail above.

Preferably, the genetically engineered cell/immune cell is a population of genetically engineered cells/immune cells.

The method typically comprises administering a therapeutically effective amount or a prophylactically effective amount of the genetically engineered cell, genetically engineered immune cell or the pharmaceutical composition of the invention. A therapeutically effective amount is an amount which ameliorates one or more symptoms, such as all the symptoms, of the disease and/or abolishes one or more symptoms, such as all the symptoms, of the disease. The therapeutically effective amount preferably cures the disease. A prophylactically effective amount is an amount which prevents the onset of the disease and/or prevents the onset of one or more symptoms, such as all the symptoms, of the disease. The prophylactically effective amount preferably prevents the subject from developing the disease. Suitable amounts are discussed in more detail below.

The genetically engineered cell, genetically engineered immune cell or the pharmaceutical composition of the invention may be administered to a subject that displays symptoms of disease. The genetically engineered cell, genetically engineered immune cell or the pharmaceutical composition of the invention may be administered to a subject that is asymptomatic, i.e. does not display symptoms of disease. The genetically engineered cell, genetically engineered immune cell or the pharmaceutical composition of the invention may be administered when the subject's disease status is unknown or the patient is expected not to have a disease. The genetically engineered cell, genetically engineered immune cell or the pharmaceutical composition of the invention may be administered to a subject that is predisposed, such as genetically predisposed, to developing the disease.

The subject may be a mammal. Optionally, the subject is a human, horse, dog or cat. In some embodiments, the subject is human. Alternatively, the subject may be a horse.

Various diseases are suitable for treatment or prophylaxis by administration of the genetically engineered immune cell or pharmaceutical composition of the invention. Any disease which can be treated or prevented using immunotherapy is envisaged.

In some embodiments, the disease may comprise an inflammatory disease, fatigue condition or cancer.

Examples of inflammatory diseases include, but are not necessarily limited to autoimmune disease, allergy, asthma, coeliac disease, nephritis, hepatitis, reperfusion injury, graft versus host disease (GvHD) and transplant rejection.

In embodiments where the disease comprises graft versus host disease or transplant rejection, the genetically engineered immune cell or the pharmaceutical composition of the invention may be administered to the subject prior to transplantation. In this way, the severity or development of graft versus host disease or transplant rejection can be reduced or avoided.

By “fatigue condition”, this will be understood to refer to a condition comprising extreme tiredness. Fatigue condition may be selected from chronic fatigue syndrome (CFS/ME), fibromyalgia and post-viral or bacterial fatigue syndrome.

In some embodiments, the disease is an inflammatory disease. The inflammatory disease may be selected from autoimmune disease, asthma, graft versus host disease (GvHD), transplant rejection and combinations thereof.

In some embodiments, the disease is selected from autoimmune disease, asthma, graft versus host disease, transplant rejection, cancer and combinations thereof.

The disease may be selected from autoimmune disease and cancer.

Autoimmune disease may comprise rheumatoid arthritis, system lupus erythematosus (lupus), inflammatory bowel disease, multiple sclerosis, diabetes, Guillain-Barre syndrome, chronic inflammatory demyelinating polyneuropathy, Graves' disease, Hashimoto's thyroiditis, Myasthenia gravis, Vasculitis or combinations thereof.

In some embodiments, the disease is selected from rheumatoid arthritis, lupus, multiple sclerosis, diabetes and cancer.

In some embodiments, the disease is selected from rheumatoid arthritis, lupus, multiple sclerosis and diabetes.

In embodiments where the disease is an inflammatory disease or a fatigue condition, the genetically engineered immune cells may be genetically engineered regulatory immune cells, such as genetically engineered Treg cells.

Optionally, the disease is cancer and the cell is a genetically engineered immune cell. Preferably, the genetically engineered immune cell is a population of genetically engineered lymphocytes, more preferably a population of genetically engineered T-cells and most preferably a population of genetically engineered primary human CAR T-cells.

The cancer may include, but not necessarily be limited to, a solid tumour cancer, a soft tissue tumour, a metastatic lesion, and a haematological cancer. For example, the cancer can be liver cancer, lung cancer, breast cancer, prostate cancer, lymphoid cancer, colon cancer, renal cancer, bone cancer, pancreatic cancer, skin cancer, cancer of the head or neck, such as squamous cell carcinoma of the head and neck (SCCHN), cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukaemias including acute myeloid leukaemia, chronic myeloid leukaemia, acute lymphoblastic leukaemia, chronic lymphocytic leukaemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, myelodysplastic syndrome (MDS), chronic myelogenous leukaemia-chronic phase (CMLCP), diffuse large B-cell lymphoma (DLBCL), cutaneous T-cell lymphoma (CTCL), peripheral T-cell lymphoma (PTCL), hepatocellular carcinoma (HCC), gastrointestinal stromal tumours (GIST), non-small cell lung carcinoma (NSCLC), cutaneous melanoma, mucosal melanoma, cutaneous squamous cell carcinoma (CSCC), small-cell lung cancer, squamous cell carcinoma of the lung, Merkle cell carcinoma, environmentally induced cancers including those induced by asbestos, and combinations of said cancers. In embodiments, the cancer is selected from the above group.

Preferably, the cancer is a solid tumour cancer.

In some embodiments, the cancer is selected from the group consisting of cancer of the head and/or neck, ovarian cancer, malignant mesothelioma, breast cancer, pancreatic cancer, colorectal cancer, lung cancer, gastric cancer, bladder cancer, prostate cancer, oesophageal cancer, endometrial cancer, hepatobiliary cancer, duodenal carcinoma, thyroid carcinoma, cancer of the central nervous system or renal cell carcinoma.

In some embodiments, the cancer is selected from ovarian cancer, breast cancer, optionally triple-negative breast cancer, pancreatic cancer, malignant mesothelioma, and combinations of said cancers.

The subject may have been pre-treated with a chemotherapeutic agent.

The administration of the genetically engineered immune cell or the pharmaceutical composition of the invention to the subject may result in a decrease in tumour size of about 10%, about 20%, about 30%, about 40%, about 50%, about 60%, about 70%, about 80%, about 90%, or even about 100%, when compared to an untreated tumour.

In some embodiments, the method further comprises administering to the subject the alternative carbon source (ACS). Administration of the ACS may be simultaneous, sequential or separate to the genetically engineered cell, genetically engineered immune cell or the pharmaceutical composition of the invention. In some embodiments, the ACS is a monosaccharide, nucleoside or disaccharide. The monosaccharide may comprise or consist of fructose. The nucleoside may comprise or consist of cytidine, uridine, guanosine, inosine, thymidine, adenosine or combinations thereof. Preferably, the nucleoside is selected from guanosine and adenosine.

In some embodiments, the ACS is fructose or adenosine.

In some embodiments, the ACS is fructose. Alternatively, the ACS may be adenosine.

In some embodiments, the disaccharide is selected from sucrose, lactose, maltose, isomaltose and trehalose.

In some embodiments, the disaccharide is trehalose. Trehalose may comprise or consist of αα-trehalose, αβ-trehalose, ββ-trehalose, or a combination thereof. In some embodiments, the disaccharide is αα-trehalose.

In some embodiments, the ACS is trehalose, fructose or adenosine. The ACS may be trehalose or adenosine. Preferably, the ACS is trehalose or fructose. More preferably, the ACS is trehalose.

In some embodiments, the method comprises:
i) administering to the subject the genetically engineered cell, genetically engineered immune cell, isolated polynucleotide, vector and/or the pharmaceutical composition of the invention; and
ii) administering to the subject the ACS, preferably wherein the ACS is trehalose or fructose.

The ACS may be administered before, simultaneously and/or after the administration of the genetically engineered cell, genetically engineered immune cell, isolated polynucleotide, vector and/or the pharmaceutical composition of the invention. Preferably, the ACS is administered after the administration of the genetically engineered cell, genetically engineered immune cell, isolated polynucleotide, vector and/or the pharmaceutical composition of the invention.

In some embodiments, the ACS is repeatedly administered to the subject. For example, the ACS may be administered to the subject monthly, weekly or daily. In some embodiments, the ACS is administered to the subject daily, preferably after administration of the genetically engineered cell, genetically engineered immune cell, isolated polynucleotide, vector and/or the pharmaceutical composition of the invention.

Any suitable form of administration may be used for the ACS. For example, the ACS may be administered subcutaneously, intranasally, orally, topically, intraperitoneally or intravenously. In some embodiments, the ACS may be intraperitoneally administered.

In embodiments wherein the genetically engineered cell/immune cell comprises a population of genetically engineered cells/immune cells, the number of genetically engineered cells/immune cells administered to the subject should take into account the route of administration, the cancer being treated, the weight of the subject and/or the age of the subject. In general, from about 1×10⁶ to about 1×10¹¹ genetically engineered cells/immune cells are administered to the subject. In some embodiments, from about 1×10⁷ to about 1×10¹⁰ genetically engineered cells/immune cells, or from about 1×10⁸ to about 1×10⁹ genetically engineered cells/immune cells are administered to the subject.

Further provided is a method of repairing or replacing cells in a subject, wherein the method comprises administering to the subject the genetically engineered cell, genetically engineered immune cell or the pharmaceutical composition of the invention. The method may be for repairing or replacing tissue in a subject. In some embodiments, an implant, as defined above, comprises the genetically engineered cell.

The invention also provides the genetically engineered cell, genetically engineered immune cell, isolated polypeptide, vector and/or pharmaceutical composition of the invention for use in any of the therapeutic methods described above. Thus, also provided is the genetically engineered cell, genetically engineered immune cell, isolated polynucleotide, vector and/or the pharmaceutical composition of the invention for use in the treatment or prevention of a disease. In particular, the invention provides the genetically engineered immune cell, isolated polynucleotide, vector and/or the pharmaceutical composition of the invention for use in the treatment or prevention of cancer.

Also provided is the use of the genetically engineered cell, genetically engineered immune cell, isolated polypeptide, vector and/or pharmaceutical composition of the invention for the manufacture of a medicament for the treatment or prevention of a disease. Optionally, the disease is cancer. Further provided is use of the genetically engineered immune cell, isolated polypeptide, vector and/or pharmaceutical composition of the invention for the treatment or prevention of cancer.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is a schematic demonstrating how glucose competition limits T cell effector functions in the solid tumour microenvironment (TME);

FIG. 2 shows that CAR T-cell effector functions are inhibited by hypoglycaemia (low glucose concentration). Human T-cells expressing an anti-PSMA CAR (P28z) or expressing a control non-signaling CAR (PTr) were cocultured with PSMA expressing PC3-PSMA cells for 48 hrs in decreasing concentrations of glucose. After coculture, (A) cytotoxicity of CAR T-cells to PC3-PSMA cells was determined by MTT. (B) The proliferation of CAR T-cells was determined using CountBright beads and flow cytometry. (C) IFN-γ production by CAR T-cells was determined by intracellular cytokine staining and flow cytometry. Data represents mean+/−SEM from three independent donors.

FIG. 3 is a schematic demonstrating embodiments of the invention;

FIG. 4 shows that CAR T-cells expressing the exogenous enzyme trehalase (TREH) can utilise the alternative carbon source trehalose for cytotoxicity. PBMCs transduced with the PSMA-targeting CAR P28z alone (P28z, B) or in combination with TREH (TREH_4P28z, C), or expressing a signaling control CAR (PTr, A) were cultured at various effector: target ratios with PC3-PSMA cells for 72 hrs in glucose-free media containing either 10 mM glucose, 10 mM trehalose or no glucose/trehalose. After 72 hrs, the viability of PC3-PSMA cells was determined. Data represents mean+/−SEM of three independent donors.

FIG. 5 shows that expression of the exogenous enzyme TREH enables the proliferation of CAR T-cells in trehalose-containing media. PBMCs transduced with the PSMA-targeting CAR P28z alone (P28z) or in combination with TREH (4P28z_TREH), or expressing a signaling control CAR (PTr) were stained with CellTrace FarRed and then cultured at various effector: target ratios with PC3-PSMA cells for 96 hrs in glucose-free media containing either 10 mM glucose, 10 mM trehalose or no glucose/trehalose. After coculture, CellTrace fluorescence was determined by flow cytometry. (A) Representative CellTrace FarRed fluorescence after coculture (B) Average CellTrace FarRed median fluorescence intensity (MFI) after coculture. (C) The proliferation of CAR T-cells cocultured with PC3-PSMA cells at a 1:2 effector to target ratio for 72 hours in media containing either 25 mM glucose, 25 mM trehalose or no glucose/trehalose was determined using CellTrace FarRed proliferation dye and flow cytometry. Data represents mean+/−SEM of three independent donors.

FIG. 6 shows that expression of the exogenous glucose transporter GLUT5 enables the use of fructose by CAR T-cells for cytotoxicity. PBMCs transduced with the PSMA-targeting CAR P28z alone (P28z, B) or in combination with GLUT5 (GLUT5_4P28z, C), or expressing a signaling control CAR (PTr, A) were cultured at various effector: target ratios with PC3-PSMA cells for 72 hrs in glucose-free media containing either 10 mM glucose, 10 mM fructose or no glucose/fructose. After 72 hrs, the viability of PC3-PSMA cells was determined. Data represents mean+/−SEM of three independent donors.

FIG. 7 shows that expression of the exogenous glucose transporter GLUT5 enables the use of fructose by CAR T-cells for proliferation. PBMCs transduced with the PSMA-targeting CAR P28z alone (P28z) or in combination with GLUT5 (GLUT5_4P28z), or expressing a signaling control CAR (PTr) were stained with CellTrace FarRed and then cultured at various effector: target ratios with PC3-PSMA cells for 96 hrs in glucose-free media containing either 10 mM glucose, 10 mM fructose or no glucose/fructose. After coculture, CellTrace fluorescence was determined by flow cytometry. (A) Representative CellTrace FarRed fluorescence after coculture (B) Average CellTrace FarRed median fluorescence intensity (MFI) after coculture. Data represents mean+/−SEM of three independent donors.

FIG. 8 is a schematic showing a further embodiment of the invention;

FIG. 9 is a schematic showing an ADA2 construct design. The coding sequence of adenosine deaminase 2 (ADA2) was cloned upstream and in frame with a FLAG epitope tag and the CD8 hinge and transmembrane domain (CD8TM). Downstream of this is encoded a P2A skipping peptide and the CAR P28z.

FIG. 10 shows that the ADA2 construct of FIG. 9 is expressed in PBMCs following transduction.

FIG. 11 shows RNA expression levels of TREH in human tumour and normal cells (all non-genetically engineered cells). TREH RNAseq data from tumour (red) and matched normal (green) tissues was extracted from The Cancer Genome Atals (TCGA) and Genotype-Tissue Expression (GTEx) databases using GEPIA2. TCGA tumour subtype acronyms are displayed in appendix xyz. Tumour types where TREH expression is statistically significantly lower than matched normal tissue are indicated by green text. Statistical significance was calculated by two way ANOVA with a q-value cutoff of 0.01.

FIG. 12 shows RNA expression levels of GLUT5 in human tumour and normal cells (all non-genetically engineered cells). GLUT5 RNAseq data from tumour (red) and matched normal (green) tissues was extracted from The Cancer Genome Atals (TCGA) and Genotype-Tissue Expression (GTEx) databases using GEPIA2. TCGA tumour subtype acronyms are displayed in appendix xyz. Tumour types where GLUT5 expression is statistically significantly lower than matched normal tissue are indicated by green text and in red text where it is expressed at significantly higher levels. Statistical significance was calculated by two way ANOVA with a q-value cutoff of 0.01.

FIG. 13 shows that expression of the exogenous glucose transporter GLUT5 in CAR T-cells enhances the efficacy of the CAR T-cells in an in vivo tumour model. (A) Schedule of experiment. (B) Change in tumour volume (pooled shown in top graph and individual animals shown in bottom graph) over time: vertical dotted line represents day of CAR T-cell injection. (C) Kaplan-Meier curve of survival. (D) Weight change over time.

FIG. 14 shows that expression of GLUT5 enhances in vitro efficacy in a HER-2 specific CAR model. (A) Schematic showing construct design for co-expression of HER-2 specific CAR (4H28z) and GLUT5. (B) Cytotoxicity of PBMCs transduced with the HER-2-targeting 4H28z CAR alone (4H28z), in combination with GLUT5 (GLUT5_4H28z), or expressing a signaling control CAR (4HTr) against MCF-7 HER-2 positive breast cancer cell monolayers in glucose-free media supplemented with either 10 mM glucose, 10 mM fructose or no glucose/fructose. After 72 hrs, the viability of the MCF-7 HER-2 positive breast cancer cells was determined. Data represents mean+/−SEM of three independent donors. (C) Graphs show expression of GLUT5 enables the use of fructose by HER-2-specific CAR T-cells for proliferation. PBMCs transduced with the HER-2-targeting 4H28z CAR alone (4H28z), in combination with GLUT5 (GLUT5_4H28z), or expressing a signaling control CAR (4HTr) were stained with CellTrace FarRed and then cultured with MCF-7 HER-2 positive breast cancer cells for 96 hrs in glucose-free media containing either 10 mM glucose, 10 mM fructose or no glucose/fructose. After coculture, CellTrace fluorescence was determined by flow cytometry.

FIG. 15 shows that expression of GLUT5 enhances in vitro efficacy in a GP-100 specific TCR expressing model. (A) Schematic showing construct design for co-expression of GP-100 specific TCR (4gp100) and GLUT5. (B) Cytotoxicity of PBMCs transduced with the GP-100 targeting TCR alone (4gp100), in combination with GLUT5 (GLUT5_4gp100), or expressing a signaling control TCR (4) against HELA-gp-100 expressing cervical cancer cell line monolayers in glucose-free media containing either 10 mM glucose, 10 mM fructose or no glucose/fructose. After 72 hrs, the viability of the HELA-gp-100 expressing cervical cancer cell line was determined. Data represents mean+/−SEM of three independent donors. (C) Graphs show expression of GLUT5 enables the use of fructose by gp-100 specific TCR-expressing cells for proliferation. PBMCs transduced with the gp-100 targeting TCR alone (4gp100), in combination with GLUT5 (GLUT5_4gp100), or expressing a signaling control TCR (4) were stained with CellTrace FarRed and then cultured with HELA-gp-100 expressing cervical cancer cells for 96 hrs in glucose-free media containing either 10 mM glucose, 10 mM fructose or no glucose/fructose. After coculture, CellTrace fluorescence was determined by flow cytometry.

### EXAMPLES

### Materials and Methods

### Cloning of Lentiviral Transfer Plasmids

All cloning of lentiviral transfer plasmids was conducted using NEBuilder HiFi DNA Assembly. The pUltra backbone was prepared for assembly by digestion at 37° C. for one hour with appropriate restriction enzymes. The digested backbone was then purified using E.Z.N.A Cycle Pure DNA Clean Up Kit (Omega Bio-Tek) as per manufacturer instructions.

Inserts were prepared for assembly by PCR using Q5 Hot Start High-Fidelity DNA Polymerase. PCR primers were designed to specifically amplify the insert sequence from the template DNA while also adding >20 base pairs (bp) of sequence homologous to the digested ends of the backbone at the 3′ and 5′ end of the PCR product. Standard PCR reaction mixtures are displayed in Table 2 and standard PCR cycling conditions are displayed in Table 3, annealing temperatures were determined using NEB Tm Calculator. PCR primers and DNA templates for all inserts are listed in Table 4. 5 μL of insert PCR products were visualised by agarose gel electrophoresis to confirm correct amplicon size. The remaining PCR reaction was incubated for 15 mins with 0.25 μL of FastDigest DpnI (ThermoFisher Scientific) to remove template DNA, and PCR products were then purified using E.Z.N.A Cycle Pure DNA Clean Up Kit (Omega Bio-Tek) as per manufacturer instructions. For cloning of shRNA or shRNAmir inserts, double-stranded (ds) DNA fragments were generated by mixing complementary single-stranded (ss) DNA oligonucleotides (Integrated DNA Technologies). Mixtures were heated to 90° C. and then cooled to 25° C. at a rate of −2° C./min.

The concentration of purified backbone and insert DNA fragments were quantified using a Nanodrop TM 1000 Spectrophotometer (ThermoScientific). 30 ng of backbone and a mass of insert equivalent to a 1:1 molar ratio with the backbone were mixed with an equal volume of 2×NEBuilder HiFi DNA Assembly Mix (NEB) and incubated for 1 hr at 50° C.

TABLE 2
Standard PCR Reaction Components

| Reagent | Concentration | | Volume (μL) |
|---|---|---|---|
| DNA template | 1 | ng/μL | 1 |
| Forward Primer | 10 | μM | 1.25 |
| Reverse Primer | 10 | μM | 1.25 |
| Q5 Reaction Buffer | 5× | | 5 |
| Q5 High GC Enhancer | 5× | | 5 |
| dNTP Mix | 10 | mM | 0.5 |
| | each nucleotide | | |
| Q5 Hot Start Polymerase | 2000 | units/mL | 0.25 |

| | | |
|---|---|---|
| Nuclease Free Water | n/a | 10.75 |

TABLE 3
Standard PCR Cycling Conditions

| Step | Description | Temperature (° C.) | Time (s) |
|---|---|---|---|
| 1 | Initial Denaturation | 98 | 30 |
| 2 | Denaturation | 98 | 15 |
| 3 | Primer Annealing | variable | 15 |
| 4 | Extension | 72 | 30 per Kb of |
| | | | product |
| 5 | Repeat steps 2-4 a further 34 times | | |
| 6 | Final Extension | 72 | 120 |
| 7 | Hold | 10 | Indefinite |

TABLE 4
PCR Inserts Used For Construction of Lentiviral Transfer Plasmids

| | | Template |
|---|---|---|
| Insert | Primers (5′-3′) | DNA |
| P28ζ | F: ggctgcaggtccgatccaccggtcgccaccatggctctcccagtgactg | SFG-P285 |
| | (SEQ ID NO: 1) | |
| | R: tcgagtcgacgactccggaacgaattctgattagcgagggggcagggc | |
| | (SEQ ID NO: 2) | |
| PTr | F: ggctgcaggtccgatccaccggtcgccaccatggctctcccagtgactg | SFG-PTr |
| | (SEQ ID NO: 3) | |
| | R: tcgagtcgacgactccggaacgaattctgattacttgctccgcacccag | |
| | aagatg (SEQ ID NO: 4) | |
| T2A | F: aagcgcagcggctccggcgagggccggggcagcctgctg | pUltra |
| | (SEQ ID NO: 5) | |
| | R: gcagtcactgggagagccatgggtccggggttctcttccacgtcg | |
| | (SEQ ID NO: 6) | |
| P2A | F: ggatctggagcaacaaacttctcact (SEQ ID NO: 7) | pUltra |
| | R: aagcagtagggcagtcactgggagagccataggcccgggattctcctc | |
| | (SEQ ID NO: 8) | |
| TREH | F: cgtgaaaactacccctaaaagctagcgccaccatgccagggaggacctg | gBlock |
| | (SEQ ID NO: 9) | (IDT) |
| | R: aagtttgttgctccagatccccatggcaggaggctgagcaggagg | |
| | (SEQ ID NO: 10) | |
| GLUT5 | F: ccctaaaagctagcgccaccatggagcaacaggatcagagcatgaagg | OHu16406 |
| | (SEQ ID NO: 11) | |
| | R: agtgagaagtttgttgctccagatccctgttccgaagtgacaggtggaa | |
| | gc (SEQ ID NO: 12) | |
| HER2 CAR | F: GGAAGAGAACCCCGGACCCATGGGCCCCGGCGTGC (SEQ ID No: | gBlock |
| | 19) | (IDT) |
| | R: cgacgactccggaacgaattctgaTTATCTGGGAGGCAGGGCCTGCATG | |
| | (SEQ ID NO: 20) | |
| Gp100_TCR | F: GGAAGAGAACCCCGGACCCatggccctgccagtgaccg (SEQ ID | gBlock |
| | NO: 21) | (IDT) |
| | R: cgacgactccggaacgaattctgatcagcctctgctgtccttccgc | |
| | (SEQ ID NO: 22) | |

### Bacterial Transformation

NEBuilder HiFi Assembly reactions were transformed by mixing the entire reaction with 25 μL of NEB 5-alpha competent E. coli (NEB) and incubating on ice for 30 mins. The mixture was then heat shocked by incubating at 40° C. for 30 sec, before being returned to the ice for 5 mins. 250 μL of SOC recovery medium (NEB) was gently added to bacteria cells, and they were then incubated in a shaker incubator at 37° C. for 1 hr at 220 RPM. Bacteria were then plated on LB agar plates containing ampicillin (100 μg/mL) and incubated for 18 hrs at 37° C. The resulting colonies were miniprepped and screened by analytical restriction digestion and sanger sequencing.

### Miniprep

Ampicillin resistant bacterial colonies were transferred to 15 mL of LB broth containing ampicillin (100 g/mL) and grown at 37° C. for 18 hr at 220 RPM. Bacteria were pelleted by centrifugation at 4000 g for 5 min. The supernatant was removed, and plasmid DNA was extracted with E.Z.N.A Plasmid DNA Mini Kit II (Omega Bio-Tek). Bacteria were first resuspended in 500 μL of buffer 1, before being lysed by the addition of 500 μL of buffer 2. The lysis was neutralised by the addition of 700 μL of buffer 3. The mixture was then centrifuged at 16000 g for 5 mins, and the supernatant containing the plasmid DNA was transferred to a silica spin column. DNA was centrifuged onto the column at 16000 g for 1 min and the supernatant was discarded. The column was washed twice with 500 μL of DNA wash buffer, before a final ‘dry’ centrifugation step to fully remove all buffer from the silica. Finally, plasmid DNA was eluted by the addition of 100 μL of water to the column and centrifugation at 16000 g for 2 mins. Eluted DNA was quantified using a Nanodrop TM 1000 Spectrophotometer (ThermoScientific) and stored at −20° C. until use.

### Analytical Restriction Digests & Sanger Sequencing

Correct construction of plasmids was first confirmed by analytical restriction digests. Restriction enzymes that would result in unique digestion patterns for the desired plasmid were selected, and 1 μg of miniprepped plasmid was incubated with these restriction enzymes at 37° C. for 1 hr. Digested plasmids were then separated by agarose gel electrophoresis and imaged using a UV transilluminator. Plasmid preparations that resulted in a digestion pattern consistent with the desired plasmid were then submitted for Sanger sequencing (Genewiz). Sanger sequences were compared against the reference plasmid maps using Snapgene (Insightful Science) to confirm the correct assembly of the insert into the plasmid.

### Maxiprep

For transfection, DNA was maxi-prepped using the E.Z.N.A Plasmid DNA Maxi Kit (Omega Bio-Tek). Ampicillin resistant bacterial colonies were transferred to 1 mL of LB broth containing ampicillin (100 μg/mL) and grown at 37° C. for 8 hr at 220 RPM. 250 μL of this culture was then transferred to 250 ml of LB broth containing ampicillin (100 μg/mL) and grown at 37° C. for 18 hr at 220 RPM. Bacteria were pelleted by centrifugation at 4000 g for 10 min. The supernatant was removed, and the pellet was resuspended in 10 ml of buffer 1. Bacteria were lysed by the addition of 10 ml of buffer 2 and the tube was inverted several times. After 5 min, the lysis was neutralised by the addition of 12 mL of buffer 3. The mixture was then centrifuged at 20000 g for 10 min. The resulting supernatant was transferred to a maxi spin column which was centrifuged at 4000 g for 2 min. The supernatant was discarded at the spin column was washed twice with 15 mL of DNA wash buffer. The column was dried after the final was by a ‘dry’ centrifugation at 4000 g for 10 mins. DNA was eluted from the spin column by the addition of 2 mL of nuclease-free water.

The column was incubated with the water at room temperature for 5 mins before being centrifuged at 4000 g for 5 mins. The eluted DNA was transferred to a 2 ml conical tube and stored at −20° C. until use. Eluted DNA was quantified using a Nanodrop TM 1000 Spectrophotometer (ThermoScientific).

### Cell Lines

HEK-293T, PC3 and PC3-PSMA cells were cultured in Dulbecco's Modified Eagle Medium (DMEM) (Gibco) supplemented 10% Foetal Bovine Serum (FBS) (Sigma). Jurkat cells were cultured in Roswell Park Memorial Institute (RPMI) 1640 (Gibco) supplemented with 10% FBS. In experiments where media containing different concentrations of glucose or alterative to glucose are used, PC3-PSMA cells were cultured in glucose-free DMEM supplemented with 10% FBS. All cell lines were cultured at 37° C. in 5% CO₂.

### Lentiviral Production in HEK-293T

1×10⁷ HEK-293T cells were plated in 150 mm dishes and allowed to adhere for 24 hrs. HEK-293T cells were then transfected with 25 μg of lentiviral transfer plasmid, 12.5 μg pVSV-G and 12.5 μg of pCRV-1. Transfection reactions were prepared by mixing plasmids with 150 μg of polyethylenimine (Sigma) in 1 mL of serum-free DMEM and incubating at room temperature for 25 mins before reactions were added dropwise to cells. 24 hrs after transfection, media was aspirated and replaced with 17.5 ml of fresh media. 48 hrs after transfection, virus-containing media was removed and transferred to a 50 ml conical tube which was stored at 4° C. 17.5 mL of fresh media was added to cells. 72 hrs after transfection, virus-containing media was again removed from cells and pooled with the virus-containing media removed from cells the previous day.

Lentivirus was then concentrated from virus-containing media by first centrifuging at 1500 g for 5 min to remove detached cells and debris. The supernatant containing lentivirus was transferred to a new 50 ml conical tube and centrifuged at 20000 g for 90 mins. The supernatant was aspirated and the virus-containing pellet was resuspended in 200 μL of phosphate-buffered saline (PBS). Concentrated lentivirus was then split into 50 μL aliquots in 2 mL screw-top tubes and stored at −80° C. until used.

### Isolation, Transduction and Expansion of Human PBMCs

Whole blood was obtained from healthy human donors (research ethics approval: 09/H0804/92). PBMCs were purified by carefully layering whole blood over 15 mL of Ficoll-Paque (GR Healthcare), before centrifugation at 800 g for 35 min with brake and accelerator set to minimum. PBMCs were then transferred to a clean 50 ml tube and washed twice in PBS, before being resuspended in an appropriate volume of media.

PBMCs were cultured in RPMI 1640 (Gibco) supplemented with 5% human AB serum (Sigma). PBMCs were counted and diluted to 1×10⁶ cells/mL and activated with 5 μg/mL Phytohemagglutinin-L (Sigma). 24 hrs after activation, cultures were supplemented with 100 U/mL recombinant human IL-2 (rIL-2) (R&D Systems) and 50 μL of concentrated lentivirus was then added to cells and mixed by gently pipetting. For untransduced cells, 50 μL of PBS was added in place of lentivirus.

48 hrs after transduction, PBMCs were transferred to 1 mL of PBS and centrifuged at 800 g for 5 mins. The supernatant was discarded and the PBMC pellet was resuspended in 500 μL of fresh media supplemented with 100 U/mL rIL-2. In instances where PBMCs were transduced with constructs containing the chimeric cytokine receptor 4αβ, cultures were supplemented with 3 ng/ml recombinant human IL-4 (rIL-4) instead of rIL-2. Cells were then expanded to sufficient numbers by diluting with fresh media and supplementation of cultures with 100 U/mL rIL-2 or 3 ng/ml rIL-4 every 2-3 days.

Coculture of PC3-PSMA Cell Lines with PBMCs

PC3, PC3-PSMA, HELA-gp-100 and MCF-7 HER-2 positive cells were plated in either a 96-well, 24-well or 6-well tissue culture plate at a density of 1×10⁵ cells/cm². PC3 or PC3-PSMA cells were allowed to adhere for 24 hrs. PBMC were then counted, washed in excess PBS to remove all cytokine and resuspended in fresh media. PBMC were then added to wells containing PC3 or PC3-PSMA cells at the indicated effector:target (E:T) ratio and incubated for the indicated time period.

In experiments where defined concentrations of glucose or other carbon sources were used, PC3-PSMA cells were first plated in standard media and allowed to adhere overnight. Before the addition of PBMC, media was aspirated from PC3-PSMA cells, wells were washed with excess PBS, and standard media was replaced with glucose-free media. PBMC were then counted, washed in excess media, resuspended in glucose-free media and added to PC3-PSMA cultures. Glucose, Trehalose or Fructose (all Sigma) dissolved in PBS was then added to cultures to achieve the indicated concentration of the carbon source.

### Cytotoxicity Assay

Cytotoxicity of CAR transduced cells toward PC3-PSMA cells, HELA-gp-100 expressing cervical cancer cells or MCF-7 HER-2 positive breast cancer cells was determined using 3-(4,5-dimethylthiazol-2-yl)-2,5-diphenyltetrazolium bromide (MTT) assay. After coculture of PBMC and PC3/PC3-PSMA/HELA-gp-100 expressing cervical cancer cells/MCF-7 HER-2 positive breast cancer cells, the remaining media was completely aspirated and replaced with fresh DMEM supplemented with 10% FBS and 500 μg/mL MTT (Sigma). Plates were then incubated for 90 min at 37° C. in 5% CO₂. All media was then aspirated and formazan crystals resulting from remaining viable PC3/PC3-PSMA/HELA-gp-100 expressing cervical cancer cells/MCF-7 HER-2 positive breast cancer cells were solubilised in Dimethyl sulfoxide (DMSO) (Sigma).

The absorbance of this solution at 590 nm was measured using a FLUOstar Omega plate reader. Cytotoxicity was determined relative to control wells where PC3, PC3-PSMA, HELA-gp-100 expressing cervical cancer MCF-7 HER-2 positive breast cancer cells had been cultured in the absence of any PBMCs (Tumour Only). For cultures containing defined concentrations of glucose or other carbon sources, cytotoxicity was determined relative to PC3-PSMA cells, HELA-gp-100 expressing cervical cancer cells or MCF-7 HER-2 positive breast cancer cells cultured in the same concentration of glucose or other carbon sources. The viability of target cell cultures was calculated using the formula:

$$\% Viability = ( \frac{Absorbance}{Absorbance of Tumour Only} ) \times 100$$

### Flow Cytometry

All samples for flow cytometry were acquired using either a BD LSRFortessa Flow Cytometer and BD FACSDiva software or a Beckman CytoFLEX and CytExpert software. Flow cytometry data were analysed using FlowJo software (BD).

Counting of Cells with CountBright Beads

For counting cells with CountBright beads, cells were first washed twice in 1 mL of staining buffer. If necessary, cells were then stained for cell-surface protein expression as described above. Cells were then resuspended in 275 μL of staining buffer and 25 μL of CountBright Beads (Invitrogen) were added to each sample. Samples were then by flow cytometry and the number of cells events and bead events tabulated. The cell concentration was determined using the following formula:

$$Cell Count ( \frac{cells}{μ L} ) = ( \frac{( Cell Count \times 25 )}{( Bead Count \times 275 )} ) \times Counting Bead Concentration ( \frac{beads}{μ L} )$$

Analysis of Proliferation with CellTrace Far Red

For analysing PBMC proliferation, PBMCs were counted, washed in excess PBS and resuspended to a density of 1×10⁶ cells/mL in PBS containing 1 μM CellTrace Far Red (Invitrogen). PBMC suspensions were then incubated at 37° C. for 20 mins. Cells were then washed in excess RPMI supplemented with 5% human serum, resuspended in an appropriate volume of media and either cocultured with PC3-PSMA cells or stimulated with PMA and Ionomycin. 72-96 hrs after stimulation, cells were washed twice in 1 mL of staining buffer. If necessary, cells were then stained for cell-surface protein expression as described above. Cells were then resuspended in 300 μL of staining buffer and Cell Trace Far Red fluorescent signal was determined by flow cytometry.

### Xenograft Model and Adoptive CAR T-Cell Therapy

All animal experiments were conducted in accordance with UK Home Office guidelines under the project license P23115EBF. NOD scid gamma (NSG) mice were maintained under specific-pathogen free conditions.

The pharmacokinetics of intraperitoneal (IP) fructose delivery was determined by injecting mice with 300 mg/kg fructose dissolved in PBS via IP injection. At specified time points post-IP injection, 20 μL blood samples were collected from mice tail veins. Serum samples were obtained by centrifuging blood samples at 2000 g for 10 mins and transferring serum to a 1.5 mL conical tube. Samples were stored at −20° C. until use.

For CAR efficacy studies, mice were subcutaneously inoculated with 2.5×10⁶ PC3-LN3-PSMA cells. 9 days after tumour engraftment, mice were intravenously injected with 1×10⁶ CAR⁺ T cells. After CAR T cell adoptive transfer, mice received daily intraperitoneal injections of fructose (300 mg/kg). Tumour growth was monitored every 2-3 days by calipers measurement. Tumour volume was calculated as length×width²×(n/6). Mice weight was monitored every 7 days. For survival analysis, mice were humanely culled when either (1) tumour measured >13.5 mm in any direction, (2) tumour ulcerated, (3)>15% weight loss or (4) mice exhibited poor mobility or piloerection.

### Example 1: Glucose Competition Limits T Cell Effector Functions

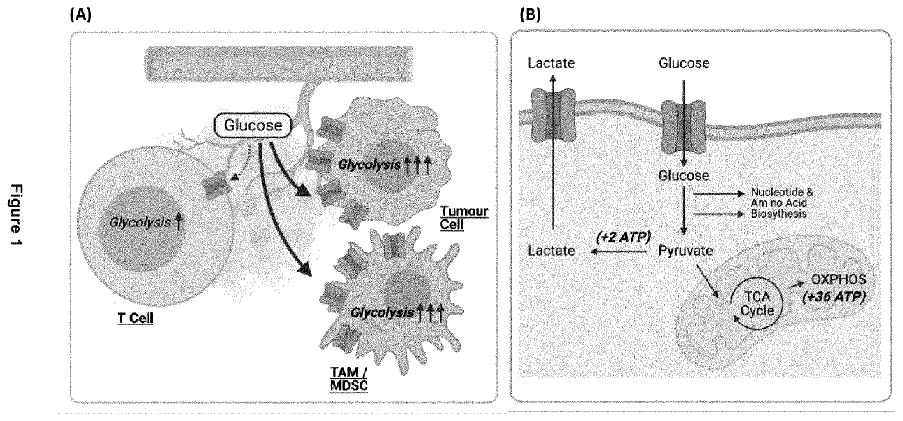

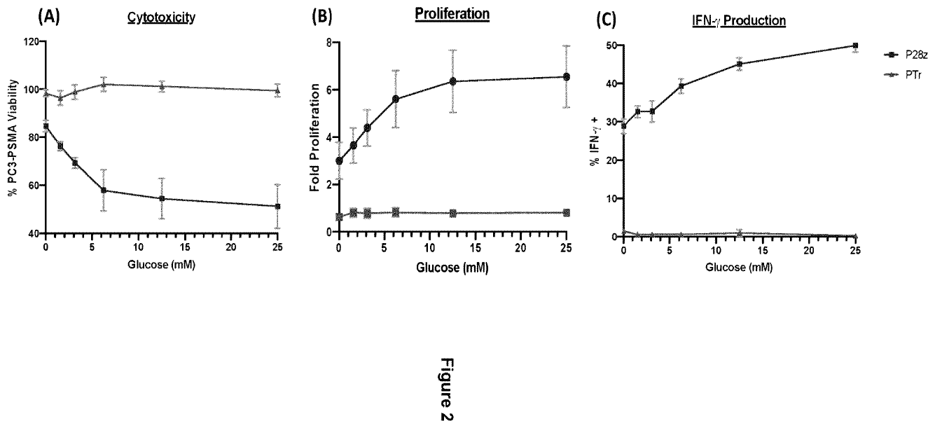

One immunosuppressive mechanism of the TME is competition between tumour cells and T cells for metabolites essential for cellular survival and proliferation. The best-characterised form of metabolic competition is for glucose. The solid TME is characterised by a poorly structured neo-vasculature and almost all tumours upregulate glucose transporters and glycolytic enzymes (FIG. 1A). Consequently, the extracellular glucose concentration in solid tumours is typically less than 1 mM. This is far lower than the extracellular glucose concentration of most organs (5 mM) and the standard cell culture media (25 mM) from which most in vitro data is obtained.

T-cells are highly reliant on glucose as a carbon source, and on anaerobic glycolysis for effector functions. T cells conduct anaerobic glycolysis even in the presence of oxygen levels sufficient to utilise oxidative phosphorylation (OXPHOS), a metabolic phenomenon termed the Warburg effect. Warburg metabolism is an almost ubiquitous feature of rapidly proliferating cells, including tumour cells. Although anaerobic glycolysis generates far less ATP per glucose molecule than OXPHOS, Warburg metabolism is favoured in quickly proliferating cells due to the more rapid kinetics of flux compared to OXPHOS, which requires the participation of mitochondrial pathways. This high flux allows cells to quickly recycle redox intermediates such as NAD⁺, and generate elevated levels of glycolytic intermediates for amino acid and nucleotide biosynthesis, sustaining rapid proliferation (FIG. 1B).

Because effector T-cells and tumour cells utilise the same metabolic pathway, anaerobic glycolysis, in an already glucose-limited environment, direct competition results between tumour cells and T-cells for glucose, with tumour cells outcompeting T-cells in established solid tumours.

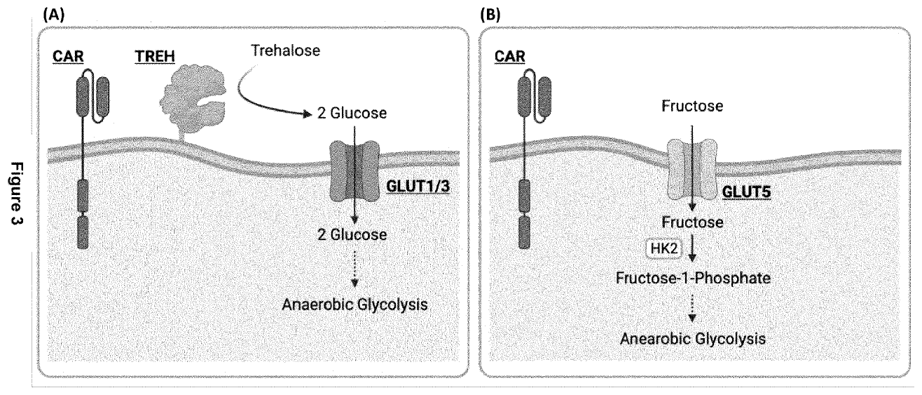

Our in vitro data (FIG. 2) confirms that the effector functions of CAR T-cells are severely inhibited by reduce glucose concentrations, mimicking those found in the solid TME. When co-cultured with PSMA-expressing cells in the presence of reducing glucose concentrations, P28z CAR T cells (which target the solid tumour antigen PSMA) are less cytotoxic, proliferate less and produce less IFN-γ (FIG. 2). Thus, metabolic competition for glucose is a key limiting factor in CAR T cell efficacy when targeting solid tumours.

### Example 2: CAR T Cells can be Engineered to Utilise the Alternative Carbon Sources Fructose and Trehalose

We have found that a CAR (or other such therapeutic molecule) can be co-expressed with an alternative carbon source (ACS) metabolism gene in an immune cell. This enables metabolism of the ACS in the immune cell, regardless of the extracellular glucose concentration. In addition, co-expression of a CAR or TCR with the ACS metabolism gene negates the need for any additional manipulation of CAR/TCR cells before adoptive transfer. This avoids additional complication or cost to manufacture. FIG. 3 is a schematic to show two of the alternative carbon source approaches we have developed.

Trehalose is a disaccharide composed of two glucose monomers that is impermeable to human cells. The human gene TREH encodes trehalase (TREH) which is expressed as a GPI-anchored extracellular enzyme. This protein is expressed predominantly in the luminal brush-border membrane of the small intestine, where its primary role is to catalyse the break-down of dietary trehalose to glucose. Since trehalose is impermeable to human cells, cells not expressing TREH (including immune cells) cannot utilise trehalose.

To enable immune cells to utilise trehalose as an alternative carbon source, CARs were co-expressed in T-cells with human trehalase (TREH) (FIG. 3A). This enabled T cells to metabolise the disaccharide trehalose to two glucose molecules, which can then be taken up by endogenous glucose transporters expressed in T cells, GLUT1 and GLUT3, and metabolized via anaerobic glycolysis. In subsequent experiments, the P28z CAR (which is specific for the solid tumour antigen PSMA) was used.

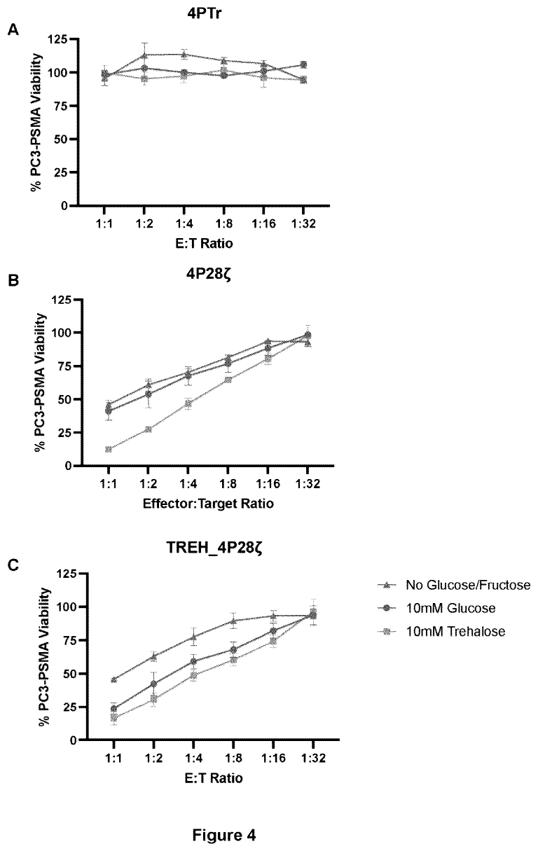

The cytotoxicity of exogenous trehalase expressing CAR T-cells was considered (FIG. 4). PBMCs transduced with the PSMA-targeting CAR P28z alone (P28z), in combination with TREH (TREH_4P28z), or expressing a signaling control CAR (PTr) were cultured at various effector: target ratios with PC3-PSMA cancer cells for 72 hrs in glucose-free media containing either 10 mM glucose, 10 mM trehalose or no glucose/trehalose. TREH_4P28z was encoded by the nucleotide sequence SEQ ID NO: 13, with the resulting amino acid sequence shown as SEQ ID NO: 14. After 72 hrs, the viability of PC3-PSMA cells was determined. Exogenous expression of TREH in anti-PSMA CAR T cells (TREH_P28z) rescued cytotoxicity towards the PSMA-expressing prostate cancer cells under hypoglycemic conditions in the presence of trehalose (FIG. 4C as compared to FIG. 4B).

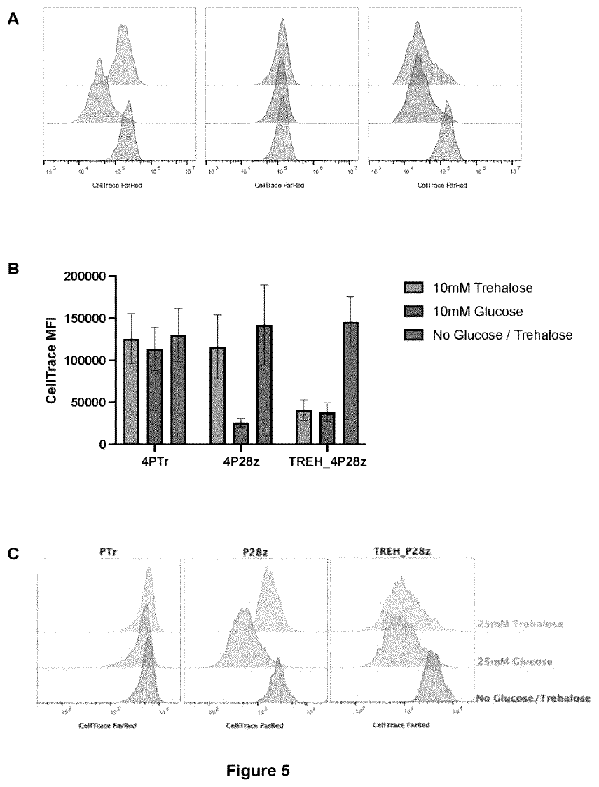

The proliferation of the CAR T-cells was then assessed (FIG. 5). PBMCs transduced with the PSMA-targeting CAR P28z alone (P28z), in combination with TREH (4P28z_TREH), or expressing a signaling control CAR (PTr) were stained with CellTrace FarRed and then cultured at various effector: target ratios with PC3-PSMA cells for 96 hrs in glucose-free media containing either 10 mM glucose, 10 mM trehalose or no glucose/trehalose. After coculture, CellTrace fluorescence was determined by flow cytometry. Control T-cells which expressed a signaling control CAR (PTr) had limited proliferation in all conditions. Although the CAR T-cells without trehalase (P28z) were able to proliferate in the presence of glucose, in the absence of glucose (either no glucose and trehalose, or trehalose only), proliferation did not occur. In contrast, trehalase-expressing CAR T-cells (TREH_P28z) proliferated in glucose and trehalose conditions (see FIGS. 5A and 5B). This effect was also observed at a concentration of 25 mM glucose or trehalose (FIG. 5C).

Another alternative carbon source is fructose. Fructose is a pentose sugar that constitutes the second most abundant monosaccharide in humans. The fortification of western diets with high-fructose corn syrup means that fructose is increasingly abundantly available in human circulation. Dietary fructose is transported into circulation via the expression of fructose transporters GLUT2, GLUT5 and GLUT8, of which GLUT5 is the only specific fructose transporter. Within the liver, the primary site of fructose metabolism, fructose is metabolised via ketohexokinase (KHK) and Aldolase B (ALDOB) to intermediates that can be utilised for glycolysis or gluconeogenesis. Some other tissues such as muscle and certain parts of the brain metabolise fructose.

Therefore, as a complementary approach, a CAR was co-expressed in T-cells with the transporter GLUT5 (FIG. 3B). This enabled transport of fructose across the cell membrane. Once inside the cell, fructose can be utilised as an ACS by phosphorylation to fructose-1-phosphate by ubiquitously expressed hexokinase (HK2). Fructose-1-phosphate can then be metabolised via anaerobic glycolysis.

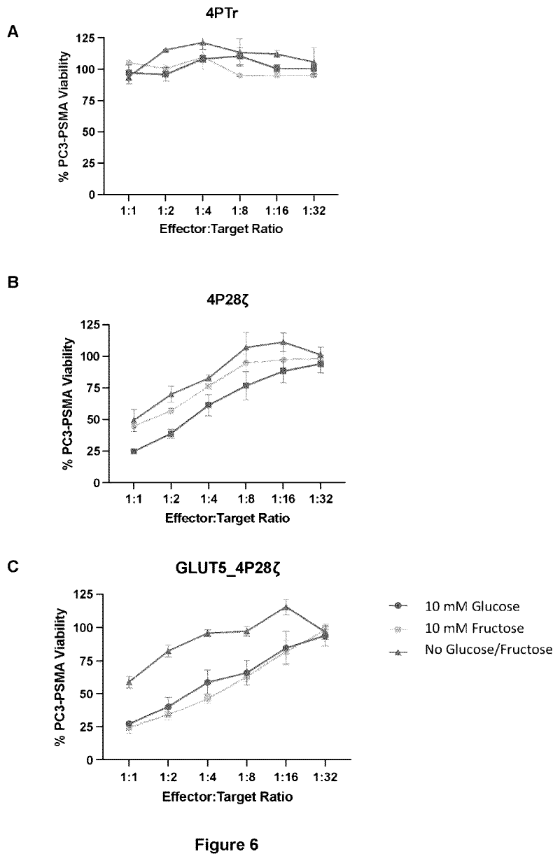

The effect of expression of exogenous GLUT5 on the cytotoxicity of the CAR T-cells is shown in FIG. 6. Exogenous expression of GLUT5 in anti-PSMA CAR T cells (GLUT5_P28z) rescued cytotoxicity towards the PSMA-expressing prostate cancer cells under hypoglycemic (no glucose) conditions in the presence of fructose (FIG. 6C as compared to FIG. 6B). GLUT5_4P28z was encoded by the nucleotide sequence SEQ ID NO: 15, with the resulting amino acid sequence being SEQ ID NO: 16.

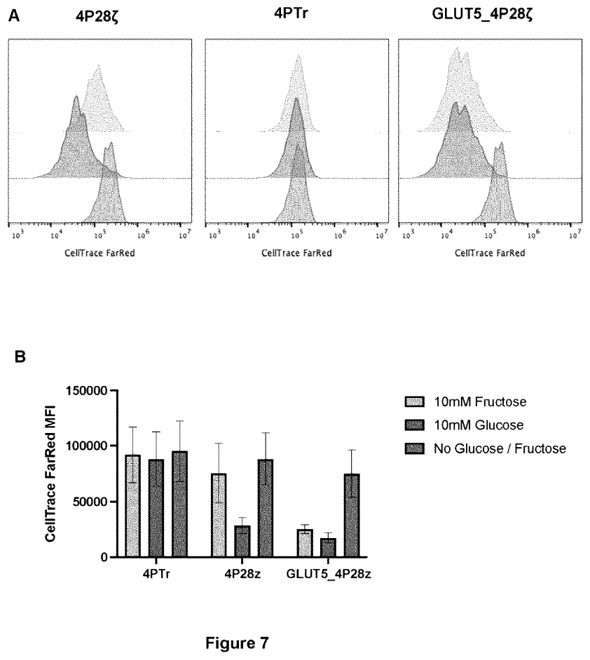

The exogenous expression of GLUT5 in the CAR T-cells also enabled these cells to proliferate in fructose-containing glucose-free media (FIG. 7). In contrast, CAR T-cells which did not express GLUT5 were not able to proliferate in fructose-containing glucose-free media. Proliferation in the presence of fructose only became comparable to proliferation in the presence of glucose (FIGS. 7A and B).

### Example 3: CAR T Cells can be Engineered to Utilise the Alternative Carbon Source Adenosine

Inosine has been demonstrated to act as a naturally occurring ACS for CAR T cells. We hypothesised that the expression of membrane bound adenosine deaminase 2 (ADA2TM) would enable conversion of adenosine to inosine in the TME.

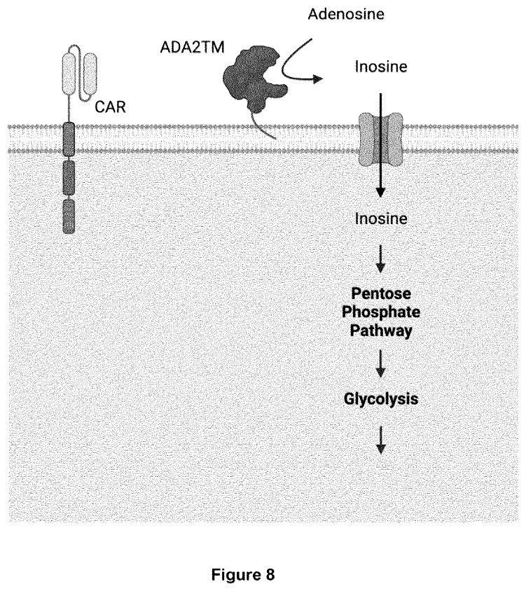

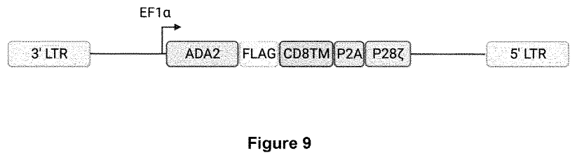

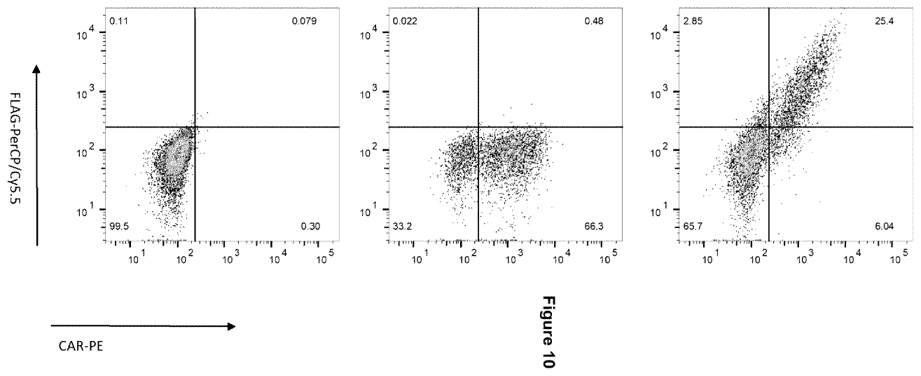

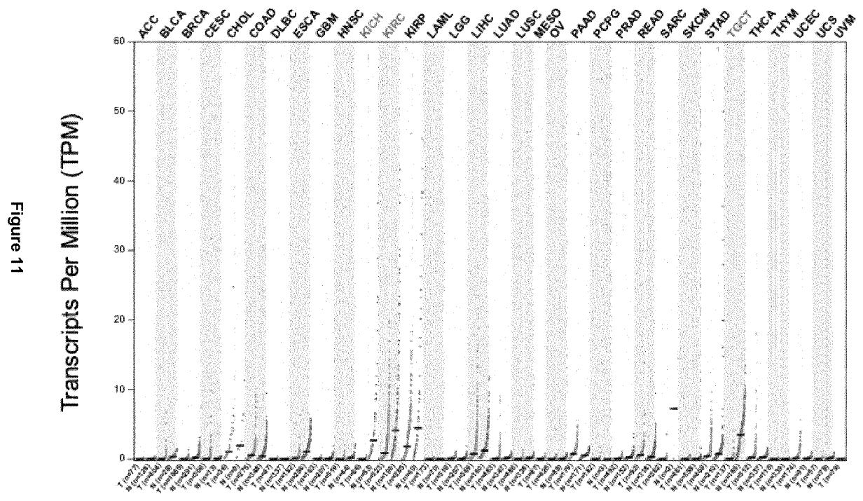

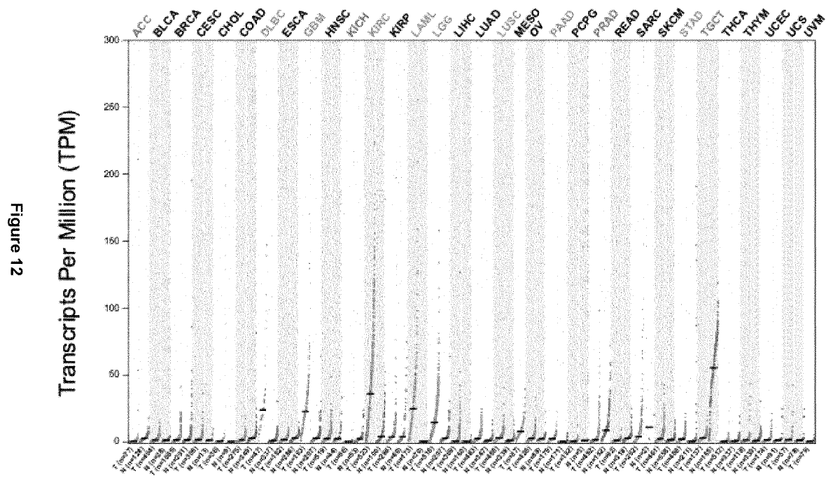

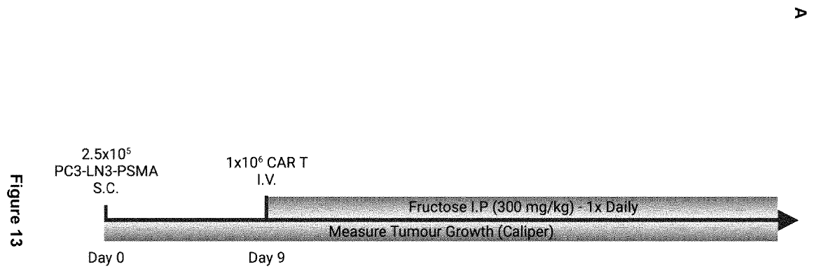

We engineered T-cells to express ADA2TM with a CAR (FIGS. 8-10). ADA2TM_4P28z was encoded by the nucleotide sequence SEQ ID NO: 17, with the resulting amino acid sequence being SEQ ID NO: 18. The conversion of adenosine to inosine will supply an ACS to glucose in order to fuel glycolysis and CAR T cell effector functions.

Additionally, adenosine is a potent immunosuppressive molecule in the TME. By facilitating conversion of TME adenosine to inosine this ACS may manipulate the TME to favour immune surveillance and cytotoxicity.

### Example 4: CAR T-Cells Engineered to Utilise an Alternative Carbon Source have Improved Anti-Tumour Functionality In Vivo

The in vivo anti-tumour functionality of T-cells genetically engineered to co-express a CAR and the transporter GLUT5 was considered. As explained above, expression of GLUT5 enables transport of fructose across the cell membrane, which can then be utilised as an ACS once within the cell.

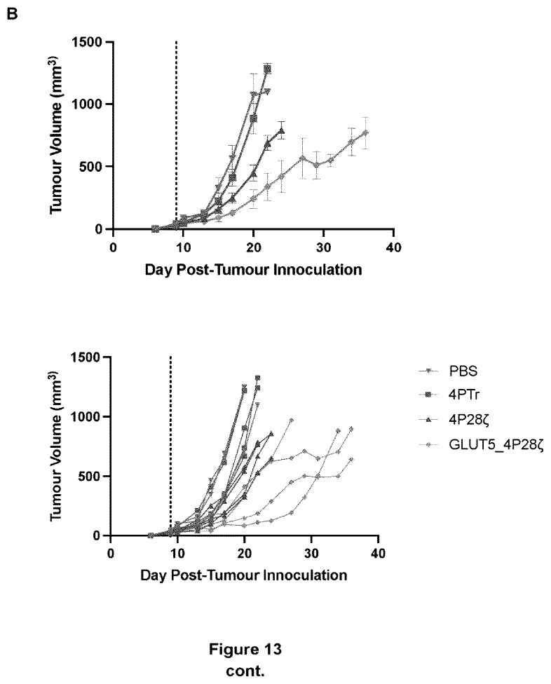

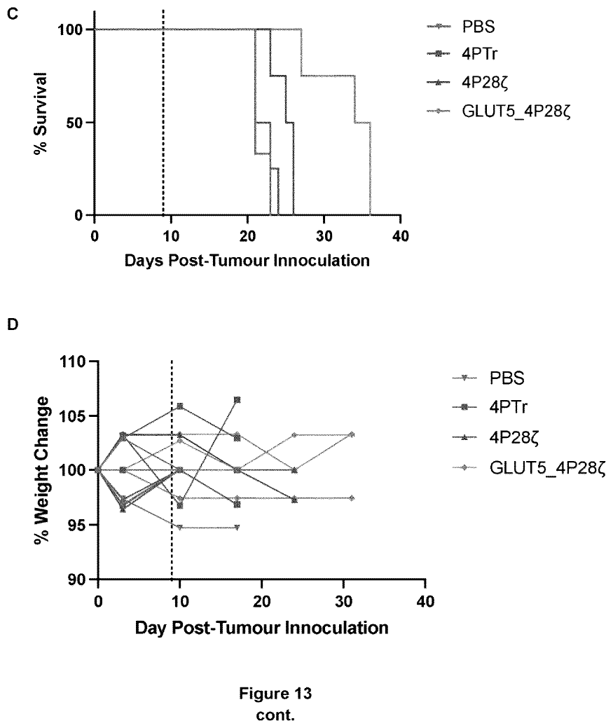

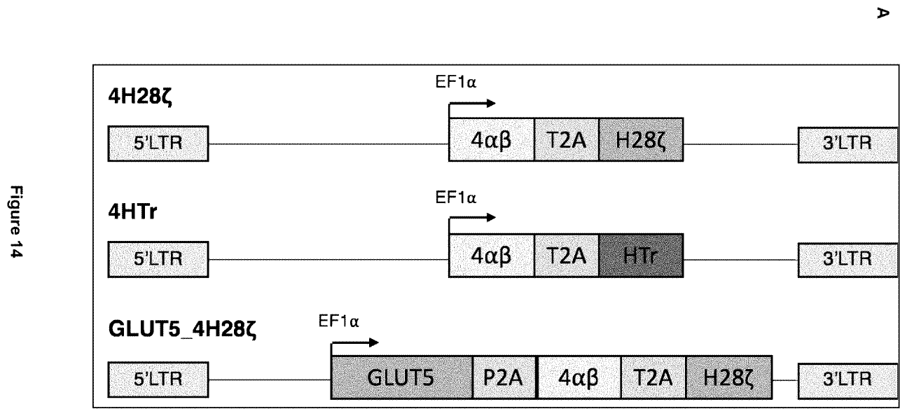

As FIG. 13A shows, NSG mice were subcutaneously inoculated with 2.5×10⁶ PC3-LN3-PSMA cells. Nine days after tumour engraftment, mice were intravenously injected with 1×10⁶ CAR⁺ T cells. After CAR T cell adoptive transfer, mice received daily intraperitoneal injections of fructose.

T-cells co-expressing GLUT5 and a CAR (GLUT5_4P28z) had improved anti-tumour functionality in vivo compared to T-cells expressing the CAR (4P28z) alone, a control signal CAR (4PTr) or control PBS. The smallest change in tumour volume over time was in mice which had received the GLUT5_4P28z T-cells (FIG. 13B), and mice which had received the GLUT5_4P28z T-cells had the longest survival time (FIG. 13C).

### Example 5: Co-Expression of Exogenous GLUT5 with Further Exogenous CARs or TCRs Enhances In Vitro Efficacy

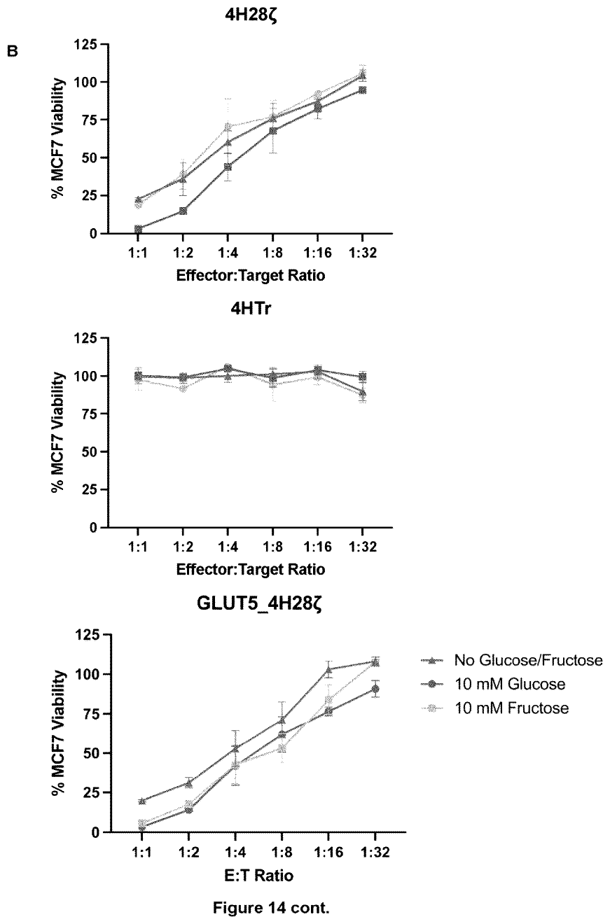

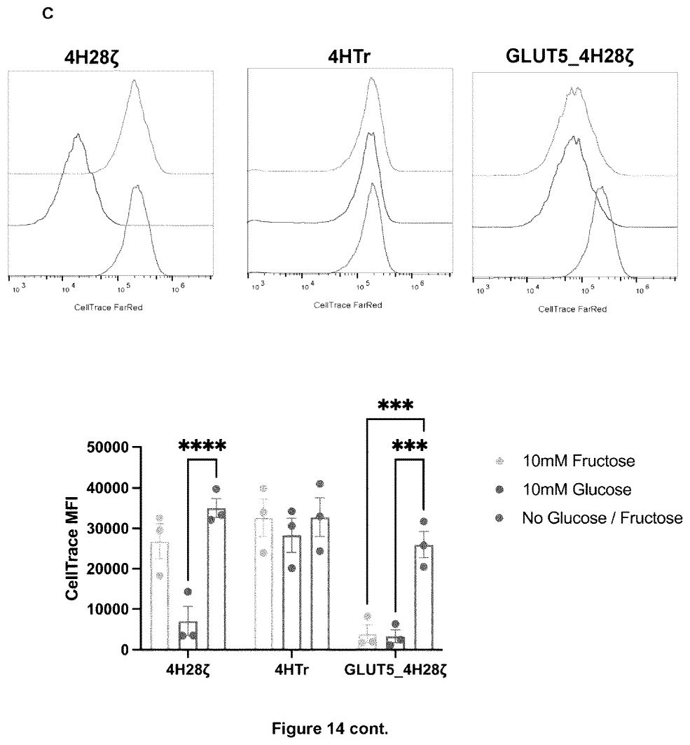

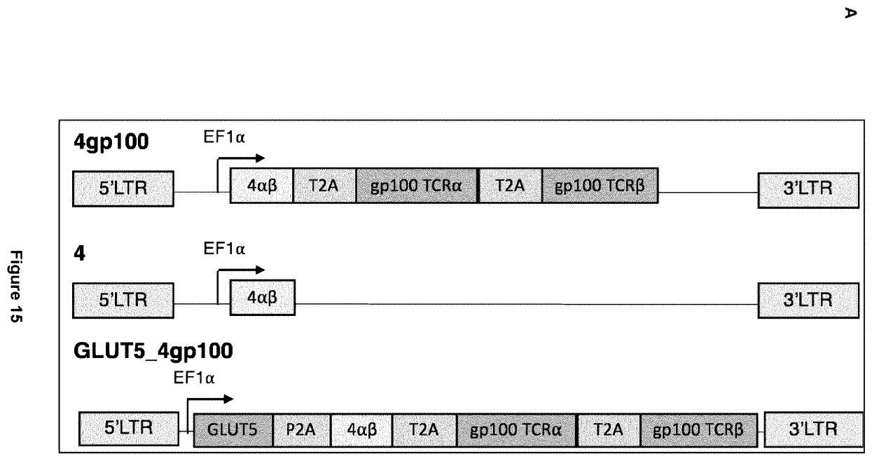

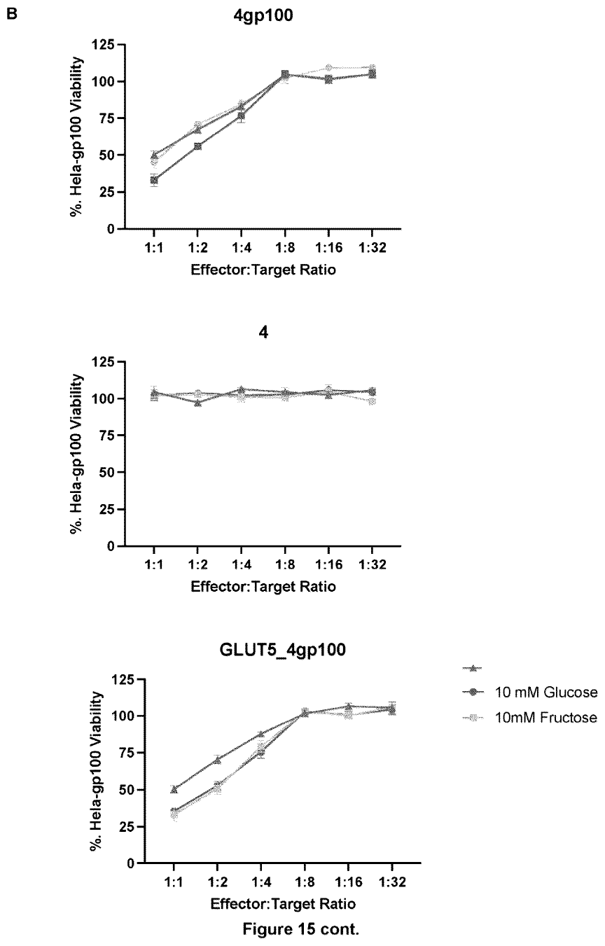

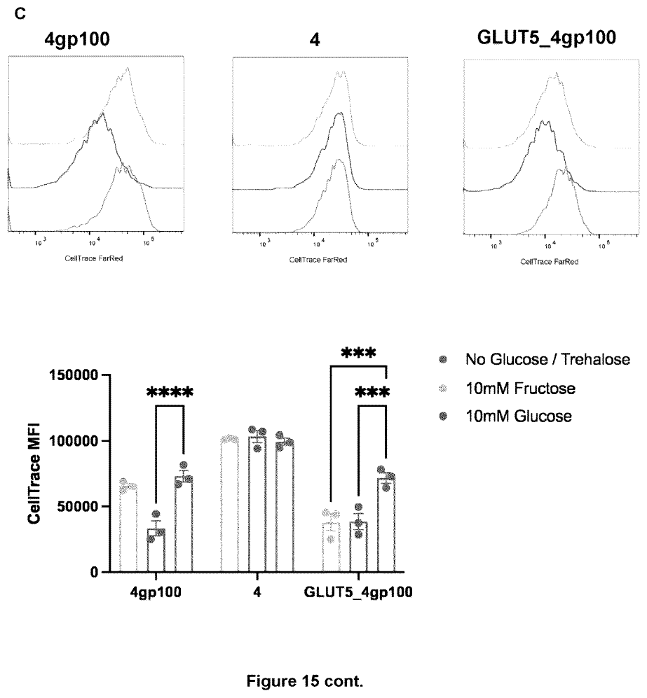

We engineered T-cells to express GLUT5 with a further CAR specific for HER-2 (4H28z) or a GP100-specific TCR (4gp100) (FIGS. 14 and 15). GLUT5_4H28z was encoded by the nucleotide sequence SEQ ID NO: 23, while GLUT5_4GP100 was encoded by the nucleotide sequence SEQ ID NO: 24.

The effect of expression of exogenous GLUT5 on the cytotoxicity of the HER-2 specific CAR or gp100-specific TCR T-cells is shown in FIGS. 14B and 15B, respectively. Exogenous expression of GLUT5 in anti-HER-2 CAR T-cells (GLUT5_4H28z) rescued cytotoxicity towards the HER-2-expressing MCF-7 breast cancer cells under hypoglycemic (no glucose) conditions in the presence of fructose (bottom graph of FIG. 14B compared to top graph of FIG. 14B). Likewise, exogenous expression of GLUT5 in anti-gp100 TCR T cells (GLUT5_4gp100) rescued cytotoxicity towards the HELA-gp-100 expressing cervical cancer cells under hypoglycemic (no glucose) conditions in the presence of fructose (bottom graph of FIG. 15B compared to top graph of FIG. 15B).

The co-expression of GLUT5 with the exogenous CAR or TCR also enabled the cells to proliferate in fructose-containing glucose-free media (FIGS. 14C and 15C). This level of proliferation was comparable to proliferation in media containing glucose. However, CAR or TCR T-cells which did not express GLUT5 were only able to proliferate in glucose containing media; for such cells, proliferation did not occur in fructose-containing glucose-free media.

LIST OF SEQUENCES
P28ζ Forward Primer
SEQ ID NO: 1
Ggctgcaggtccgatccaccggtcgccaccatggctctcccagtgactg
P28ζ Reverse Primer
SEQ ID NO: 2
Tcgagtcgacgactccggaacgaattctgattagcgagggggcagggc
PTr Forward Primer
SEQ ID NO: 3
ggctgcaggtccgatccaccggtcgccaccatggctctcccagtgactg
PTr Reverse Primer
SEQ ID NO: 4
Tcgagtcgacgactccggaacgaattctgattacttgctccgcacccagaagatg
T2A Forward Primer
SEQ ID NO: 5
aagcgcagcggctccggcgagggccggggcagcctgctg
T2A Reverse Primer
SEQ ID NO: 6
Gcagtcactgggagagccatgggtccggggttctcttccacgtcg
P2A Forward Primer
SEQ ID NO: 7
ggatctggagcaacaaacttctcact
P2A Reverse Primer
SEQ ID NO: 8
Aagcagtagggcagtcactgggagagccataggcccgggattctcctc
TREH Forward Primer
SEQ ID NO: 9
cgtgaaaactacccctaaaagctagcgccaccatgccagggaggacctg
TREH Reverse Primer
SEQ ID NO: 10
Aagtttgttgctccagatccccatggcaggaggctgagcaggagg
GLUT5 Forward Primer
SEQ ID NO: 11
ccctaaaagctagcgccaccatggagcaacaggatcagagcatgaagg
GLUT Reverse Primer
SEQ ID NO: 12
agtgagaagtttgttgctccagatccctgttccgaagtgacaggtggaagc
TREH 4P28z nucleotide sequence
SEQ ID NO: 13
ATGCCAGGGAGGACCTGGGAGCTGTGCCTGCTACTGCTGCTGGGGCTGGGACTGGGGTCCCAGGAGGCCC
TACCCCCACCCTGTGAGAGTGAGATTTACTGCCACGGGGAGCTCCTAAACCAAGTTCAAATGGCCAAGCT
CTACCAGGATGACAAGCAGTTTGTGGACATGCCACTGTCTATAGCTCCAGAACAAGTCCTGCAGACCTTC
ACTGAGCTGTCCAGGGACCACAATCACAGCATCCCCAGGGAGCAGCTGCAGGCGTTTGTCCACGAACACT
TCCAGGCCAAGGGGCAGGAGCTGCAGCCCTGGACCCCTGCAGACTGGAAAGACAGCCCCCAGTTCCTGCA
GAAGATTTCAGATGCCAAACTGCGTGCCTGGGCAGGGCAGCTGCATCAGCTCTGGAAGAAGCTGGGGAAG
AAGATGAAGCCAGAGGTTCTCAGCCACCCTGAGCGGTTCTCTCTCATCTACTCAGAACATCCCTTCATTG
TGCCTGGCGGTCGCTTTGTTGAGTTCTACTACTGGGACTCCTACTGGGTCATGGAGGGTCTGCTCCTCTC
AGAGATGGCTGAGACGGTGAAGGGCATGCTGCAGAACTTCTTGGACCTGGTGAAAACCTATGGGCATGTC
CCCAATGGTGGGCGCGTGTACTACCTGCAGCGGAGCCAGCCCCCACTCTTGACCCTCATGATGGATTGCT
ACTTGACTCACACCAATGACACCGCCTTTCTACAGGAAAACATTGAAACACTAGCCTTGGAATTGGACTT
TTGGACCAAGAACAGGACTGTCTCTGTGAGCTTGGAGGGAAAGAACTACCTCCTGAATCGCTATTATGTC
CCTTATGGGGGACCCAGGCCTGAGTCCTACAGCAAAGATGTGGAGTTGGCTGACACCTTGCCAGAAGGAG
ACCGGGAGGCTCTGTGGGCTGAGCTCAAGGCTGGGGCTGAGTCTGGCTGGGACTTCTCTTCACGCTGGCT
CATTGGAGGCCCAAACCCCAACTCGCTTAGCGGCATCCGAACAAGCAAACTGGTGCCTGTTGACCTGAAT
GCCTTCCTATGCCAAGCAGAGGAGCTGATGAGCAACTTCTATTCCAGGCTGGGGAACGACTCCCAGGCCA
CGAAGTACAGAATCCTGCGGTCGCAGCGCTTGGCCGCCCTGAACACAGTCCTGTGGGATGAGCAGACCGG
AGCCTGGTTCGATTACGACCTTGAGAAGAAGAAGAAAAACCGGGAGTTTTACCCATCCAACCTCACTCCA
CTCTGGGCCGGGTGTTTCTCTGACCCTGGCGTGGCGGACAAGGCTCTGAAATACCTGGAGGACAACCGGA
TCCTGACTTACCAGTATGGGATCCCGACCTCTCTCCAGAAGACAGGCCAGCAGTGGGATTTCCCCAATGC
CTGGGCCCCCCTGCAGGACCTGGTCATCAGAGGCCTGGCCAAGGCACCTTTACGTCGGGCCCAGGAAGTG
GCTTTCCAGCTGGCTCAGAATTGGATCCGAACCAATTTTGATGTCTACTCGCAGAAGTCAGCCATGTATG
AGAAGTATGACGTCAGCAACGGTGGACAGCCCGGTGGGGGAGGAGAATATGAAGTTCAGGAGGGATTTGG
CTGGACGAATGGCGTGGTCCTGATGCTGCTGGACCGCTATGGTGACCGGCTGACCTCAGGGGCCAAGCTG
GCTTTCCTGGAGCCCCACTGCCTGGCGGCCACCCTTCTGCCCAGCCTCCTGCTCAGCCTCCTGCCATGGG
GATCTGGAGCAACAAACTTCTCACTACTCAAACAAGCAGGTGACGTGGAGGAGAATCCCGGGCCTATGGG
CTGGCTGTGTTCCGGCCTGCTGTTTCCTGTGTCCTGTCTGGTGCTGCTGCAGGTGGCCAGCTCCGGGAAC
ATGAAAGTGCTGCAGGAGCCCACATGTGTGTCCGACTACATGTCCATCTCTACATGTGAGTGGAAGATGA
ACGGCCCCACAAACTGCTCTACCGAGCTGCGGCTGCTGTACCAGCTGGTGTTTCTGCTGAGCGAGGCCCA
CACCTGTATCCCAGAAAATAATGGCGGGGCCGGGTGTGTGTGCCACCTGCTGATGGATGACGTGGTGTCT
GCCGACAATTACACCCTGGACCTGTGGGCCGGACAGCAGCTGCTGTGGAAGGGGTCCTTCAAACCCTCTG
AGCACGTGAAGCCAAGGGCCCCCGGCAACCTGACAGTGCACACCAACGTGTCTGATACACTGCTGCTGAC
ATGGAGCAATCCATACCCTCCTGACAACTACCTGTACAACCACCTGACCTACGCCGTGAATATCTGGAGC
GAAAATGATCCTGCCGACTTTCGGATTTACAATGTGACCTATCTGGAGCCCTCCCTGAGAATTGCCGCCT
CTACCCTGAAATCTGGAATCTCCTACCGCGCCAGGGTGCGGGCCTGGGCCCAGTGTTACAACACCACCTG
GTCTGAGTGGAGCCCAAGCACCAAGTGGCACAATTCTTATCGGGAGCCTTTTGAGCAGCACCTGATCCCC
TGGCTGGGACACCTGCTGGTGGGGCTGTCTGGCGCCTTTGGCTTCATCATTCTGGTGTACCTGCTGATCA
ACTGTAGGAATACAGGCCCTTGGCTGAAGAAGGTGCTGAAGTGTAACACCCCCGACCCCTCTAAGTTCTT
CAGCCAGCTGTCCTCTGAACACGGGGGAGATGTGCAGAAGTGGCTGTCCAGCCCTTTCCCATCCAGCTCC
TTTAGCCCCGGGGGCCTGGCCCCTGAGATCTCTCCACTGGAAGTGCTGGAGCGGGACAAGGTGACCCAGC
TGCTGCTGCAGCAGGACAAGGTGCCAGAACCCGCCTCCCTGAGCTCCAACCACAGCCTGACATCTTGCTT
TACAAATCAGGGATACTTCTTCTTCCACCTGCCCGATGCCCTGGAGATCGAGGCCTGCCAGGTGTACTTC
ACCTACGATCCCTACTCTGAGGAAGACCCAGATGAGGGCGTGGCCGGGGCCCCAACCGGGTCCAGCCCAC
AGCCACTGCAGCCACTGTCCGGCGAAGATGACGCCTACTGCACATTCCCTTCCAGGGATGACCTGCTGCT
GTTCAGCCCATCTCTGCTGGGCGGACCCTCTCCTCCAAGCACAGCCCCAGGGGGATCCGGCGCCGGGGAA
GAGAGGATGCCCCCTAGCCTGCAGGAGCGCGTGCCCAGAGACTGGGACCCCCAGCCCCTGGGCCCTCCAA
CCCCTGGGGTGCCCGACCTGGTGGACTTCCAGCCTCCACCCGAGCTGGTGCTGAGGGAGGCCGGCGAAGA
GGTGCCCGACGCCGGCCCCCGGGAGGGCGTGTCCTTCCCTTGGTCCAGACCTCCAGGACAGGGCGAGTTC
CGCGCCCTGAACGCCAGGCTGCCTCTGAACACCGATGCCTACCTGTCTCTGCAGGAACTGCAGGGCCAGG
ACCCAACCCACCTGGTGCGGAGAAAGCGCAGCGGCTCCGGCGAGGGCCGGGGCAGCCTGCTGACCTGCGG
CGACGTGGAAGAGAACCCCGGACCCATGGCTCTCCCAGTGACTGCCCTACTGCTTCCCCTAGCGCTTCTC
CTGCATGCAGAGGTGCAGCTGCAGCAGTCAGGACCTGAACTGGTGAAGCCTGGGACTTCAGTGAGGATAT
CCTGCAAGACTTCTGGATACACATTCACTGAATATACCATACACTGGGTGAAGCAGAGCCATGGAAAGAG
CCTTGAGTGGATTGGAAACATCAATCCTAACAATGGTGGTACCACCTACAATCAGAAGTTCGAGGACAAG
GCCACATTGACTGTAGACAAGTCCTCCAGTACAGCCTACATGGAGCTCCGCAGCCTAACATCTGAGGATT
CTGCAGTCTATTATTGTGCAGCTGGTTGGAACTTTGACTACTGGGGCCAAGGGACCACGGTCACCGTCTC
CTCAGGTGGAGGTGGATCAGGTGGAGGTGGATCTGGTGGAGGTGGATCTGACATTGTGATGACCCAGTCT
CACAAATTCATGTCCACATCAGTAGGAGACAGGGTCAGCATCATCTGTAAGGCCAGTCAAGATGTGGGTA
CTGCTGTAGACTGGTATCAACAGAAACCAGGACAATCTCCTAAACTACTGATTTATTGGGCATCCACTCG
GCACACTGGAGTCCCTGATCGCTTCACAGGCAGTGGATCTGGGACAGACTTCACTCTCACCATTACTAAT
GTTCAGTCTGAAGACTTGGCAGATTATTTCTGTCAGCAATATAACAGCTATCCCCTCACGTTCGGTGCTG
GGACCATGCTGGACCTGAAACGGGCGGCCGCCATCGAGGTGGAGCAGAAGCTGATCAGCGAGGAGGACCT
GCTGGACAACGAGAAGAGCAACGGCACCATCATCCACGTGAAGGGCAAGCACCTGTGCCCCAGCCCCCTG
TTCCCCGGCCCCAGCAAGCCCTTCTGGGTGCTGGTGGTGGTGGGCGGCGTGCTGGCCTGCTACAGCCTGC
TGGTGACCGTGGCCTTCATCATCTTCTGGGTGCGGAGCAAGCGGAGCCGGCTGCTGCACAGCGACTACAT
GAACATGACCCCCCGGCGGCCTGGGCCCACCCGCAAGCATTACCAGCCCTATGCCCCACCACGCGACTTC
GCAGCCTATCGCTCCAGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAGAGACGTGGCCGGGA
CCCTGAGATGGGGGGAAAGCCGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAGGGGCAAGGGGCACGATGGCC
TTTACCAGGGTCTCAGTACAGCCACCAAGGACACCTACGACGCCCTTCACATGCAGGCCCTGCCCCCTCG
C
TREH 4P28z amino acid sequence
SEQ ID NO: 14
MPGRTWELCLLLLLGLGLGSQEALPPPCESEIYCHGELLNQVQMAKLYQDDKQFVDMPLSIAPEQVLQTF
TELSRDHNHSIPREQLQAFVHEHFQAKGQELQPWTPADWKDSPQFLQKISDAKLRAWAGQLHQLWKKLGK
KMKPEVLSHPERFSLIYSEHPFIVPGGRFVEFYYWDSYWVMEGLLLSEMAETVKGMLQNFLDLVKTYGHV
PNGGRVYYLQRSQPPLLTLMMDCYLTHTNDTAFLQENIETLALELDFWTKNRTVSVSLEGKNYLLNRYYV
PYGGPRPESYSKDVELADTLPEGDREALWAELKAGAESGWDFSSRWLIGGPNPNSLSGIRTSKLVPVDLN
AFLCQAEELMSNFYSRLGNDSQATKYRILRSQRLAALNTVLWDEQTGAWFDYDLEKKKKNREFYPSNLTP
LWAGCFSDPGVADKALKYLEDNRILTYQYGIPTSLQKTGQQWDFPNAWAPLQDLVIRGLAKAPLRRAQEV
AFQLAQNWIRTNFDVYSQKSAMYEKYDVSNGGQPGGGGEYEVQEGFGWTNGVVLMLLDRYGDRLTSGAKL
AFLEPHCLAATLLPSLLLSLLPWGSGATNFSLLKQAGDVEENPGPMGWLCSGLLFPVSCLVLLQVASSGN
MKVLQEPTCVSDYMSISTCEWKMNGPTNCSTELRLLYQLVFLLSEAHTCIPENNGGAGCVCHLLMDDVVS
ADNYTLDLWAGQQLLWKGSFKPSEHVKPRAPGNLTVHTNVSDTLLLTWSNPYPPDNYLYNHLTYAVNIWS
ENDPADFRIYNVTYLEPSLRIAASTLKSGISYRARVRAWAQCYNTTWSEWSPSTKWHNSYREPFEQHLIP
WLGHLLVGLSGAFGFIILVYLLINCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCFTNQGYFFFHLPDALEIEACQVYF
TYDPYSEEDPDEGVAGAPTGSSPQPLQPLSGEDDAYCTFPSRDDLLLFSPSLLGGPSPPSTAPGGSGAGE
ERMPPSLQERVPRDWDPQPLGPPTPGVPDLVDFQPPPELVLREAGEEVPDAGPREGVSFPWSRPPGQGEF
RALNARLPLNTDAYLSLQELQGQDPTHLVRRKRSGSGEGRGSLLTCGDVEENPGPMALPVTALLLPLALL
LHAEVQLQQSGPELVKPGTSVRISCKTSGYTFTEYTIHWVKQSHGKSLEWIGNINPNNGGTTYNQKFEDK
ATLTVDKSSSTAYMELRSLTSEDSAVYYCAAGWNFDYWGQGTTVTVSSGGGGSGGGGSGGGGSDIVMTQS
HKFMSTSVGDRVSIICKASQDVGTAVDWYQQKPGQSPKLLIYWASTRHTGVPDRFTGSGSGTDFTLTITN
VQSEDLADYFCQQYNSYPLTFGAGTMLDLKRAAAIEVEQKLISEEDLLDNEKSNGTIIHVKGKHLCPSPL
FPGPSKPFWVLVVVGGVLACYSLLVTVAFIIFWVRSKRSRLLHSDYMNMTPRRPGPTRKHYQPYAPPRDF
AAYRSRVKFSRSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
GLUT5 4P28z nucleotide sequence
SEQ ID NO: 15
ttctttcctgcgttatcccctgattctgtggataaccgtattaccgcctttgagtgagctgataccgctc
gccgcagccgaacgaccgagcgcagcgagtcagtgagcgaggaagcggaagagcgcccaatacgcaaacc
gcctctccccgcgcgttggccgattcattaatgcagctggcacgacaggtttcccgactggaaagcgggc
agtgagcgcaacgcaattaatgtgagttagctcactcattaggcaccccaggctttacactttatgcttc
cggctcgtatgttgtgtggaattgtgagcggataacaatttcacacaggaaacagctatgaccatgatta
cgccaagcgcgcaattaaccctcactaaagggaacaaaagctggagctgcaagcttaatgtagtcttatg
caatactcttgtagtcttgcaacatggtaacgatgagttagcaacatgccttacaaggagagaaaaagca
ccgtgcatgccgattggtggaagtaaggtggtacgatcgtgccttattaggaaggcaacagacgggtctg
acatggattggacgaaccactgaattgccgcattgcagagatattgtatttaagtgcctagctcgataca
taaacgggtctctctggttagaccagatctgagcctgggagctctctggctaactagggaacccactgct
taagcctcaataaagcttgccttgagtgcttcaagtagtgtgtgcccgtctgttgtgtgactctggtaac
tagagatccctcagacccttttagtcagtgtggaaaatctctagcagtggcgcccgaacagggacttgaa
agcgaaagggaaaccagaggagctctctcgacgcaggactcggcttgctgaagcgcgcacggcaagaggc
gaggggcggcgactggtgagtacgccaaaaattttgactagcggaggctagaaggagagagatgggtgcg
agagcgtcagtattaagcgggggagaattagatcgcgatgggaaaaaattcggttaaggccagggggaaa
gaaaaaatataaattaaaacatatagtatgggcaagcagggagctagaacgattcgcagttaatcctggc
ctgttagaaacatcagaaggctgtagacaaatactgggacagctacaaccatcccttcagacaggatcag
aagaacttagatcattatataatacagtagcaaccctctattgtgtgcatcaaaggatagagataaaaga
caccaaggaagctttagacaagatagaggaagagcaaaacaaaagtaagaccaccgcacagcaagcggcc
gctgatcttcagacctggaggaggagatatgagggacaattggagaagtgaattatataaatataaagta
gtaaaaattgaaccattaggagtagcacccaccaaggcaaagagaagagtggtgcagagagaaaaaagag
cagtgggaataggagctttgttccttgggttcttgggagcagcaggaagcactatgggcgcagcgtcaat
gacgctgacggtacaggccagacaattattgtctggtatagtgcagcagcagaacaatttgctgagggct
attgaggcgcaacagcatctgttgcaactcacagtctggggcatcaagcagctccaggcaagaatcctgg
ctgtggaaagatacctaaaggatcaacagctcctggggatttggggttgctctggaaaactcatttgcac
cactgctgtgccttggaatgctagttggagtaataaatctctggaacagatttggaatcacacgacctgg
atggagtgggacagagaaattaacaattacacaagcttaatacactccttaattgaagaatcgcaaaacc
agcaagaaaagaatgaacaagaattattggaattagataaatgggcaagtttgtggaattggtttaacat
aacaaattggctgtggtatataaaattattcataatgatagtaggaggcttggtaggtttaagaatagtt
tttgctgtactttctatagtgaatagagttaggcagggatattcaccattatcgtttcagacccacctcc
caaccccgaggggacccgacaggcccgaaggaatagaagaagaaggtggagagagagacagagacagatc
cattcgattagtgaacggatcggcactgcgtgcgccaattctgcagacaaatggcagtattcatccacaa
ttttaaaagaaaaggggggattggggggtacagtgcaggggaaagaatagtagacataatagcaacagac
atacaaactaaagaattacaaaaacaaattacaaaaattcaaaattttcgggtttattacagggacagca
gagatccagtttggttaattaacccgtgtcggctccagatctgctccggtgcccgtcagtgggcagagcg
cacatcgcccacagtccccgagaagttggggggaggggtcggcaattgaaccggtgcctagagaaggtgg
cgcggggtaaactgggaaagtgatgtcgtgtactggctccgcctttttcccgagggtgggggagaaccgt
atataagtgcagtagtcgccgtgaacgttctttttcgcaacgggtttgccgccagaacacaggtaagtgc
cgtgtgtggttcccgcgggcctggcctctttacgggttatggcccttgcgtgccttgaattacttccacg
cccctggctgcagtacgtgattcttgatcccgagcttcgggttggaagtgggtgggagagttcgaggcct
tgcgcttaaggagccccttcgcctcgtgcttgagttgaggcctggcttgggcgctggggccgccgcgtgc
gaatctggtggcaccttcgcgcctgtctcgctgctttcgataagtctctagccatttaaaatttttgatg
acctgctgcgacgctttttttctggcaagatagtcttgtaaatgcgggccaagatctgcacactggtatt
tcggtttttggggccgcgggcggcgacggggcccgtgcgtcccagcgcacatgttcggcgaggcggggcc
tgcgagcgcggccaccgagaatcggacgggggtagtctcaagctggccggcctgctctggtgcctggcct
cgcgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggggg
tgagtcacccacacaaaggaaaagggcctttccgtcctcagccgtcgcttcatgtgactccacggagtac
cgggcgccgtccaggcacctcgattagttctcgagcttttggagtacgtcgtctttaggttggggggagg
ggttttatgcgatggagtttccccacactgagtgggtggagactgaagttaggccagcttggcacttgat
gtaattctccttggaatttgccctttttgagtttggatcttggttcattctcaagcctcagacagtggtt
caaagtttttttcttccatttcaggtgtcgtgaaaactacccctaaaaGCTAGCgccaccATGGAGCAAC
AGGATCAGAGCATGAAGGAAGGGAGGCTGACGCTTGTGCTTGCCCTGGCAACCCTGATAGCTGCCTTTGG
GTCATCCTTCCAGTATGGGTACAACGTGGCTGCTGTCAACTCCCCAGCACTGCTCATGCAACAATTTTAC
AATGAGACTTACTATGGTAGGACCGGTGAATTCATGGAAGACTTCCCCTTGACGTTGCTGTGGTCTGTAA
CCGTGTCCATGTTTCCATTTGGAGGGTTTATCGGATCCCTCCTGGTCGGCCCCTTGGTGAATAAATTTGG
CAGAAAAGGGGCCTTGCTGTTCAACAACATATTTTCTATCGTGCCTGCGATCTTAATGGGATGCAGCAGA
GTCGCCACATCATTTGAGCTTATCATTATTTCCAGACTTTTGGTTGGAATATGTGCAGGTGTATCTTCCA
ACGTGGTCCCCATGTACTTAGGGGAGCTGGCCCCTAAAAACCTGCGGGGGGCTCTCGGGGTGGTGCCCCA
GCTCTTCATCACTGTTGGCATCCTTGTGGCCCAGATCTTTGGTCTTCGGAATCTCCTTGCAAACGTAGAT
GGCTGGCCGATCCTGCTGGGGCTGACCGGGGTCCCCGCGGCGCTGCAGCTCCTTCTGCTGCCCTTCTTCC
CCGAGAGCCCCAGGTACCTGCTGATTCAGAAGAAAGACGAAGCGGCCGCCAAGAAAGCCCTACAGACGCT
GCGCGGCTGGGACTCTGTGGACAGGGAGGTGGCCGAGATCCGGCAGGAGGATGAGGCAGAGAAGGCCGCG
GGCTTCATCTCCGTGCTGAAGCTGTTCCGGATGCGCTCGCTGCGCTGGCAGCTGCTGTCCATCATCGTCC
TCATGGGCGGCCAGCAGCTGTCGGGCGTCAACGCTATCTACTACTACGCGGACCAGATCTACCTGAGCGC
CGGCGTGCCGGAGGAGCACGTGCAGTACGTGACGGCCGGCACCGGGGCCGTGAACGTGGTCATGACCTTC
TGCGCCGTGTTCGTGGTGGAGCTCCTGGGTCGGAGGCTGCTGCTGCTGCTGGGCTTCTCCATCTGCCTCA
TAGCCTGCTGCGTGCTCACTGCAGCTCTGGCACTGCAGGACACAGTGTCCTGGATGCCATACATCAGCAT
CGTCTGTGTCATCTCCTACGTCATAGGACATGCCCTCGGGCCCAGTCCCATACCCGCGCTGCTCATCACT
GAGATCTTCCTGCAGTCCTCTCGGCCATCTGCCTTCATGGTGGGGGGCAGTGTGCACTGGCTCTCCAACT
TCACCGTGGGCTTGATCTTCCCGTTCATCCAGGAGGGCCTCGGCCCGTACAGCTTCATTGTCTTCGCCGT
GATCTGCCTCCTCACCACCATCTACATCTTCTTGATTGTCCCGGAGACCAAGGCCAAGACGTTCATAGAG
ATCAACCAGATTTTCACCAAGATGAATAAGGTGTCTGAAGTGTACCCGGAAAAGGAGGAACTGAAAGAGC
TTCCACCTGTCACTTCGGAACAGGGATCTGGAGCAACAAACTTCTCACTACTCAAACAAGCAGGTGACGT
GGAGGAGAATCCCGGGCCTATGGGCTGGCTGTGTTCCGGCCTGCTGTTTCCTGTGTCCTGTCTGGTGCTG
CTGCAGGTGGCCAGCTCCGGGAACATGAAAGTGCTGCAGGAGCCCACATGTGTGTCCGACTACATGTCCA
TCTCTACATGTGAGTGGAAGATGAACGGCCCCACAAACTGCTCTACCGAGCTGCGGCTGCTGTACCAGCT
GGTGTTTCTGCTGAGCGAGGCCCACACCTGTATCCCAGAAAATAATGGCGGGGCCGGGTGTGTGTGCCAC
CTGCTGATGGATGACGTGGTGTCTGCCGACAATTACACCCTGGACCTGTGGGCCGGACAGCAGCTGCTGT
GGAAGGGGTCCTTCAAACCCTCTGAGCACGTGAAGCCAAGGGCCCCCGGCAACCTGACAGTGCACACCAA
CGTGTCTGATACACTGCTGCTGACATGGAGCAATCCATACCCTCCTGACAACTACCTGTACAACCACCTG
ACCTACGCCGTGAATATCTGGAGCGAAAATGATCCTGCCGACTTTCGGATTTACAATGTGACCTATCTGG
AGCCCTCCCTGAGAATTGCCGCCTCTACCCTGAAATCTGGAATCTCCTACCGCGCCAGGGTGCGGGCCTG
GGCCCAGTGTTACAACACCACCTGGTCTGAGTGGAGCCCAAGCACCAAGTGGCACAATTCTTATCGGGAG
CCTTTTGAGCAGCACCTGATCCCCTGGCTGGGACACCTGCTGGTGGGGCTGTCTGGCGCCTTTGGCTTCA
TCATTCTGGTGTACCTGCTGATCAACTGTAGGAATACAGGCCCTTGGCTGAAGAAGGTGCTGAAGTGTAA
CACCCCCGACCCCTCTAAGTTCTTCAGCCAGCTGTCCTCTGAACACGGGGGAGATGTGCAGAAGTGGCTG
TCCAGCCCTTTCCCATCCAGCTCCTTTAGCCCCGGGGGCCTGGCCCCTGAGATCTCTCCACTGGAAGTGC
TGGAGCGGGACAAGGTGACCCAGCTGCTGCTGCAGCAGGACAAGGTGCCAGAACCCGCCTCCCTGAGCTC
CAACCACAGCCTGACATCTTGCTTTACAAATCAGGGATACTTCTTCTTCCACCTGCCCGATGCCCTGGAG
ATCGAGGCCTGCCAGGTGTACTTCACCTACGATCCCTACTCTGAGGAAGACCCAGATGAGGGCGTGGCCG
GGGCCCCAACCGGGTCCAGCCCACAGCCACTGCAGCCACTGTCCGGCGAAGATGACGCCTACTGCACATT
CCCTTCCAGGGATGACCTGCTGCTGTTCAGCCCATCTCTGCTGGGCGGACCCTCTCCTCCAAGCACAGCC
CCAGGGGGATCCGGCGCCGGGGAAGAGAGGATGCCCCCTAGCCTGCAGGAGCGCGTGCCCAGAGACTGGG
ACCCCCAGCCCCTGGGCCCTCCAACCCCTGGGGTGCCCGACCTGGTGGACTTCCAGCCTCCACCCGAGCT
GGTGCTGAGGGAGGCCGGCGAAGAGGTGCCCGACGCCGGCCCCCGGGAGGGCGTGTCCTTCCCTTGGTCC
AGACCTCCAGGACAGGGCGAGTTCCGCGCCCTGAACGCCAGGCTGCCTCTGAACACCGATGCCTACCTGT
CTCTGCAGGAACTGCAGGGCCAGGACCCAACCCACCTGGTGCGGAGAAAGCGCAGCGGCTCCGGCGAGGG
CCGGGGCAGCCTGCTGACCTGCGGCGACGTGGAAGAGAACCCCGGACCCATGGCTCTCCCAGTGACTGCC
CTACTGCTTCCCCTAGCGCTTCTCCTGCATGCAGAGGTGCAGCTGCAGCAGTCAGGACCTGAACTGGTGA
AGCCTGGGACTTCAGTGAGGATATCCTGCAAGACTTCTGGATACACATTCACTGAATATACCATACACTG
GGTGAAGCAGAGCCATGGAAAGAGCCTTGAGTGGATTGGAAACATCAATCCTAACAATGGTGGTACCACC
TACAATCAGAAGTTCGAGGACAAGGCCACATTGACTGTAGACAAGTCCTCCAGTACAGCCTACATGGAGC
TCCGCAGCCTAACATCTGAGGATTCTGCAGTCTATTATTGTGCAGCTGGTTGGAACTTTGACTACTGGGG
CCAAGGGACCACGGTCACCGTCTCCTCAGGTGGAGGTGGATCAGGTGGAGGTGGATCTGGTGGAGGTGGA
TCTGACATTGTGATGACCCAGTCTCACAAATTCATGTCCACATCAGTAGGAGACAGGGTCAGCATCATCT
GTAAGGCCAGTCAAGATGTGGGTACTGCTGTAGACTGGTATCAACAGAAACCAGGACAATCTCCTAAACT
ACTGATTTATTGGGCATCCACTCGGCACACTGGAGTCCCTGATCGCTTCACAGGCAGTGGATCTGGGACA
GACTTCACTCTCACCATTACTAATGTTCAGTCTGAAGACTTGGCAGATTATTTCTGTCAGCAATATAACA
GCTATCCCCTCACGTTCGGTGCTGGGACCATGCTGGACCTGAAACGGGCGGCCGCCATCGAGGTGGAGCA
GAAGCTGATCAGCGAGGAGGACCTGCTGGACAACGAGAAGAGCAACGGCACCATCATCCACGTGAAGGGC
AAGCACCTGTGCCCCAGCCCCCTGTTCCCCGGCCCCAGCAAGCCCTTCTGGGTGCTGGTGGTGGTGGGCG
GCGTGCTGGCCTGCTACAGCCTGCTGGTGACCGTGGCCTTCATCATCTTCTGGGTGCGGAGCAAGCGGAG
CCGGCTGCTGCACAGCGACTACATGAACATGACCCCCCGGCGGCCTGGGCCCACCCGCAAGCATTACCAG
CCCTATGCCCCACCACGCGACTTCGCAGCCTATCGCTCCAGAGTGAAGTTCAGCAGGAGCGCAGACGCCC
CCGCGTACCAGCAGGGCCAGAACCAGCTCTATAACGAGCTCAATCTAGGACGAAGAGAGGAGTACGATGT
TTTGGACAAGAGACGTGGCCGGGACCCTGAGATGGGGGGAAAGCCGAGAAGGAAGAACCCTCAGGAAGGC
CTGTACAATGAACTGCAGAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGGACACCTACGACGCCCT
TCACATGCAGGCCCTGCCCCCTCGCTAAtcagaattcgttccggagtcgtcgactcgacaatcaacctct
ggattacaaaatttgtgaaagattgactggtattcttaactaTGTTGCTCCTTTTACGCTATGtggatac
gctgctttaatgcctttgtatcatgctattgcttcccgtatggctttcattttctcctccttgtataaat
cctggttgctgtctctttatgaggagttgtggcccgttgtcaggcaacgtggcgtggtgtgcactgtgtt
tgctgacgcaacccccactggttggggcattgccaccacctgtcagctcctttccgggactttcgctttc
cccctccctattgccacggcggaactcatcgccgcctgccttgcccgctgctggacaggggctcggctgt
tgggcactgacaattccgtggtgttgtcggggaagctgacgtcctttccatggctgctcgcctgtgttgc
cacctggattctgcgcgggacgtccttctgctacgtcccttcggccctcaatccagcggaccttccttcc
cgcggcctgctgccggctctgcggcctcttccgcatcttcgccttcgccctcagacgagtcggatctccc
tttgggccgcctccccgcctggaattaattcgagctcggtacctttaagaccaatgacttacaaggcagc
tgtagatcttagccactttttaaaagaaaaggggggactggaagggctacgtaactcccaacgaagacaa
gatctgctttttgcttgtactgggtctctctggttagaccagatctgagcctgggagctctctggctaac
tagggaacccactgcttaagcctcaataaagcttgccttgagtgcttcaagtagtgtgtgcccgtctgtt
gtgtgactctggtaactagagatccctcagacccttttagtcagtgtggaaaatctctagcagtagtagt
tcatgtcatcttattattcagtatttataacttgcaaagaaatgaatatcagagagtgagaggaacttgt
ttattgcagcttataatggttacaaataaagcaatagcatcacaaatttcacaaataaagcatttttttc
actgcattctagttgtggtttgtccaaactcatcaatgtatcttatcatgtctggctctagctatcccgc
ccctaactccgcccatcccgcccctaactccgcccagttccgcccattctccgccccatggctgactaat
tttttttatttatgcagaggccgaggccgcctcggcctctgagctattccagaagtagtgaggaggcttt
tttggaggcctagggacgtacccaattcgccctatagtgagtcgtattacgcgcgctcactggccgtcgt
tttacaacgtcgtgactgggaaaaccctggcgttacccaacttaatcgccttgcagcacatccccctttc
gccagctggcgtaatagcgaagaggcccgcaccgatcgcccttcccaacagttgcgcagcctgaatggcg
aatgggacgcgccctgtagcggcgcattaagcgcggcgggtgtggtggttacgcgcagcgtgaccgctac
acttgccagcgccctagcgcccgctcctttcgctttcttcccttcctttctcgccacgttcgccggcttt
ccccgtcaagctctaaatcgggggctccctttagggttccgatttagtgctttacggcacctcgacccca
aaaaacttgattagggtgatggttcacgtagtgggccatcgccctgatagacggtttttcgccctttgac
gttggagtccacgttctttaatagtggactcttgttccaaactggaacaacactcaaccctatctcggtc
tattcttttgatttataagggattttgccgatttcggcctattggttaaaaaatgagctgatttaacaaa
aatttaacgcgaattttaacaaaatattaacgcttacaatttaggtggcacttttcggggaaatgtgcgc
ggaacccctatttgtttatttttctaaatacattcaaatatgtatccgctcatgagacaataaccctgat
aaatgcttcaataatattgaaaaaggaagagtatgagtattcaacatttccgtgtcgcccttattccctt
ttttgcggcattttgccttcctgtttttgctcacccagaaacgctggtgaaagtaaaagatgctgaagat
cagttgggtgcacgagtgggttacatcgaactggatctcaacagcggtaagatccttgagagttttcgcc
ccgaagaacgttttccaatgatgagcacttttaaagttctgctatgtggcgcggtattatcccgtattga
cgccgggcaagagcaactcggtcgccgcatacactattctcagaatgacttggttgagtactcaccagtc
acagaaaagcatcttacggatggcatgacagtaagagaattatgcagtgctgccataaccatgagtgata
acactgcggccaacttacttctgacaacgatcggaggaccgaaggagctaaccgcttttttgcacaacat
gggggatcatgtaactcgccttgatcgttgggaaccggagctgaatgaagccataccaaacgacgagcgt
gacaccacgatgcctgtagcaatggcaacaacgttgcgcaaactattaactggcgaactacttactctag
cttcccggcaacaattaatagactggatggaggcggataaagttgcaggaccacttctgcgctcggccct
tccggctggctggtttattgctgataaatctggagccggtgagcgtgggtctcgcggtatcattgcagca
ctggggccagatggtaagccctcccgtatcgtagttatctacacgacggggagtcaggcaactatggatg
aacgaaatagacagatcgctgagataggtgcctcactgattaagcattggtaactgtcagaccaagttta
ctcatatatactttagattgatttaaaacttcatttttaatttaaaaggatctaggtgaagatccttttt
gataatctcatgaccaaaatcccttaacgtgagttttcgttccactgagcgtcagaccccgtagaaaaga
tcaaaggatcttcttgagatcctttttttctgcgcgtaatctgctgcttgcaaacaaaaaaaccaccgct
accagcggtggtttgtttgccggatcaagagctaccaactctttttccgaaggtaactggcttcagcaga
gcgcagataccaaatactgttcttctagtgtagccgtagttaggccaccacttcaagaactctgtagcac
cgcctacatacctcgctctgctaatcctgttaccagtggctgctgccagtggcgataagtcgtgtcttac
cgggttggactcaagacgatagttaccggataaggcgcagcggtcgggctgaacggggggttcgtgcaca
cagcccagcttggagcgaacgacctacaccgaactgagatacctacagcgtgagctatgagaaagcgcca
cgcttcccgaagggagaaaggcggacaggtatccggtaagcggcagggtcggaacaggagagcgcacgag
ggagcttccagggggaaacgcctggtatctttatagtcctgtcgggtttcgccacctctcacttgagcgt
cgatttttgtgatgctcgtcaggggggcggagcctatggaaaaacgccagcaacgcggcctttttacggt
tcctggccttttgctggccttttgctcacatg
GLUT5 4P28z amino acid sequence
SEQ ID NO: 16
MEQQDQSMKEGRLTLVLALATLIAAFGSSFQYGYNVAAVNSPALLMQQFYNETYYGRTGEFMEDFPLTLL
WSVTVSMFPFGGFIGSLLVGPLVNKFGRKGALLFNNIFSIVPAILMGCSRVATSFELIIISRLLVGICAG
VSSNVVPMYLGELAPKNLRGALGVVPQLFITVGILVAQIFGLRNLLANVDGWPILLGLTGVPAALQLLLL
PFFPESPRYLLIQKKDEAAAKKALQTLRGWDSVDREVAEIRQEDEAEKAAGFISVLKLFRMRSLRWQLLS
IIVLMGGQQLSGVNAIYYYADQIYLSAGVPEEHVQYVTAGTGAVNVVMTFCAVFVVELLGRRLLLLLGFS
ICLIACCVLTAALALQDTVSWMPYISIVCVISYVIGHALGPSPIPALLITEIFLQSSRPSAFMVGGSVHW
LSNFTVGLIFPFIQEGLGPYSFIVFAVICLLTTIYIFLIVPETKAKTFIEINQIFTKMNKVSEVYPEKEE
LKELPPVTSEQGSGATNFSLLKQAGDVEENPGPMGWLCSGLLFPVSCLVLLQVASSGNMKVLQEPTCVSD
YMSISTCEWKMNGPTNCSTELRLLYQLVFLLSEAHTCIPENNGGAGCVCHLLMDDVVSADNYTLDLWAGQ
QLLWKGSFKPSEHVKPRAPGNLTVHTNVSDTLLLTWSNPYPPDNYLYNHLTYAVNIWSENDPADFRIYNV
TYLEPSLRIAASTLKSGISYRARVRAWAQCYNTTWSEWSPSTKWHNSYREPFEQHLIPWLGHLLVGLSGA
FGFIILVYLLINCRNTGPWLKKVLKCNTPDPSKFFSQLSSEHGGDVQKWLSSPFPSSSFSPGGLAPEISP
LEVLERDKVTQLLLQQDKVPEPASLSSNHSLTSCFTNQGYFFFHLPDALEIEACQVYFTYDPYSEEDPDE
GVAGAPTGSSPQPLQPLSGEDDAYCTFPSRDDLLLFSPSLLGGPSPPSTAPGGSGAGEERMPPSLQERVP
RDWDPQPLGPPTPGVPDLVDFQPPPELVLREAGEEVPDAGPREGVSFPWSRPPGQGEFRALNARLPLNTD
AYLSLQELQGQDPTHLVRRKRSGSGEGRGSLLTCGDVEENPGPMALPVTALLLPLALLLHAEVQLQQSGP
ELVKPGTSVRISCKTSGYTFTEYTIHWVKQSHGKSLEWIGNINPNNGGTTYNQKFEDKATLTVDKSSSTA
YMELRSLTSEDSAVYYCAAGWNFDYWGQGTTVTVSSGGGGSGGGGSGGGGSDIVMTQSHKFMSTSVGDRV
SIICKASQDVGTAVDWYQQKPGQSPKLLIYWASTRHTGVPDRFTGSGSGTDFTLTITNVQSEDLADYFCQ
QYNSYPLTFGAGTMLDLKRAAAIEVEQKLISEEDLLDNEKSNGTIIHVKGKHLCPSPLFPGPSKPFWVLV
VVGGVLACYSLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
ADA2TM P28z nucleotide sequence
SEQ ID NO: 17
ATGTTGGTGGATGGCCCATCTGAGCGGCCAGCCCTGTGCTTCTTGCTGTTGGCTGTGGCAATGTCTTTCT
TCGGCTCAGCTCTATCCATAGATGAAACACGGGCGCATCTGTTGTTGAAAGAAAAGATGATGCGGCTGGG
GGGGCGGCTGGTGCTGAACACCAAGGAGGAGCTGGCCAATGAGAGGCTCATGACGCTCAAAATCGCTGAG
ATGAAGGAGGCCATGAGGACCCTGATATTCCCACCCAGCATGCACTTTTTCCAGGCCAAGCATCTCATTG
AGAGAAGTCAAGTGTTTAATATTCTAAGGATGATGCCAAAAGGGGCTGCCTTGCACCTCCATGACATTGG
CATCGTGACTATGGACTGGCTGGTGAGGAATGTCACCTACAGGCCTCACTGCCACATCTGTTTCACCCCA
AGGGGGATCATGCAGTTCAGATTTGCTCACCCAACTCCCCGTCCATCAGAAAAATGTTCCAAGTGGATTC
TGCTGGAGGATTATCGGAAGCGGGTGCAGAACGTCACTGAGTTTGATGACAGCTTGCTGAGGAATTTCAC
TCTGGTGACCCAGCACCCGGAGGTGATTTACACAAACCAAAATGTTGTCTGGTCGAAATTTGAAACCATC
TTCTTCACCATCTCTGGTCTCATCCATTACGCACCAGTGTTCAGAGACTATGTCTTCCGGAGCATGCAGG
AGTTCTACGAGGACAACGTGCTCTACATGGAGATCAGAGCCAGGCTGCTGCCGGTGTATGAGCTCAGTGG
AGAGCACCATGACGAAGAGTGGTCAGTGAAGACTTACCAGGAAGTAGCTCAGAAGTTTGTGGAAACTCAC
CCTGAGTTTATTGGAATCAAAATCATTTATTCGGATCACAGATCCAAAGATGTGGCTGTCATCGCAGAAT
CCATCCGAATGGCCATGGGGCTCCGAATCAAGTTCCCCACGGTGGTGGCAGGGTTTGACCTGGTGGGGCA
TGAGGACACTGGCCACTCCTTGCATGACTACAAGGAAGCTCTGATGATCCCCGCCAAGGATGGCGTTAAG
CTGCCTTACTTCTTCCACGCCGGAGAAACAGACTGGCAGGGTACTTCCATAGACAGGAACATTCTGGATG
CTCTGATGCTGAACACTACCAGAATCGGCCATGGATTTGCTTTGAGCAAACACCCCGCAGTCAGGACTTA
CTCCTGGAAAAAGGACATCCCCATAGAAGTCTGTCCCATCTCTAACCAGGTGCTGAAACTGGTGTCTGAC
TTGAGGAACCACCCTGTAGCCACTCTGATGGCCACTGGGCACCCCATGGTGATCAGCTCTGATGACCCAG
CTATGTTTGGTGCCAAAGGCTTGTCCTATGATTTCTATGAGGTCTTCATGGGCATTGGGGGGATGAAGGC
TGACCTGAGGACCCTCAAACAGCTGGCCATGAACTCTATCAAGTACAGTACCCTGTTGGAGAGTGAGAAA
AATACTTTCATGGAAATCTGGAAGAAGAGATGGGATAAGTTCATAGCAGATGTGGCTACAAAGGGCGGCG
GAGGCTCCGGCGGAGGCGGAAGCGGCGGCGGAGGATCTGACTACAAGGACGACGATGACAAGaccacgac
gccagcgccgcgaccaccaacaccggcgcccaccatcgcgtcgcagcccctgtccctgcgcccagaggcg
tgccggccagcggcggggggcgcagtgcacacgagggggctggacttcgcctgtgatatctacatctggg
cgcccttggccgggacttgtggggtccttctcctgtcactggttatcaccctttactgcggatctggagc
aacaaacttctcactactcaaacaagcaggtgacgtggaggagaatcccgggcctatggCTCTCCCAGTG
ACTGCCCTACTGCTTCCCCTAGCGCTTCTCCTGCATGCAGAGGTGCAGCTGCAGcagtcaggacctgaac
tggtgaagcctgggacttcagtgaggatatcctgcaagacttctggatacacattcactgaatataccat
acactgggtgaagcagagccatggaaagagccttgagtggattggaaacatcaatcctaacaatggtggt
accacctacaatcagaagttcgaggacaaggccacattgactgtagacaagtcctccagtacagcctaca
tggagctccgcagcctaacatctgaggattctgcagtctattattgtgcagctggttggaactttgacta
ctggggccaagggaccacGGTCACCgtctcctcaggtggaggTggAtcaggTggaggtggAtctggTggA
ggTggatcTGACATTGTGATGACCCAGTCTCACAAATTCATGTCCACATCAGTAGGAGACAGGGTCAGCA
TCATCTGTAAGGCCAGTCAAGATGTGGGTACTGCTGTAGACTGGTATCAACAGAAACCAGGACAATCTCC
TAAACTACTGATTTATTGGGCATCCACTCGGCACACTGGAGTCCCTGATCGCTTCACAGGCAGTGGATCT
GGGACAGACTTCACTCTCACCATTACTAATGTTCAGTCTGAAGACTTGGCAGATTATTTCTGTCAGCAAT
ATAACAGCTATCCCCTCACGTTCGGTGCTGGGACCATGCTGGACCTGAAACGGgcGGCCGCCATCGAGGT
GGAGCAGAAGCTGATCAGCGAGGAGGACCTGCTGGACAACGAGAAGAGCAACGGCACCATCATCCACGTG
AAGGGCAAGCACCTGTGCCCCAGCCCCCTGTTCCCCGGCCCCAGCAAGCCCTTCTGGGTGCTGGTGGTGG
TGGGCGGCGTGCTGGCCTGCTACAGCCTGCTGGTGACCGTGGCCTTCATCATCTTCTGGGTGCGGAGCAA
GCGGAGCCGGCTGCTGCACAGCGACTACATGAACATGACCCCCCGGCGGCCTGGGCCCACCCGCAAGCAT
TACCAGCCCTATGCCCCACCACGCGACTTCGCAGCCTATCGCTCCAGAGTGAAGTTCAGCAGGAGCGCAG
ACGCCCCCGCGTACCAGCAGGGCCAGAACCAGCTCTATAACGAGCTCAATCTAGGACGAAGAGAGGAGTA
CGATGTTTTGGACAAGAGACGTGGCCGGGACCCTGAGATGGGGGGAAAGCCGAGAAGGAAGAACCCTCAG
GAAGGCCTGTACAATGAACTGCAGAAAGATAAGATGGCGGAGGCCTACAGTGAGATTGGGATGAAAGGCG
AGCGCCGGAGGGGCAAgGGGCACGATGGCCTTTACCAGGGTCTCAGTACAGCCACCAAGGACACCTACGA
CGCCCTTCACATGCAGGCCCTGCCCCCTCGC
ADA2TM P28z amino acid sequence
SEQ ID NO: 18
MLVDGPSERPALCFLLLAVAMSFFGSALSIDETRAHLLLKEKMMRLGGRLVLNTKEELANERLMTLKIAE
MKEAMRTLIFPPSMHFFQAKHLIERSQVFNILRMMPKGAALHLHDIGIVTMDWLVRNVTYRPHCHICFTP
RGIMQFRFAHPTPRPSEKCSKWILLEDYRKRVQNVTEFDDSLLRNFTLVTQHPEVIYTNQNVVWSKFETI
FFTISGLIHYAPVFRDYVFRSMQEFYEDNVLYMEIRARLLPVYELSGEHHDEEWSVKTYQEVAQKFVETH
PEFIGIKIIYSDHRSKDVAVIAESIRMAMGLRIKFPTVVAGFDLVGHEDTGHSLHDYKEALMIPAKDGVK
LPYFFHAGETDWQGTSIDRNILDALMLNTTRIGHGFALSKHPAVRTYSWKKDIPIEVCPISNQVLKLVSD
LRNHPVATLMATGHPMVISSDDPAMFGAKGLSYDFYEVFMGIGGMKADLRTLKQLAMNSIKYSTLLESEK
NTFMEIWKKRWDKFIADVATKGGGGSGGGGSGGGGSDYKDDDDKTTTPAPRPPTPAPTIASQPLSLRPEA
CRPAAGGAVHTRGLDFACDIYIWAPLAGTCGVLLLSLVITLYCGSGATNFSLLKQAGDVEENPGPMALPV
TALLLPLALLLHAEVQLQQSGPELVKPGTSVRISCKTSGYTFTEYTIHWVKQSHGKSLEWIGNINPNNGG
TTYNQKFEDKATLTVDKSSSTAYMELRSLTSEDSAVYYCAAGWNFDYWGQGTTVTVSSGGGGSGGGGSGG
GGSDIVMTQSHKFMSTSVGDRVSIICKASQDVGTAVDWYQQKPGQSPKLLIYWASTRHTGVPDRFTGSGS
GTDFTLTITNVQSEDLADYFCQQYNSYPLTFGAGTMLDLKRAAAIEVEQKLISEEDLLDNEKSNGTIIHV
KGKHLCPSPLFPGPSKPFWVLVVVGGVLACYSLLVTVAFIIFWVRSKRSRLLHSDYMNMTPRRPGPTRKH
YQPYAPPRDFAAYRSRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
HER-2 Forward Primer
SEQ ID NO: 19
GGAAGAGAACCCCGGACCCATGGGCCCCGGCGTGC
HER-2 Reverse Primer
SEQ ID NO: 20
cgacgactccggaacgaattctgaTTATCTGGGAGGCAGGGCCTGCATG
Gp-100 Forward Primer
SEQ ID NO: 21
GGAAGAGAACCCCGGACCCatggccctgccagtgaccg
Gp-100 Reverse Primer
SEQ ID NO: 22
Cgacgactccggaacgaattctgatcagcctctgctgtccttccgc
GLUT5 4H28z nucleotide sequence
SEQ ID NO: 23
ttctttcctgcgttatcccctgattctgtggataaccgtattaccgcctttgagtgagctgataccgctc
gccgcagccgaacgaccgagcgcagcgagtcagtgagcgaggaagcggaagagcgcccaatacgcaaacc
gcctctccccgcgcgttggccgattcattaatgcagctggcacgacaggtttcccgactggaaagcgggc
agtgagcgcaacgcaattaatgtgagttagctcactcattaggcaccccaggctttacactttatgcttc
cggctcgtatgttgtgtggaattgtgagcggataacaatttcacacaggaaacagctatgaccatgatta
cgccaagcgcgcaattaaccctcactaaagggaacaaaagctggagctgcaagcttaatgtagtcttatg
caatactcttgtagtcttgcaacatggtaacgatgagttagcaacatgccttacaaggagagaaaaagca
ccgtgcatgccgattggtggaagtaaggtggtacgatcgtgccttattaggaaggcaacagacgggtctg
acatggattggacgaaccactgaattgccgcattgcagagatattgtatttaagtgcctagctcgataca
taaacgggtctctctggttagaccagatctgagcctgggagctctctggctaactagggaacccactgct
taagcctcaataaagcttgccttgagtgcttcaagtagtgtgtgcccgtctgttgtgtgactctggtaac
tagagatccctcagacccttttagtcagtgtggaaaatctctagcagtggcgcccgaacagggacttgaa
agcgaaagggaaaccagaggagctctctcgacgcaggactcggcttgctgaagcgcgcacggcaagaggc
gaggggcggcgactggtgagtacgccaaaaattttgactagcggaggctagaaggagagagatgggtgcg
agagcgtcagtattaagcgggggagaattagatcgcgatgggaaaaaattcggttaaggccagggggaaa
gaaaaaatataaattaaaacatatagtatgggcaagcagggagctagaacgattcgcagttaatcctggc
ctgttagaaacatcagaaggctgtagacaaatactgggacagctacaaccatcccttcagacaggatcag
aagaacttagatcattatataatacagtagcaaccctctattgtgtgcatcaaaggatagagataaaaga
caccaaggaagctttagacaagatagaggaagagcaaaacaaaagtaagaccaccgcacagcaagcggcc
gctgatcttcagacctggaggaggagatatgagggacaattggagaagtgaattatataaatataaagta
gtaaaaattgaaccattaggagtagcacccaccaaggcaaagagaagagtggtgcagagagaaaaaagag
cagtgggaataggagctttgttccttgggttcttgggagcagcaggaagcactatgggcgcagcgtcaat
gacgctgacggtacaggccagacaattattgtctggtatagtgcagcagcagaacaatttgctgagggct
attgaggcgcaacagcatctgttgcaactcacagtctggggcatcaagcagctccaggcaagaatcctgg
ctgtggaaagatacctaaaggatcaacagctcctggggatttggggttgctctggaaaactcatttgcac
cactgctgtgccttggaatgctagttggagtaataaatctctggaacagatttggaatcacacgacctgg
atggagtgggacagagaaattaacaattacacaagcttaatacactccttaattgaagaatcgcaaaacc
agcaagaaaagaatgaacaagaattattggaattagataaatgggcaagtttgtggaattggtttaacat
aacaaattggctgtggtatataaaattattcataatgatagtaggaggcttggtaggtttaagaatagtt
tttgctgtactttctatagtgaatagagttaggcagggatattcaccattatcgtttcagacccacctcc
caaccccgaggggacccgacaggcccgaaggaatagaagaagaaggtggagagagagacagagacagatc
cattcgattagtgaacggatcggcactgcgtgcgccaattctgcagacaaatggcagtattcatccacaa
ttttaaaagaaaaggggggattggggggtacagtgcaggggaaagaatagtagacataatagcaacagac
atacaaactaaagaattacaaaaacaaattacaaaaattcaaaattttcgggtttattacagggacagca
gagatccagtttggttaattaacccgtgtcggctccagatctgctccggtgcccgtcagtgggcagagcg
cacatcgcccacagtccccgagaagttggggggaggggtcggcaattgaaccggtgcctagagaaggtgg
cgcggggtaaactgggaaagtgatgtcgtgtactggctccgcctttttcccgagggtgggggagaaccgt
atataagtgcagtagtcgccgtgaacgttctttttcgcaacgggtttgccgccagaacacaggtaagtgc
cgtgtgtggttcccgcgggcctggcctctttacgggttatggcccttgcgtgccttgaattacttccacg
cccctggctgcagtacgtgattcttgatcccgagcttcgggttggaagtgggtgggagagttcgaggcct
tgcgcttaaggagccccttcgcctcgtgcttgagttgaggcctggcttgggcgctggggccgccgcgtgc
gaatctggtggcaccttcgcgcctgtctcgctgctttcgataagtctctagccatttaaaatttttgatg
acctgctgcgacgctttttttctggcaagatagtcttgtaaatgcgggccaagatctgcacactggtatt
tcggtttttggggccgcgggcggcgacggggcccgtgcgtcccagcgcacatgttcggcgaggcggggcc
tgcgagcgcggccaccgagaatcggacgggggtagtctcaagctggccggcctgctctggtgcctggcct
cgcgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggggg
tgagtcacccacacaaaggaaaagggcctttccgtcctcagccgtcgcttcatgtgactccacggagtac
cgggcgccgtccaggcacctcgattagttctcgagcttttggagtacgtcgtctttaggttggggggagg
ggttttatgcgatggagtttccccacactgagtgggtggagactgaagttaggccagcttggcacttgat
gtaattctccttggaatttgccctttttgagtttggatcttggttcattctcaagcctcagacagtggtt
caaagtttttttcttccatttcaggtgtcgtgaaaactacccctaaaaGCTAGCgccaccATGGAGCAAC
AGGATCAGAGCATGAAGGAAGGGAGGCTGACGCTTGTGCTTGCCCTGGCAACCCTGATAGCTGCCTTTGG
GTCATCCTTCCAGTATGGGTACAACGTGGCTGCTGTCAACTCCCCAGCACTGCTCATGCAACAATTTTAC
AATGAGACTTACTATGGTAGGACCGGTGAATTCATGGAAGACTTCCCCTTGACGTTGCTGTGGTCTGTAA
CCGTGTCCATGTTTCCATTTGGAGGGTTTATCGGATCCCTCCTGGTCGGCCCCTTGGTGAATAAATTTGG
CAGAAAAGGGGCCTTGCTGTTCAACAACATATTTTCTATCGTGCCTGCGATCTTAATGGGATGCAGCAGA
GTCGCCACATCATTTGAGCTTATCATTATTTCCAGACTTTTGGTTGGAATATGTGCAGGTGTATCTTCCA
ACGTGGTCCCCATGTACTTAGGGGAGCTGGCCCCTAAAAACCTGCGGGGGGCTCTCGGGGTGGTGCCCCA
GCTCTTCATCACTGTTGGCATCCTTGTGGCCCAGATCTTTGGTCTTCGGAATCTCCTTGCAAACGTAGAT
GGCTGGCCGATCCTGCTGGGGCTGACCGGGGTCCCCGCGGCGCTGCAGCTCCTTCTGCTGCCCTTCTTCC
CCGAGAGCCCCAGGTACCTGCTGATTCAGAAGAAAGACGAAGCGGCCGCCAAGAAAGCCCTACAGACGCT
GCGCGGCTGGGACTCTGTGGACAGGGAGGTGGCCGAGATCCGGCAGGAGGATGAGGCAGAGAAGGCCGCG
GGCTTCATCTCCGTGCTGAAGCTGTTCCGGATGCGCTCGCTGCGCTGGCAGCTGCTGTCCATCATCGTCC
TCATGGGCGGCCAGCAGCTGTCGGGCGTCAACGCTATCTACTACTACGCGGACCAGATCTACCTGAGCGC
CGGCGTGCCGGAGGAGCACGTGCAGTACGTGACGGCCGGCACCGGGGCCGTGAACGTGGTCATGACCTTC
TGCGCCGTGTTCGTGGTGGAGCTCCTGGGTCGGAGGCTGCTGCTGCTGCTGGGCTTCTCCATCTGCCTCA
TAGCCTGCTGCGTGCTCACTGCAGCTCTGGCACTGCAGGACACAGTGTCCTGGATGCCATACATCAGCAT
CGTCTGTGTCATCTCCTACGTCATAGGACATGCCCTCGGGCCCAGTCCCATACCCGCGCTGCTCATCACT
GAGATCTTCCTGCAGTCCTCTCGGCCATCTGCCTTCATGGTGGGGGGCAGTGTGCACTGGCTCTCCAACT
TCACCGTGGGCTTGATCTTCCCGTTCATCCAGGAGGGCCTCGGCCCGTACAGCTTCATTGTCTTCGCCGT
GATCTGCCTCCTCACCACCATCTACATCTTCTTGATTGTCCCGGAGACCAAGGCCAAGACGTTCATAGAG
ATCAACCAGATTTTCACCAAGATGAATAAGGTGTCTGAAGTGTACCCGGAAAAGGAGGAACTGAAAGAGC
TTCCACCTGTCACTTCGGAACAGGGATCTGGAGCAACAAACTTCTCACTACTCAAACAAGCAGGTGACGT
GGAGGAGAATCCCGGGCCTATGGGCTGGCTGTGTTCCGGCCTGCTGTTTCCTGTGTCCTGTCTGGTGCTG
CTGCAGGTGGCCAGCTCCGGGAACATGAAAGTGCTGCAGGAGCCCACATGTGTGTCCGACTACATGTCCA
TCTCTACATGTGAGTGGAAGATGAACGGCCCCACAAACTGCTCTACCGAGCTGCGGCTGCTGTACCAGCT
GGTGTTTCTGCTGAGCGAGGCCCACACCTGTATCCCAGAAAATAATGGCGGGGCCGGGTGTGTGTGCCAC
CTGCTGATGGATGACGTGGTGTCTGCCGACAATTACACCCTGGACCTGTGGGCCGGACAGCAGCTGCTGT
GGAAGGGGTCCTTCAAACCCTCTGAGCACGTGAAGCCAAGGGCCCCCGGCAACCTGACAGTGCACACCAA
CGTGTCTGATACACTGCTGCTGACATGGAGCAATCCATACCCTCCTGACAACTACCTGTACAACCACCTG
ACCTACGCCGTGAATATCTGGAGCGAAAATGATCCTGCCGACTTTCGGATTTACAATGTGACCTATCTGG
AGCCCTCCCTGAGAATTGCCGCCTCTACCCTGAAATCTGGAATCTCCTACCGCGCCAGGGTGCGGGCCTG
GGCCCAGTGTTACAACACCACCTGGTCTGAGTGGAGCCCAAGCACCAAGTGGCACAATTCTTATCGGGAG
CCTTTTGAGCAGCACCTGATCCCCTGGCTGGGACACCTGCTGGTGGGGCTGTCTGGCGCCTTTGGCTTCA
TCATTCTGGTGTACCTGCTGATCAACTGTAGGAATACAGGCCCTTGGCTGAAGAAGGTGCTGAAGTGTAA
CACCCCCGACCCCTCTAAGTTCTTCAGCCAGCTGTCCTCTGAACACGGGGGAGATGTGCAGAAGTGGCTG
TCCAGCCCTTTCCCATCCAGCTCCTTTAGCCCCGGGGGCCTGGCCCCTGAGATCTCTCCACTGGAAGTGC
TGGAGCGGGACAAGGTGACCCAGCTGCTGCTGCAGCAGGACAAGGTGCCAGAACCCGCCTCCCTGAGCTC
CAACCACAGCCTGACATCTTGCTTTACAAATCAGGGATACTTCTTCTTCCACCTGCCCGATGCCCTGGAG
ATCGAGGCCTGCCAGGTGTACTTCACCTACGATCCCTACTCTGAGGAAGACCCAGATGAGGGCGTGGCCG
GGGCCCCAACCGGGTCCAGCCCACAGCCACTGCAGCCACTGTCCGGCGAAGATGACGCCTACTGCACATT
CCCTTCCAGGGATGACCTGCTGCTGTTCAGCCCATCTCTGCTGGGCGGACCCTCTCCTCCAAGCACAGCC
CCAGGGGGATCCGGCGCCGGGGAAGAGAGGATGCCCCCTAGCCTGCAGGAGCGCGTGCCCAGAGACTGGG
ACCCCCAGCCCCTGGGCCCTCCAACCCCTGGGGTGCCCGACCTGGTGGACTTCCAGCCTCCACCCGAGCT
GGTGCTGAGGGAGGCCGGCGAAGAGGTGCCCGACGCCGGCCCCCGGGAGGGCGTGTCCTTCCCTTGGTCC
AGACCTCCAGGACAGGGCGAGTTCCGCGCCCTGAACGCCAGGCTGCCTCTGAACACCGATGCCTACCTGT
CTCTGCAGGAACTGCAGGGCCAGGACCCAACCCACCTGGTGCGGAGAAAGCGCAGCGGCTCCGGCGAGGG
CCGGGGCAGCCTGCTGACCTGCGGCGACGTGGAAGAGAACCCCGGACCCATGGGCCCCGGCGTGCTGCTG
CTGCTGCTGGTGGCCACCGCCTGGCACGGCCAGGGCGGCGAGGTGCAGCTTCAGGAGTCAGGACCTGGCC
TTGTGAAACCCTCACAGTCACTCTCCCTCACCTGTTCTGTCACTGGTTACTCCATCACTACTGATTACTG
GGGCTGGATCCGGAAGTTCCCAGGAAATAAAATGGAGTGGATGGGATACATAAGCTACAGTGGTAGCACT
GGCTACAACCCATCTCTCAAAAGTCGAATCTCCATTACTAGAGACACATCGAAGAGTCAGTTCTTCCTGC
AGTTGAACTCTGTAACTACTGAGGACACAGCCACATATTACTGTGCAAGATACAGTAGCCTTGATTACTG
GGGCCGAGGAGTCATGGTCGCAGTCTCCTCAGGAGGAGGTTCTGGCGGTGGATCAGGAGGCGGTTCGGAT
GTTGTGATGACCCAGACACCACCGTCTTTGTCGGTTGCCATTGGACAATCAGTCTCCATCTCTTGCAAGT
CAAGTCAGAGCCTCGTATATAGTGATGGAAAGACATATTTGCATTGGTTATTACAGAGTCCTGGCAGGTC
TCCGAAGCGCCTAATCTATCAGGTGTCTAATCTGGGCTCTGGAGTCCCTGACAGGTTCAGTGGCACTGGA
TCACAGAAAGATTTTACACTTAAAATCAGCAGAGTGGAGGCTGAGGATTTGGGAGTTTACTACTGCGCGC
AAACTACACATTTTCCTCTCACGTTCGGTTCTGGGACCAAGCTGGAGATCAAACGGGCGGCCGCAATCGA
GGTGGAGCAGAAGCTGATCAGCGAGGAGGACCTGCTGGACAACGAGAAGAGCAACGGCACCATCATCCAC
GTGAAGGGCAAGCACCTGTGTCCCAGCCCTCTGTTCCCCGGCCCTAGCAAGCCCTTCTGGGTGCTGGTGG
TTGTGGGCGGAGTGCTGGCCTGCTACAGCCTGCTCGTGACCGTGGCCTTCATCATCTTCTGGGTGAGAAG
CAAGAGAAGCAGACTGCTGCACAGCGACTACATGAACATGACACCCAGAAGACCCGGACCCACCAGAAAG
CACTACCAGCCCTACGCTCCCCCTAGAGACTTCGCCGCCTACAGAAGCAGAGTGAAGTTCAGCAGAAGCG
CCGACGCTCCCGCCTACCAGCAAGGCCAGAACCAGCTGTACAACGAGCTGAACCTGGGCAGAAGAGAGGA
GTACGACGTGCTGGACAAGAGAAGAGGCAGAGACCCTGAGATGGGAGGCAAGCCCAGAAGAAAGAACCCC
CAGGAGGGCCTGTACAACGAGCTGCAGAAGGACAAGATGGCCGAGGCCTACAGCGAGATCGGCATGAAGG
GAGAGAGAAGAAGAGGCAAGGGCCACGACGGCCTGTACCAGGGCCTGAGCACCGCCACCAAGGACACCTA
CGACGCCCTGCACATGCAGGCCCTGCCTCCCAGATAAtcagaattcgttccggagtcgtcgactcgacaa
tcaacctctggattacaaaatttgtgaaagattgactggtattcttaactatgttgctccttttacgcta
tgtggatacgctgctttaatgcctttgtatcatgctattgcttcccgtatggctttcattttctcctcct
tgtataaatcctggttgctgtctctttatgaggagttgtggcccgttgtcaggcaacgtggcgtggtgtg
cactgtgtttgctgacgcaacccccactggttggggcattgccaccacctgtcagctcctttccgggact
ttcgctttccccctccctattgccacggcggaactcatcgccgcctgccttgcccgctgctggacagggg
ctcggctgttgggcactgacaattccgtggtgttgtcggggaagctgacgtcctttccatggctgctcgc
ctgtgttgccacctggattctgcgcgggacgtccttctgctacgtcccttcggccctcaatccagcggac
cttccttcccgcggcctgctgccggctctgcggcctcttccgcatcttcgccttcgccctcagacgagtc
ggatctccctttgggccgcctccccgcctggaattaattcgagctcggtacctttaagaccaatgactta
caaggcagctgtagatcttagccactttttaaaagaaaaggggggactggaagggctacgtaactcccaa
cgaagacaagatctgctttttgcttgtactgggtctctctggttagaccagatctgagcctgggagctct
ctggctaactagggaacccactgcttaagcctcaataaagcttgccttgagtgcttcaagtagtgtgtgc
ccgtctgttgtgtgactctggtaactagagatccctcagacccttttagtcagtgtggaaaatctctagc
agtagtagttcatgtcatcttattattcagtatttataacttgcaaagaaatgaatatcagagagtgaga
ggaacttgtttattgcagcttataatggttacaaataaagcaatagcatcacaaatttcacaaataaagc
atttttttcactgcattctagttgtggtttgtccaaactcatcaatgtatcttatcatgtctggctctag
ctatcccgcccctaactccgcccatcccgcccctaactccgcccagttccgcccattctccgccccatgg
ctgactaattttttttatttatgcagaggccgaggccgcctcggcctctgagctattccagaagtagtga
ggaggcttttttggaggcctagggacgtacccaattcgccctatagtgagtcgtattacgcgcgctcact
ggccgtcgttttacaacgtcgtgactgggaaaaccctggcgttacccaacttaatcgccttgcagcacat
ccccctttcgccagctggcgtaatagcgaagaggcccgcaccgatcgcccttcccaacagttgcgcagcc
tgaatggcgaatgggacgcgccctgtagcggcgcattaagcgcggcgggtgtggtggttacgcgcagcgt
gaccgctacacttgccagcgccctagcgcccgctcctttcgctttcttcccttcctttctcgccacgttc
gccggctttccccgtcaagctctaaatcgggggctccctttagggttccgatttagtgctttacggcacc
tcgaccccaaaaaacttgattagggtgatggttcacgtagtgggccatcgccctgatagacggtttttcg
ccctttgacgttggagtccacgttctttaatagtggactcttgttccaaactggaacaacactcaaccct
atctcggtctattcttttgatttataagggattttgccgatttcggcctattggttaaaaaatgagctga
tttaacaaaaatttaacgcgaattttaacaaaatattaacgcttacaatttaggtggcacttttcgggga
aatgtgcgcggaacccctatttgtttatttttctaaatacattcaaatatgtatccgctcatgagacaat
aaccctgataaatgcttcaataatattgaaaaaggaagagtatgagtattcaacatttccgtgtcgccct
tattcccttttttgcggcattttgccttcctgtttttgctcacccagaaacgctggtgaaagtaaaagat
gctgaagatcagttgggtgcacgagtgggttacatcgaactggatctcaacagcggtaagatccttgaga
gttttcgccccgaagaacgttttccaatgatgagcacttttaaagttctgctatgtggcgcggtattatc
ccgtattgacgccgggcaagagcaactcggtcgccgcatacactattctcagaatgacttggttgagtac
tcaccagtcacagaaaagcatcttacggatggcatgacagtaagagaattatgcagtgctgccataacca
tgagtgataacactgcggccaacttacttctgacaacgatcggaggaccgaaggagctaaccgctttttt
gcacaacatgggggatcatgtaactcgccttgatcgttgggaaccggagctgaatgaagccataccaaac
gacgagcgtgacaccacgatgcctgtagcaatggcaacaacgttgcgcaaactattaactggcgaactac
ttactctagcttcccggcaacaattaatagactggatggaggcggataaagttgcaggaccacttctgcg
ctcggcccttccggctggctggtttattgctgataaatctggagccggtgagcgtgggtctcgcggtatc
attgcagcactggggccagatggtaagccctcccgtatcgtagttatctacacgacggggagtcaggcaa
ctatggatgaacgaaatagacagatcgctgagataggtgcctcactgattaagcattggtaactgtcaga
ccaagtttactcatatatactttagattgatttaaaacttcatttttaatttaaaaggatctaggtgaag
atcctttttgataatctcatgaccaaaatcccttaacgtgagttttcgttccactgagcgtcagaccccg
tagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggc
ttcagcagagcgcagataccaaatactgttcttctagtgtagccgtagttaggccaccacttcaagaact
ctgtagcaccgcctacatacctcgctctgctaatcctgttaccagtggctgctgccagtggcgataagtc
gtgtcttaccgggttggactcaagacgatagttaccggataaggcgcagcggtcgggctgaacggggggt
tcgtgcacacagcccagcttggagcgaacgacctacaccgaactgagatacctacagcgtgagctatgag
aaagcgccacgcttcccgaagggagaaaggcggacaggtatccggtaagcggcagggtcggaacaggaga
gcgcacgagggagcttccagggggaaacgcctggtatctttatagtcctgtcgggtttcgccacctctca
cttgagcgtcgatttttgtgatgctcgtcaggggggcggagcctatggaaaaacgccagcaacgcggcct
ttttacggttcctggccttttgctggccttttgctcacatg
GLUT5-4GP100 nucleotide sequence
SEQ ID NO: 24
ttctttcctgcgttatcccctgattctgtggataaccgtattaccgcctttgagtgagctgataccgctc
gccgcagccgaacgaccgagcgcagcgagtcagtgagcgaggaagcggaagagcgcccaatacgcaaacc
gcctctccccgcgcgttggccgattcattaatgcagctggcacgacaggtttcccgactggaaagcgggc
agtgagcgcaacgcaattaatgtgagttagctcactcattaggcaccccaggctttacactttatgcttc
cggctcgtatgttgtgtggaattgtgagcggataacaatttcacacaggaaacagctatgaccatgatta
cgccaagcgcgcaattaaccctcactaaagggaacaaaagctggagctgcaagcttaatgtagtcttatg
caatactcttgtagtcttgcaacatggtaacgatgagttagcaacatgccttacaaggagagaaaaagca
ccgtgcatgccgattggtggaagtaaggtggtacgatcgtgccttattaggaaggcaacagacgggtctg
acatggattggacgaaccactgaattgccgcattgcagagatattgtatttaagtgcctagctcgataca
taaacgggtctctctggttagaccagatctgagcctgggagctctctggctaactagggaacccactgct
taagcctcaataaagcttgccttgagtgcttcaagtagtgtgtgcccgtctgttgtgtgactctggtaac
tagagatccctcagacccttttagtcagtgtggaaaatctctagcagtggcgcccgaacagggacttgaa
agcgaaagggaaaccagaggagctctctcgacgcaggactcggcttgctgaagcgcgcacggcaagaggc
gaggggcggcgactggtgagtacgccaaaaattttgactagcggaggctagaaggagagagatgggtgcg
agagcgtcagtattaagcgggggagaattagatcgcgatgggaaaaaattcggttaaggccagggggaaa
gaaaaaatataaattaaaacatatagtatgggcaagcagggagctagaacgattcgcagttaatcctggc
ctgttagaaacatcagaaggctgtagacaaatactgggacagctacaaccatcccttcagacaggatcag
aagaacttagatcattatataatacagtagcaaccctctattgtgtgcatcaaaggatagagataaaaga
caccaaggaagctttagacaagatagaggaagagcaaaacaaaagtaagaccaccgcacagcaagcggcc
gctgatcttcagacctggaggaggagatatgagggacaattggagaagtgaattatataaatataaagta
gtaaaaattgaaccattaggagtagcacccaccaaggcaaagagaagagtggtgcagagagaaaaaagag
cagtgggaataggagctttgttccttgggttcttgggagcagcaggaagcactatgggcgcagcgtcaat
gacgctgacggtacaggccagacaattattgtctggtatagtgcagcagcagaacaatttgctgagggct
attgaggcgcaacagcatctgttgcaactcacagtctggggcatcaagcagctccaggcaagaatcctgg
ctgtggaaagatacctaaaggatcaacagctcctggggatttggggttgctctggaaaactcatttgcac
cactgctgtgccttggaatgctagttggagtaataaatctctggaacagatttggaatcacacgacctgg
atggagtgggacagagaaattaacaattacacaagcttaatacactccttaattgaagaatcgcaaaacc
agcaagaaaagaatgaacaagaattattggaattagataaatgggcaagtttgtggaattggtttaacat
aacaaattggctgtggtatataaaattattcataatgatagtaggaggcttggtaggtttaagaatagtt
tttgctgtactttctatagtgaatagagttaggcagggatattcaccattatcgtttcagacccacctcc
caaccccgaggggacccgacaggcccgaaggaatagaagaagaaggtggagagagagacagagacagatc
cattcgattagtgaacggatcggcactgcgtgcgccaattctgcagacaaatggcagtattcatccacaa
ttttaaaagaaaaggggggattggggggtacagtgcaggggaaagaatagtagacataatagcaacagac
atacaaactaaagaattacaaaaacaaattacaaaaattcaaaattttcgggtttattacagggacagca
gagatccagtttggttaattaacccgtgtcggctccagatctgctccggtgcccgtcagtgggcagagcg
cacatcgcccacagtccccgagaagttggggggaggggtcggcaattgaaccggtgcctagagaaggtgg
cgcggggtaaactgggaaagtgatgtcgtgtactggctccgcctttttcccgagggtgggggagaaccgt
atataagtgcagtagtcgccgtgaacgttctttttcgcaacgggtttgccgccagaacacaggtaagtgc
cgtgtgtggttcccgcgggcctggcctctttacgggttatggcccttgcgtgccttgaattacttccacg
cccctggctgcagtacgtgattcttgatcccgagcttcgggttggaagtgggtgggagagttcgaggcct
tgcgcttaaggagccccttcgcctcgtgcttgagttgaggcctggcttgggcgctggggccgccgcgtgc
gaatctggtggcaccttcgcgcctgtctcgctgctttcgataagtctctagccatttaaaatttttgatg
acctgctgcgacgctttttttctggcaagatagtcttgtaaatgcgggccaagatctgcacactggtatt
tcggtttttggggccgcgggcggcgacggggcccgtgcgtcccagcgcacatgttcggcgaggcggggcc
tgcgagcgcggccaccgagaatcggacgggggtagtctcaagctggccggcctgctctggtgcctggcct
cgcgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggggg
tgagtcacccacacaaaggaaaagggcctttccgtcctcagccgtcgcttcatgtgactccacggagtac
cgggcgccgtccaggcacctcgattagttctcgagcttttggagtacgtcgtctttaggttggggggagg
ggttttatgcgatggagtttccccacactgagtgggtggagactgaagttaggccagcttggcacttgat
gtaattctccttggaatttgccctttttgagtttggatcttggttcattctcaagcctcagacagtggtt
caaagtttttttcttccatttcaggtgtcgtgaaaactacccctaaaaGCTAGCgccaccATGGAGCAAC
AGGATCAGAGCATGAAGGAAGGGAGGCTGACGCTTGTGCTTGCCCTGGCAACCCTGATAGCTGCCTTTGG
GTCATCCTTCCAGTATGGGTACAACGTGGCTGCTGTCAACTCCCCAGCACTGCTCATGCAACAATTTTAC
AATGAGACTTACTATGGTAGGACCGGTGAATTCATGGAAGACTTCCCCTTGACGTTGCTGTGGTCTGTAA
CCGTGTCCATGTTTCCATTTGGAGGGTTTATCGGATCCCTCCTGGTCGGCCCCTTGGTGAATAAATTTGG
CAGAAAAGGGGCCTTGCTGTTCAACAACATATTTTCTATCGTGCCTGCGATCTTAATGGGATGCAGCAGA
GTCGCCACATCATTTGAGCTTATCATTATTTCCAGACTTTTGGTTGGAATATGTGCAGGTGTATCTTCCA
ACGTGGTCCCCATGTACTTAGGGGAGCTGGCCCCTAAAAACCTGCGGGGGGCTCTCGGGGTGGTGCCCCA
GCTCTTCATCACTGTTGGCATCCTTGTGGCCCAGATCTTTGGTCTTCGGAATCTCCTTGCAAACGTAGAT
GGCTGGCCGATCCTGCTGGGGCTGACCGGGGTCCCCGCGGCGCTGCAGCTCCTTCTGCTGCCCTTCTTCC
CCGAGAGCCCCAGGTACCTGCTGATTCAGAAGAAAGACGAAGCGGCCGCCAAGAAAGCCCTACAGACGCT
GCGCGGCTGGGACTCTGTGGACAGGGAGGTGGCCGAGATCCGGCAGGAGGATGAGGCAGAGAAGGCCGCG
GGCTTCATCTCCGTGCTGAAGCTGTTCCGGATGCGCTCGCTGCGCTGGCAGCTGCTGTCCATCATCGTCC
TCATGGGCGGCCAGCAGCTGTCGGGCGTCAACGCTATCTACTACTACGCGGACCAGATCTACCTGAGCGC
CGGCGTGCCGGAGGAGCACGTGCAGTACGTGACGGCCGGCACCGGGGCCGTGAACGTGGTCATGACCTTC
TGCGCCGTGTTCGTGGTGGAGCTCCTGGGTCGGAGGCTGCTGCTGCTGCTGGGCTTCTCCATCTGCCTCA
TAGCCTGCTGCGTGCTCACTGCAGCTCTGGCACTGCAGGACACAGTGTCCTGGATGCCATACATCAGCAT
CGTCTGTGTCATCTCCTACGTCATAGGACATGCCCTCGGGCCCAGTCCCATACCCGCGCTGCTCATCACT
GAGATCTTCCTGCAGTCCTCTCGGCCATCTGCCTTCATGGTGGGGGGCAGTGTGCACTGGCTCTCCAACT
TCACCGTGGGCTTGATCTTCCCGTTCATCCAGGAGGGCCTCGGCCCGTACAGCTTCATTGTCTTCGCCGT
GATCTGCCTCCTCACCACCATCTACATCTTCTTGATTGTCCCGGAGACCAAGGCCAAGACGTTCATAGAG
ATCAACCAGATTTTCACCAAGATGAATAAGGTGTCTGAAGTGTACCCGGAAAAGGAGGAACTGAAAGAGC
TTCCACCTGTCACTTCGGAACAGGGATCTGGAGCAACAAACTTCTCACTACTCAAACAAGCAGGTGACGT
GGAGGAGAATCCCGGGCCTATGGGCTGGCTGTGTTCCGGCCTGCTGTTTCCTGTGTCCTGTCTGGTGCTG
CTGCAGGTGGCCAGCTCCGGGAACATGAAAGTGCTGCAGGAGCCCACATGTGTGTCCGACTACATGTCCA
TCTCTACATGTGAGTGGAAGATGAACGGCCCCACAAACTGCTCTACCGAGCTGCGGCTGCTGTACCAGCT
GGTGTTTCTGCTGAGCGAGGCCCACACCTGTATCCCAGAAAATAATGGCGGGGCCGGGTGTGTGTGCCAC
CTGCTGATGGATGACGTGGTGTCTGCCGACAATTACACCCTGGACCTGTGGGCCGGACAGCAGCTGCTGT
GGAAGGGGTCCTTCAAACCCTCTGAGCACGTGAAGCCAAGGGCCCCCGGCAACCTGACAGTGCACACCAA
CGTGTCTGATACACTGCTGCTGACATGGAGCAATCCATACCCTCCTGACAACTACCTGTACAACCACCTG
ACCTACGCCGTGAATATCTGGAGCGAAAATGATCCTGCCGACTTTCGGATTTACAATGTGACCTATCTGG
AGCCCTCCCTGAGAATTGCCGCCTCTACCCTGAAATCTGGAATCTCCTACCGCGCCAGGGTGCGGGCCTG
GGCCCAGTGTTACAACACCACCTGGTCTGAGTGGAGCCCAAGCACCAAGTGGCACAATTCTTATCGGGAG
CCTTTTGAGCAGCACCTGATCCCCTGGCTGGGACACCTGCTGGTGGGGCTGTCTGGCGCCTTTGGCTTCA
TCATTCTGGTGTACCTGCTGATCAACTGTAGGAATACAGGCCCTTGGCTGAAGAAGGTGCTGAAGTGTAA
CACCCCCGACCCCTCTAAGTTCTTCAGCCAGCTGTCCTCTGAACACGGGGGAGATGTGCAGAAGTGGCTG
TCCAGCCCTTTCCCATCCAGCTCCTTTAGCCCCGGGGGCCTGGCCCCTGAGATCTCTCCACTGGAAGTGC
TGGAGCGGGACAAGGTGACCCAGCTGCTGCTGCAGCAGGACAAGGTGCCAGAACCCGCCTCCCTGAGCTC
CAACCACAGCCTGACATCTTGCTTTACAAATCAGGGATACTTCTTCTTCCACCTGCCCGATGCCCTGGAG
ATCGAGGCCTGCCAGGTGTACTTCACCTACGATCCCTACTCTGAGGAAGACCCAGATGAGGGCGTGGCCG
GGGCCCCAACCGGGTCCAGCCCACAGCCACTGCAGCCACTGTCCGGCGAAGATGACGCCTACTGCACATT
CCCTTCCAGGGATGACCTGCTGCTGTTCAGCCCATCTCTGCTGGGCGGACCCTCTCCTCCAAGCACAGCC
CCAGGGGGATCCGGCGCCGGGGAAGAGAGGATGCCCCCTAGCCTGCAGGAGCGCGTGCCCAGAGACTGGG
ACCCCCAGCCCCTGGGCCCTCCAACCCCTGGGGTGCCCGACCTGGTGGACTTCCAGCCTCCACCCGAGCT
GGTGCTGAGGGAGGCCGGCGAAGAGGTGCCCGACGCCGGCCCCCGGGAGGGCGTGTCCTTCCCTTGGTCC
AGACCTCCAGGACAGGGCGAGTTCCGCGCCCTGAACGCCAGGCTGCCTCTGAACACCGATGCCTACCTGT
CTCTGCAGGAACTGCAGGGCCAGGACCCAACCCACCTGGTGCGGAGAAAGCGCAGCGGCTCCGGCGAGGG
CCGGGGCAGCCTGCTGACCTGCGGCGACGTGGAAGAGAACCCCGGACCCatggccctgccagtgaccgcc
ctgctgctgccactggccctgctgctgcacgcagcacggccaaagaatgaggtggagcagagcccacaga
acctgaccgcacaggagggagagttcatcacaatcaattgctcttacagcgtgggaatctccgccctgca
ctggctgcagcagcacccaggaggaggcatcgtgtctctgtttatgctgagctccggcaagaagaagcac
ggcaggctgatcgccaccatcaacatccaggagaagcactctagcctgcacatcacagcctcccaccctc
gcgattctgccgtgtatatctgtgcagcaagcctgatccagggagcacagaagctggtgttcggacaggg
caccaggctgacaatcaatcccccttacatccagaaccctgacccagccgtgtatcagctgcgcgactct
aagtcctctgataagagcgtgtgcctgttcaccgactttgattctcagacaaacgtgagccagtctaagg
acagcgacgtgtacatcaccgacaagtgcgtgctggatatgcggtccatggactttaagagcaattccgc
cgtggcctggagcaacaagtccgatttcgcctgcgccaacgcctttaacaattctatcatccctgaggac
acattctttcccagctccgacgtgccttgtgatgtgaagctggtggagaagtccttcgagaccgacacaa
atctgaactttcagaatctgctggtcatcgtgctgagaatcctgctgctgaaggtggtgggcttcaacct
gctgatgaccctgaggctgtggtctagcggctccggagagggaagaggctctctgctgacctgcggcgat
gtggaggagaatccaggacctatggcactgcctgtgacagccctgctgctgcctctggccctgctgctgc
atgcagcaaggcctgacgcaggcgtgatccagagcccaaggcacgaggtgaccgagatgggacaggaggt
gacactgaggtgtaagccaatctctggccacaatagcctgttctggtacaggcagaccatgatgcgcggc
ctggagctgctgatctacttcaacaataacgtgcccatcgacgatagcggcatgcctgaggatcggttct
ccgccaagatgcccaacgcctcttttagcacactgaagatccagcctagcgagccaagagactccgccgt
gtacttctgcgcctcctctcccggcggcaatgagcagttctttggccctggcaccaggctgacagtgctg
gatctgaagaacgtgttcccacccgaggtggccgtgtttgagccatccaaggccgagatcgcccacaccc
agaaggccaccctggtgtgcctggcaaccggcttctatcccgaccacgtggagctgtcctggtgggtgaa
tggcaaggaggtgcactctggcgtgtgcacagatccacagcccctgaaggagcagcctgccctgaacgac
agccgctactgtctgagctcccggctgagagtgtccgccaccttttggcagaatccaaggaaccacttcc
gctgccaggtgcagttttatggcctgagcgagaacgatgagtggacccaggacagggcaaagccagtgac
acagatcgtgtccgccgaggcatggggaagagcagattgtggcatcacctccgcctcttatcaccagggc
gtgctgagcgccaccatcctgtacgagatcctgctgggcaaggccacactgtatgccgtgctggtgtctg
ccctggtgctgatggccatggtgaagcggaaggacagcagaggctgatcagaattcgttccggagtcgtc
gactcgacaatcaacctctggattacaaaatttgtgaaagattgactggtattcttaactatgttgctcc
ttttacgctatgtggatacgctgctttaatgcctttgtatcatgctattgcttcccgtatggctttcatt
ttctcctccttgtataaatcctggttgctgtctctttatgaggagttgtggcccgttgtcaggcaacgtg
gcgtggtgtgcactgtgtttgctgacgcaacccccactggttggggcattgccaccacctgtcagctcct
ttccgggactttcgctttccccctccctattgccacggcggaactcatcgccgcctgccttgcccgctgc
tggacaggggctcggctgttgggcactgacaattccgtggtgttgtcggggaagctgacgtcctttccat
ggctgctcgcctgtgttgccacctggattctgcgcgggacgtccttctgctacgtcccttcggccctcaa
tccagcggaccttccttcccgcggcctgctgccggctctgcggcctcttccgcatcttcgccttcgccct
cagacgagtcggatctccctttgggccgcctccccgcctggaattaattcgagctcggtacctttaagac
caatgacttacaaggcagctgtagatcttagccactttttaaaagaaaaggggggactggaagggctacg
taactcccaacgaagacaagatctgctttttgcttgtactgggtctctctggttagaccagatctgagcc
tgggagctctctggctaactagggaacccactgcttaagcctcaataaagcttgccttgagtgcttcaag
tagtgtgtgcccgtctgttgtgtgactctggtaactagagatccctcagacccttttagtcagtgtggaa
aatctctagcagtagtagttcatgtcatcttattattcagtatttataacttgcaaagaaatgaatatca
gagagtgagaggaacttgtttattgcagcttataatggttacaaataaagcaatagcatcacaaatttca
caaataaagcatttttttcactgcattctagttgtggtttgtccaaactcatcaatgtatcttatcatgt
ctggctctagctatcccgcccctaactccgcccatcccgcccctaactccgcccagttccgcccattctc
cgccccatggctgactaattttttttatttatgcagaggccgaggccgcctcggcctctgagctattcca
gaagtagtgaggaggcttttttggaggcctagggacgtacccaattcgccctatagtgagtcgtattacg
cgcgctcactggccgtcgttttacaacgtcgtgactgggaaaaccctggcgttacccaacttaatcgcct
tgcagcacatccccctttcgccagctggcgtaatagcgaagaggcccgcaccgatcgcccttcccaacag
ttgcgcagcctgaatggcgaatgggacgcgccctgtagcggcgcattaagcgcggcgggtgtggtggtta
cgcgcagcgtgaccgctacacttgccagcgccctagcgcccgctcctttcgctttcttcccttcctttct
cgccacgttcgccggctttccccgtcaagctctaaatcgggggctccctttagggttccgatttagtgct
ttacggcacctcgaccccaaaaaacttgattagggtgatggttcacgtagtgggccatcgccctgataga
cggtttttcgccctttgacgttggagtccacgttctttaatagtggactcttgttccaaactggaacaac
actcaaccctatctcggtctattcttttgatttataagggattttgccgatttcggcctattggttaaaa
aatgagctgatttaacaaaaatttaacgcgaattttaacaaaatattaacgcttacaatttaggtggcac
ttttcggggaaatgtgcgcggaacccctatttgtttatttttctaaatacattcaaatatgtatccgctc
atgagacaataaccctgataaatgcttcaataatattgaaaaaggaagagtatgagtattcaacatttcc
gtgtcgcccttattcccttttttgcggcattttgccttcctgtttttgctcacccagaaacgctggtgaa
agtaaaagatgctgaagatcagttgggtgcacgagtgggttacatcgaactggatctcaacagcggtaag
atccttgagagttttcgccccgaagaacgttttccaatgatgagcacttttaaagttctgctatgtggcg
cggtattatcccgtattgacgccgggcaagagcaactcggtcgccgcatacactattctcagaatgactt
ggttgagtactcaccagtcacagaaaagcatcttacggatggcatgacagtaagagaattatgcagtgct
gccataaccatgagtgataacactgcggccaacttacttctgacaacgatcggaggaccgaaggagctaa
ccgcttttttgcacaacatgggggatcatgtaactcgccttgatcgttgggaaccggagctgaatgaagc
cataccaaacgacgagcgtgacaccacgatgcctgtagcaatggcaacaacgttgcgcaaactattaact
ggcgaactacttactctagcttcccggcaacaattaatagactggatggaggcggataaagttgcaggac
cacttctgcgctcggcccttccggctggctggtttattgctgataaatctggagccggtgagcgtgggtc
tcgcggtatcattgcagcactggggccagatggtaagccctcccgtatcgtagttatctacacgacgggg
agtcaggcaactatggatgaacgaaatagacagatcgctgagataggtgcctcactgattaagcattggt
aactgtcagaccaagtttactcatatatactttagattgatttaaaacttcatttttaatttaaaaggat
ctaggtgaagatcctttttgataatctcatgaccaaaatcccttaacgtgagttttcgttccactgagcg
tcagaccccgtagaaaagatcaaaggatcttcttgagatcctttttttctgcgcgtaatctgctgcttgc
aaacaaaaaaaccaccgctaccagcggtggtttgtttgccggatcaagagctaccaactctttttccgaa
ggtaactggcttcagcagagcgcagataccaaatactgttcttctagtgtagccgtagttaggccaccac
ttcaagaactctgtagcaccgcctacatacctcgctctgctaatcctgttaccagtggctgctgccagtg
gcgataagtcgtgtcttaccgggttggactcaagacgatagttaccggataaggcgcagcggtcgggctg
aacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcg
gaacaggagagcgcacgagggagcttccagggggaaacgcctggtatctttatagtcctgtcgggtttcg
ccacctctcacttgagcgtcgatttttgtgatgctcgtcaggggggcggagcctatggaaaaacgccagc
aacgcggcctttttacggttcctggccttttgctggccttttgctcacatg

1. An immune cell which is genetically engineered to express an exogenous alternative carbon source (ACS) metabolism gene, wherein the ACS is not glucose; and wherein the ability of the immune cell to metabolise the ACS is increased due to expression of the exogenous ACS metabolism gene.

2. The immune cell of claim 1, wherein the exogenous ACS metabolism gene does not encode a trehalose transporter.

3. The immune cell of claim 1 or claim 2, wherein the ACS is a monosaccharide, nucleoside or disaccharide.

4. The immune cell of claim 3, wherein the monosaccharide is fructose.

5. The immune cell of claim 3 or claim 4, wherein the nucleoside is selected from guanosine and adenosine.

6. The immune cell of any one of claims 3 to 5, wherein the disaccharide is selected from sucrose, lactose, maltose, isomaltose and trehalose.

7. The immune cell of any one of claims 3 to 6, wherein the disaccharide is trehalose.

8. The immune cell of any one of the preceding claims, wherein the ACS is trehalose, fructose or adenosine.

9. The immune cell of any one of the preceding claims, wherein the exogenous ACS metabolism gene encodes a transporter or enzyme.

10. The immune cell of claim 9, wherein the exogenous ACS metabolism gene encodes an enzyme selected from ADA, ADA2, LCT, MGAM, SI, Ketohexokinase (KHK), Aldolase B (ALDOB) and trehalase.

11. The immune cell of claim 9, wherein the exogenous ACS metabolism gene encodes GLUT5.

12. The immune cell of any one of the preceding claims, wherein the immune cell is selected from a B-cell, T-cell and Natural Killer (NK) cell.

13. The immune cell of claim 12, wherein the immune cell is a T-cell.

14. The immune cell of any one of the preceding claims, wherein the immune cell is a primary immune cell.

15. The immune cell of any one of the preceding claims, wherein the immune cell further expresses an exogenous TCR and/or a chimeric antigen receptor (CAR).

16. The immune cell of any one of the preceding claims, wherein the ACS metabolism gene is undetectable in cancer cells and/or cancer associated cells.

17. An isolated polynucleotide comprising an alternative carbon source (ACS) metabolism gene as defined in any one of claims 1 to 16 and a polynucleotide encoding a chimeric antigen receptor (CAR) and/or TCR.

18. The isolated polynucleotide of claim 17, wherein the ACS metabolism gene encodes an enzyme, wherein optionally the enzyme is selected from ADA, ADA2, LCT, MGAM, SI, Ketohexokinase (KHK), Aldolase B (ALDOB) and trehalase.

19. The isolated polynucleotide of claim 17 or claim 18, wherein the isolated polynucleotide comprises from 5′ to 3′ the ACS metabolism gene and the polynucleotide encoding the CAR and/or TCR.

20. The isolated polynucleotide of any one of claims 17 to 19, wherein the isolated polynucleotide comprises a polynucleotide encoding a ribosomal skipping sequence between the ACS metabolism gene and the polynucleotide encoding the CAR and/or TCR.

21. A vector comprising the isolated polynucleotide as defined in any one of claims 17 to 20.

22. The vector according to claim 21, wherein the vector is a retroviral or lentiviral vector, optionally an SFG retroviral vector.

23. A method of genetically engineering an immune cell, the method comprising introducing an exogenous ACS metabolism gene into the immune cell, wherein the ACS is not glucose.

24. The method of claim 23, wherein the exogenous ACS metabolism gene is introduced into the immune cell as the isolated polynucleotide of any one of claims 17 to 20 or the vector of claim 21 or 22.

25. A pharmaceutical composition comprising the immune cell of any one of claims 1 to 16, the isolated polynucleotide of any one of claims 17 to 20 and/or the vector of claim 21 or claim 22 and a pharmaceutically or physiologically acceptable diluent and/or carrier.

26. The immune cell of any one of claims 1 to 16, the isolated polynucleotide of any one of claims 17 to 20, the vector of claim 21 or claim 22 or the pharmaceutical composition of claim 25 for use in (i) therapy or (ii) the treatment of cancer.

27. A method of treating a disease in a subject, wherein the method comprises administering to the subject the immune cell of any one of claims 1 to 16, the isolated polynucleotide of any one of claims 17 to 20, the vector of claim 21 or claim 22 or the pharmaceutical composition of claim 25.

28. The method of claim 27, wherein the disease is cancer.
